(19) United States
(12) Patent Application Publication
(10) Pub. No.: US 2020/0030430 A1
(43) Pub. Date: Jan. 30, 2020

(54) IMMUNOGENIC COMPOSITIONS

(71) Applicant: GLAXOSMITHKLINE BIOLOGICALS S.A., Rixensart (BE)

(72) Inventors: Edmondo CAMPISI, Siena (IT); Immaculada MARGARIT Y ROS, Siena (IT); Roberto ROSINI, Siena (IT)

(73) Assignee: GLAXOSMITHKLINE BIOLOGICALS SA, Rixensart (BE)

(21) Appl. No.: 15/580,510

(22) PCT Filed: Jun. 30, 2016
(86) PCT No.: PCT/EP2016/065352

§ 371 Date: Dec. 7, 2017

(30) Foreign Application Priority Data
Jul. 1, 2015 (EP) 15020110.1
Nov. 5, 2015 (EP) 15193288.6

(51) Int. Cl. A61K 39/09; C12P 19/04

(52) U.S. Cl. CPC A61K 39/09; A61K 2039/55505; C12P 19/04

(57) ABSTRACT

Chimeric capsular polysaccharides, conjugates comprising such chimeric capsular polysaccharides, and pharmaceutical compositions thereof. Further aspects include methods for immunising a subject against infection by administration of such polysaccharides, conjugates, or compositions.

### TECHNICAL FIELD

This invention relates to chimeric capsular saccharides from Streptococcus agalactiae including conjugates comprising said chimeric capsular polysaccharides and carrier proteins.

### BACKGROUND TO THE INVENTION

Streptococcus agalactiae (also known as ‘Group B Streptococcus’ or ‘GBS’) is a β-hemolytic, encapsulated Gram-positive microorganism that colonizes the anogenital tract of 25-30% of healthy women. It is a major cause of neonatal sepsis and meningitis, particularly in infants born to mothers carrying the bacteria. The pathogen can also infect adults with underlying disease, particularly the elderly, and cause bovine mastitis. Streptococcus pneumoniae (also known as ‘S. pneumo’ or ‘pneumococcus’) is an alpha-hemolytic, encapsulated Gram-positive, microorganism that resides asymptomatically in the nasopharynx of healthy carriers. In susceptible individuals, such as the elderly, children and immunocompromised individuals, the bacterium may become pathogenic and cause disease such as pneumonia, meningitis or septicaemia.

The GBS capsule is a major virulence factor enabling the bacterium to evade human innate immune defences. It consists of high molecular weight polymers constituted by multiple identical repeating units (RUs) of four to seven monosaccharides. GBS can be classified into ten serotypes (Ia, Ib, II, III, IV, V, VI, VII, VIII, and IX) differing in the chemical composition and the pattern of glycosidic linkages of their capsular polysaccharide repeating units. Similarly, the capsule of S. pneumoniae is a major virulence factor consisting of high molecular weight polymers constituted by multiple identical repeating units. However, in contrast to GBS, more than 90 different serotypes of S. pneuminae have been identified to date.

The capsular saccharides of GBS and S. pneumoniae are being investigated for use in vaccines. However, saccharides are T-independent antigens and are generally poorly immunogenic. Therefore, conjugation to a carrier can convert T-independent antigens into T-dependent antigens, thereby enhancing memory responses and allowing protective immunity to develop. The most effective saccharide vaccines are therefore based on glycoconjugates. Much of the work on GBS capsular polysaccharide vaccines has been performed by Dennis Kasper and colleagues, and is described in documents such as references 1 to 9. Conjugate vaccines for each of GBS serotypes Ia, Ib, II, III, and V have been shown to be safe and immunogenic in humans [10]. Several vaccines for use in protecting S. pneumoniae infection are known and generally consist of purified polysaccharides selected from twenty three main serotypes (1, 2, 3, 4, 5, 6b, 7F, 8,9N, 9V, 10A, 11A, 12F, 14, 15B, 17F, 18C, 19F, 19A, 20, 22F, 23F, and 33F) and include, for example, Prevnar Synflorix® and Prevnar 13®.

However, whilst the capsular polysaccharides can induce a protective humoral response, the protection is highly specific for the specific serogroup (i.e. Ia, Ib, III, etc.) and does not confer cross-protection to other serogroups. Therefore, there remains a need for further and improved conjugate vaccines against GBS.

### BRIEF DESCRIPTION OF THE INVENTION

In a first aspect the invention provides chimeric capsular polysaccharides comprising at least one capsular polysaccharide repeating unit of a first serotype and at least one capsular polysaccharide repeating unit of a second, different serotype wherein the repeating units are joined by a glycosidic bond. The chimeric capsular polysaccharides are bacterial capsular polysaccharides. Particularly the chimeric capsular polysaccharide is a high molecular weight polymer yet more particularly having a molecular weight (MW) of >30 kDa, for example, a MW up to about or greater than 50 kDa, about or greater than 100 kDa, about or greater than 140 kDa, about or greater than 200 kDa, about or greater than 230 kDa, about or greater than 260 kDa or any range there between, for example, having a MW in the range of 50-200 kDa, 80-150 kDa, 150-300 kDa, 175-275 kDa or 175-250 kDa.

In some embodiments, the invention provides chimeric capsular polysaccharides comprising at least one capsular polysaccharide repeating unit of a first GBS serotype and at least one capsular polysaccharide repeating unit of a second, different GBS serotype wherein the repeating units are joined by a glycosidic bond. Particularly wherein the repeating units are present in a balanced epitope ratio, yet more particularly wherein the repeating units are present at a ratio of 1:1.

In particular embodiments, the invention provides chimeric capsular polysaccharide comprising at least one repeating unit of a first GBS capsular polysaccharide serotype, at least one repeating unit of a second GBS capsular polysaccharide serotype and at least one repeating unit of a third GBS capsular polysaccharide serotype, wherein the first, second and third repeating units are from different GBS capsular polysaccharide serotypes and wherein the repeating units are joined by glycosidic bonds.

Wildtype GBS capsular polysaccharides are homopolymers formed from identical repeating units joined by glycosidic bonds. In contrast, chimeric capsular polysaccharides of the present invention are heteropolymers formed from repeating units of at least two different GBS serotypes. More specifically, and since the chimeric polysaccharides are generated in vivo by individual bacterial cells, capsular polysaccharides of the invention are heteropolymers formed from repeating units having the structure of repeating units from at least two different GBS serotypes.

Particular chimeric polysaccharides comprise repeating units having the structure of repeating units from GBS capsular serotypes Ia+III, Ia+Ib+III, Ib+III, VII+IX, V+VII+IX, IV+V, V+VII and IV+V+VII.

In some embodiments, the invention provides chimeric capsular polysaccharides comprising repeating units joined by at least two, at least three, at least four or at least five different types of glycosidic bond. Particularly the glycosidic bonds are selected from the group consisting of β-d-Glcp-(1→4)-β-d-Galp, β-d-Glcp-(1→6)-β-d-GlcpNAc, β-d-Glcp-(1→6)-β-d-Glcp, β-d-Glcp-(1→2)-β-d-Galp and β-d-Glcp-(1→4)-α-d-Glcp.

In some embodiments, the invention provides chimeric capsular polysaccharides comprising at least one capsular polysaccharide repeating unit of a first Streptococcus pneumoniae serotype and at least one capsular polysaccharide repeating unit of a second, different Streptococcus pneumoniae serotype wherein the repeating units are joined by a glycosidic bond.

In a second aspect, the invention provides a conjugate comprising (i) a chimeric capsular polysaccharide of the first aspect and (ii) a carrier protein. Preferably the carrier protein is covalently bound to the capsular polysaccharide. In certain embodiments the carrier protein is covalently bound to the capsular polysaccharide via a linker, for example, adipic acid dihydrazide. Preferably the conjugate is an immunogenic conjugate capable of inducing an immune response against at least two different GBS serotypes, at least three different serotypes or more. Preferably the immune response is a protective immune response, for example, a cross-protective immune response.

In certain embodiments the carrier protein is selected from the group consisting of tetanus toxoid, diphtheria toxoid, CRM197, GBS80, GBS59 and GBS59 (6xD3)-1523.

In a third aspect the invention provides pharmaceutical compositions comprising the chimeric polysaccharide according to the first aspect and/or the conjugate according to the second aspect. Particularly, the chimeric polysaccharide and/or conjugate is in an amount effective to prevent systemic infections in an animal wherein said systemic infections are caused by group B streptococcus. Particularly, pharmaceutical compositions of the invention comprise a pharmaceutically acceptable diluent, carrier, or excipient. Yet more particularly, pharmaceutical compositions of the invention are vaccine compositions capable of eliciting an immune response against group B streptococcus.

In a fourth aspect, the invention provides a method of immunising a patient against infection by group B streptococcus comprising the step of administering to the patient a conjugate of the invention.

### BRIEF DESCRIPTION OF FIGURES

FIG. 1: provides a generalised structure of the cps operon, the CPS assembly genes are located in a long polycistronic operon that is largely conserved in many other encapsulated bacteria such as Streptococcus pneumoniae.

FIG. 2: provides a comparison of the generalised structures of the cps operon of a type III Streptococcus agalactiae serotype and a type 14 Streptococcus pneumoniae serotype.

FIG. 3: shows the structure of the repeating units of GBS capsular polysaccharides Ia, Ib, III.

FIG. 4: shows the structure of the repeating units of GBS capsular polysaccharides IV, V and VI.

FIG. 5: shows the structure of the repeating units of GBS capsular polysaccharides VII and VIII.

FIG. 6: shows the structure of the repeating units of GBS capsular polysaccharides IX and II.

FIG. 7: exemplifies a type Ia/III chimeric polysaccharide comprising type Ia repeating units and type III repeating units joined by β-d-Glcp-(1→4)-β-d-Galp and β-d-Glcp-(1→6)-β-d-GlcpNAc glycosidic bonds.

FIG. 8: provides an example of a type Ia/Ib/III chimeric polysaccharide that comprises type Ia, type III and type Ib repeating units joined by β-d-Glcp-(1→4)-β-d-Galp and β-d-Glcp-(1→6)-β-d-GlcpNAc glycosidic bonds.

FIG. 9: exemplifies a chimeric repeating unit obtained from serotype IX bacteria expressing cps5O. The additional side chain is shown in bold.

FIG. 10: provides a schematic of the CPS operon from different serotypes of Streptococcus agalactiae with respective cps genes shown as arrows.

FIG. 11a: Structures of pAM-V and pAM-IX used to transform wild-type serotype V and IX GBS strains respectively. cps5M, cps5O and cps5I were cloned into pAM401-p80/t80 to obtain pAM-V. Cloning of cps9M and cps9I in pAM401-p80/t80 was performed to obtain pAM-IX.

FIG. 11b: Schematic structure of pAM-IX-V.

FIG. 12: PS V-IX gives a positive signal indicating that the type V(pAM-IX) strain produces chimeric capsular polysaccharide chains that contain repeating units specifically recognized by both type V- and IX-specific mAbs. Heterologous in-trans expression of cps9M and cps9I allows GBS 2603 (V) to assemble capsular polysaccharides reacting with both type V and IX CPS specific antisera.

FIG. 13: Heterologous in-trans expression of cps5M, cps5O and cps5I allows GBS IT-NI-016 (IX) to assemble capsular polysaccharides reacting with both type IX and V CPS specific antisera

FIG. 14: Comparison of 1H NMR spectra of PS V-IX and PS V-IXb. Spectra are highly similar to that of PS V and PS IX and contain features that are characteristic of both capsular polysaccharides. PS V-IXb spectrum is more similar to that of PS V than PS V-IX.

FIG. 15: The average repeating unit composition of PS V-IX and PS V-IXb has been estimated by DEPT NMR. About 75% of the repeating units of the chimeric type V-IX polysaccharide are type IX while the remaining 25% are type V. About 50% of the repeating units of the chimeric type V-IXb polysaccharide are type IX while the remaining 50% are type V.

FIG. 16: Vectors for PS-Ia-Ib-III or PS-Ia-III production in a serotype Ia background.

FIG. 17: PS V-IXb gives a positive signal indicating that the type V(pAM-IX-V) strain produces 15 chimeric capsular polysaccharide chains that contain repeating units specifically recognized by both type V- and IX-specific mAbs. Combined heterologous in-trans expression of cps9M, cps9I, cps5M, cps5O and cps5I allows GBS 2603 (V) to assemble capsular polysaccharides reacting with both type V and IX CPS specific antisera.

FIG. 18: Sandwich dot-blot analysis of PS V-IX and PS V-IXb

FIG. 19: Competitive ELISA confirmed that PSV-IXb binds to type-specific mAbs with half the efficiency of the native polysaccharides at the same concentration. PSV-IXb seems to be evenly composed by PS V and PS IX RUs.

### DETAILED DESCRIPTION OF THE INVENTION

The invention is based on the capsular saccharides of Streptococcus agalactiae.

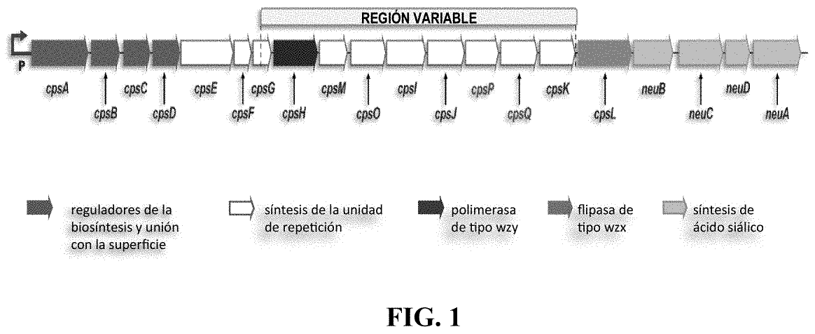

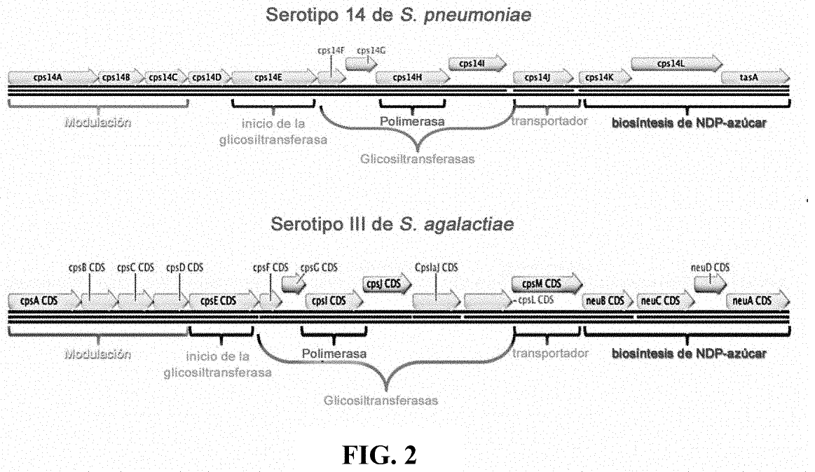

The capsular saccharide of GBS is covalently linked to the peptidoglycan backbone, and is distinct from the group B antigen, which is another saccharide that is attached to the peptidoglycan backbone. All the genes responsible for the synthesis and cell wall attachment of the GBS capsular polysaccharides (CPS) are clustered in the cps operon. This operon is composed of 16-18 genes, the sequences of which differ among serotypes (FIG. 1). The capsular polysaccharide assembly pathway of some Streptococcus pneumoniae serotypes is very similar to that of GBS, as they are both polymerase-dependent. Specifically, within the serotypes that have a polymerase-dependent CPS synthesis machinery, the cps operons have the same organization (FIG. 2). In some S. pneumoniae serotypes even the chemical structure of the CPS is similar to that of some GBS serotypes. For example the chemical structure of the CPS of S. pneumoniae serotype 14 and GBS serotype III is very similar and the aminoacidic sequence identity rate between the homologue proteins encoded by their cps operons is 39.5%. Thus, whilst the invention is described below with a particular emphasis on GBS, the inventors finding are also applicable to the preparation of chimeric polysaccharides of S. pneumoniae.

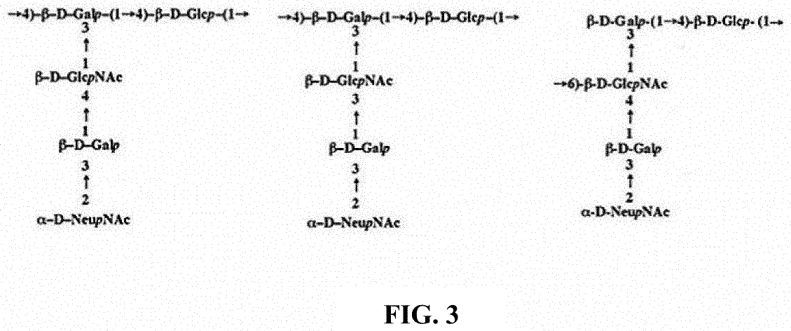

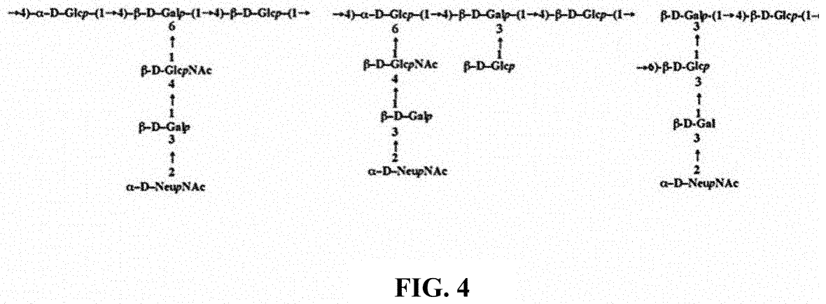

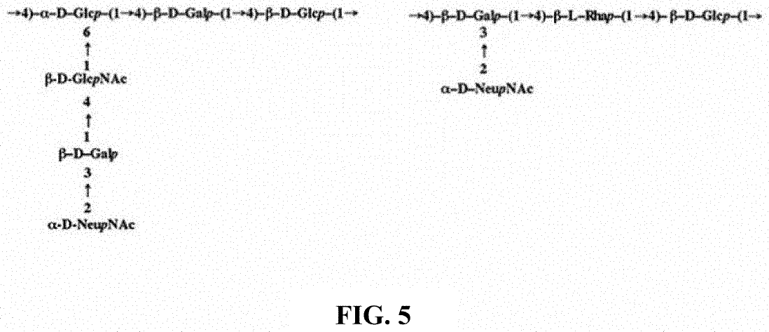

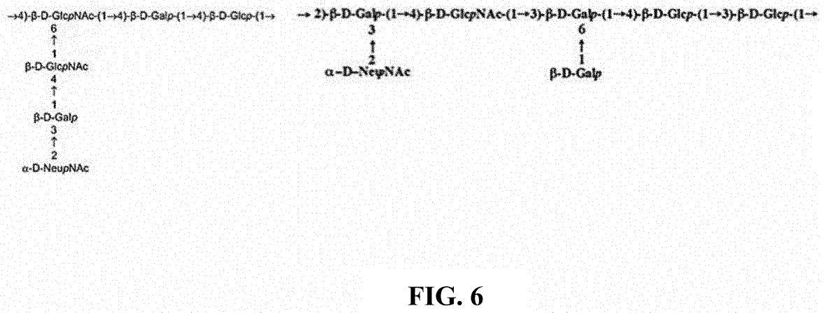

The precise chemical structures of GBS serotypes Ia, Ib, II, III, IV, V, VI, VII, VIII and IX capsular polysaccharides are well described (13-20). They are composed of repeating units of four to seven monosaccharides with a backbone and one or two side chains. Four monosaccharides (i.e. Glcp, Galp, GlcpNAc, and NeupNAc) are present in all ten described serotypes, and the NeupNAc residue is always found at the terminus of one of their side chains. However, the pattern of glycosidic linkages is unique to each serotype. Thus, a subunit or repeating unit (RU) is the part of the capsular polysaccharide whose repetition by linking of the repeating units together successively produces the complete polysaccharide. Particularly, the repeating unit is an oligosaccharide repeating unit. The structures of the RUs of GBS capsular polysaccharides Ia, Ib, III are shown in FIG. 3. The structures of the RUs of GBS capsular polysaccharides IV, V and VI are shown in FIG. 4. The structures of the RUs of GBS capsular polysaccharides VII and VIII are shown in FIG. 5. The structures of the RUs of GBS capsular polysaccharides IX and II are shown in FIG. 6.

The Inventors have discovered that, recombinant GBS strains expressing foreign or exogenous cps genes can produce chimeric capsular polysaccharides. Such chimeric capsular polysaccharides comprise two or more different repeating units having the structure of repeating units from two or more different serotypes. The chimeric capsular polysaccharides can be used to simplify manufacture of multivalent conjugate vaccines.

Chimeric capsular polysaccharides of the invention may also comprise novel epitopes not present in native, wild-type capsular polysaccharides of GBS serotypes Ia, Ib, II, III, IV, V, VI, VII, VIII, IX and Streptococcus pneumoniae serotypes 1, 2, 3, 4, 5, 6b, 7F, 8,9N, 9V, 10A, 11A, 12F, 14, 15B, 17F, 18C, 19F, 19A, 20, 22F, 23F, and 33F. Since the capsular polysaccharide is one of the main targets for vaccination, a major vaccine-related concern for both GBS and S. pneumoniae is the possibility of capsular switching among serotypes. Capsular polysaccharide-based vaccination may exert selective pressure for virulent genotypes to switch capsules and escape vaccine coverage. As a result, the use of such chimeric capsular polysaccharides comprising novel epitopes may be advantageous in preventing or reducing capsular switching by pre-empting the emergence of new capsular serotypes.

### Bacteria

Chimeric capsular polysaccharides of the invention are prepared from Streptococcus agalactiae which express at least one exogenous cps gene due to genetic modification. Thus, provided herein is an engineered Streptococcus agalactiae bacterium for the production of a chimeric capsular polysaccharide, wherein the bacterium comprises an endogenous capsular polysaccharide gene cluster and at least one foreign or exogenous cps gene

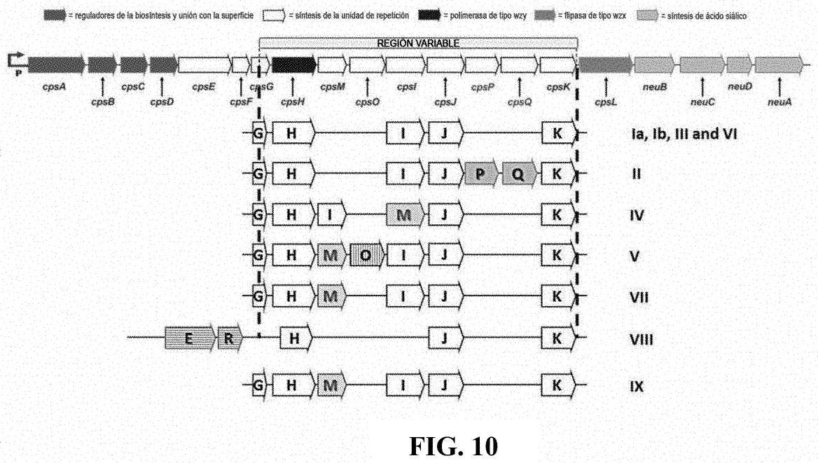

The DNA region of the cps operon is composed of 16 to 18 genes in the different GBS serotypes [149]. The 5′ cpsABCD genes are predicted to be involved in the regulation of capsule synthesis; the central region from cpsE to cpsL encodes the enzymes responsible for the synthesis, transport, and polymerization of the polysaccharide repeating units; finally, neuBCDA genes are responsible for the synthesis of the activated sialic acid, a sugar component present in all GBS capsular polysaccharides [150]. FIG. 10 provides a schematic of the cps operon with respective cps genes. Cps genes derived from serotype V are identified using the nomenclature cps5G, cps5H, cps5M, cps5O and so on. Cps genes derived from serotype IX are identified using the nomenclature cps9G, cps9H, cps9M and so forth. Cps genes derived from other serotypes are identified using similar nomenclature and the identifiers, 1a, 1b, 2, 3, 4, 6, 7 and 8. A similar cps operon exists in Streptococcus pneumoniae.

The term “endogenous” refers to a native gene in its natural location in the genome of an organism. A “foreign” or “exogenous” gene refers to DNA sequences or genes which are not normally present in the cell being transformed, or perhaps are simply not present in the form, structure, etc., as found in the transforming DNA segment or gene, for example, a cps gene not normally found in the serotype of the host cell but that is introduced by gene transfer. Thus, use of the term “exogenous cps gene” in this sense means that the engineered bacterium is of a first serotype, for example, selected from GBS serotypes Ia, Ib, II, III, IV, V, VI, VII, VIII, IX and the at least one exogenous cps gene is from a second, different, serotype. In some embodiments the engineered Streptococcus agalactiae or Streptococcus pneumoniae expresses at least two exogenous cps genes from second and/or third, different, serotypes. The at least one exogenous cps gene may be selected from the group consisting of cpsE, cpsF, cpsG, cpsH, cpsM, cpsO, cpsI, cpsJ, cpsP, cpsQ, cpsK and cpsL. Particular exogenous cps genes include cpsM, cpsO and cpsI. The at least one exogenous cps gene is encoded by an exogenous (e.g., recombinant) nucleic acid, which has been introduced into the engineered Streptococcus agalactiae or Streptococcus pneumoniae cell.

The nucleic acid can be introduced on an expression vector for expression in a Streptococcus agalactiae or Streptococcus pneumoniae cell. The expression vector will generally comprise signals capable of expressing the endogenous cps gene encoded by the introduced nucleic acid. For example, an expression vector may comprise a complete set of control sequences including initiation, promoter and termination sequences which function in a bacterial cell. Suitable expression vectors may comprise 5′ and 3′ regulatory sequences operably linked to the sequences of interest. The nature of any regulatory sequences provided in the expression construct will depend upon the desired expression pattern. Types of regulatory sequences will be known to persons skilled in the art. A vector may also contain one or more restriction sites or homologous recombination sites, to enable insertion of the gene into the host cell genome, at a pre-selected position. In the case of the nucleotide sequence, this may be operably linked to the gene sequence whose expression is to be modified. Also provided on the expression vector may be transcription and translation initiation regions, to enable expression of the incoming genes, transcription and translational termination regions, and regulatory sequences.

In some embodiments the vector is delivered and integrated in the bacterial chromosome by means of homologous and/or site specific recombination. Integrative vectors used to deliver such genes and/or operons can be conditionally replicative or suicide plasmids, bacteriophages, transposons or linear DNA fragments obtained by restriction hydrolysis or PCR amplification. Integration is preferably targeted to chromosomal regions dispensable for growth in vitro. Alternatively, the expression vector can be non-integrative, for example, an episomal vector such as circular/linear replicative plasmids, cosmids, phasmids, lysogenic bacteriophages or bacterial artificial chromosomes. Selection of the recombination event can be selected by means of selectable genetic marker such as genes conferring resistance to antibiotics (for instance kanamycin, erythromycin, chloramphenicol, or gentamycin), genes conferring resistance to heavy metals and/or toxic compounds or genes complementing auxotrophic mutations. Suitable vectors and transformation systems are known in the art and exemplified below.

The cps genes of interest may be encoded in a single expression vector, or in different expression vectors. In the latter case, the different expression vectors may be co-transfected either simultaneously or successively into the Streptococcus agalactiae or Streptococcus pneumoniae cell. “Co-transfection” means the process of transfecting a Streptococcus agalactiae or Streptococcus pneumoniae cell with more than one expression vector. When the cell has been co-transfected with an expression vector capable of expressing one or more first cps genes and a vector capable of expressing one or more second cps genes, the vectors may contain independently selectable markers. Where nucleotide sequences encoding two or more cps genes of interest are contained in a single expression vector, in some embodiments, the nucleotide sequences will be operably linked to a common control element (e.g., a promoter), e. g., the common control element controls expression of all cps gene encoding nucleotide sequences on the single expression vector. In some embodiments, the nucleotide sequences encoding different cps genes are operably linked to different control element(s) (e.g., promoter(s)). In some embodiments, one of the nucleotide sequences may be operably linked to an inducible promoter, and one or more of the other nucleotide sequences may be operably linked to a constitutive promoter.

Competition between alternative repeating units as substrates for the enzymes catalyzing the downstream steps of cps production, might favour the synthesis of the homologous or heterologous repeating unit. Thus, efficient chimeric capsular polysaccharide production may require appropriately balanced expression of the endogenous and exogenous cps genes. In order to balance expression, the skilled person will be aware of a number of options. These may include, by way of non-limiting example, (i) promoter replacement; (ii) gene addition; and/or (iii) gene replacement.

In promoter replacement, the promoter which controls expression of one or more endogenous cps gene may be replaced with a promoter to provide lower or higher levels of expression. A particular promoter for use in the invention is the GBS P80 promoter (Buccato S., et al. (2006) J. Infect. Dis. 194, 331-340). Other promoters are known in the art and include, but are not limited to, a bacteriophage T7 R A polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like. In certain embodiments the cps genes of interest are operably linked to an inducible promoter or to a constitutive promoter. Inducible and constitutive promoters are well known to those of skill in the art.

In gene addition, a bacterium which already expresses the endogenous cps gene receives a second copy of the relevant gene. This second copy can be integrated into the bacterial chromosome or can be on an episomal element such as a plasmid. The effect of the gene addition is to additively increase expression by increasing gene copy number. Where a plasmid is used, it is ideally a plasmid with a high copy number e.g. above 10, or even above 100. In gene replacement, gene addition occurs but is accompanied by deletion of the existing copy of the gene. For instance, at least one endogenous cps gene may be deleted and replaced by a plasmid-encoded copy. Expression from the replacement copy, depending on the promoter used, may be higher or lower than expression from the previous copy. Thus, in some embodiments, the engineered Streptococcus agalactiae or Streptococcus pneumoniae bacterium comprises a deletion or inactivation of one or more genes of the endogenous capsular polysaccharide gene cluster. The one or more deleted genes may comprise one or more of the cpsE, cpsF, cpsG, cpsM, cpsI and cpsJ cpsM, cpsO and cpsI genes. The replacement copy may be an exogenous cps gene and/or a copy of the native, endogenous cps gene.

In some embodiments, more than one event of gene addition or gene replacement may occur such that expression from multiple copies of the cps gene of interest or combinations of over or under expression of different cps genes of interest may take place.

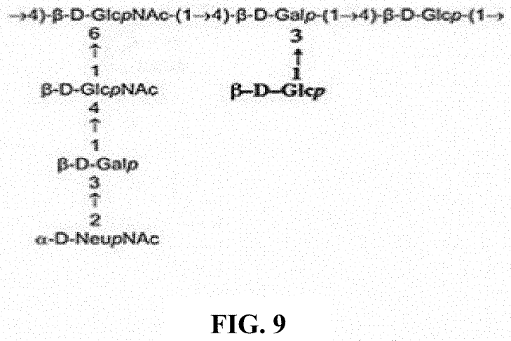

In some embodiments a bacterium expresses an exogenous cpsO gene, particularly a cps5O gene, particularly the bacterium is Streptococcus agalactiae serotype IX. FIG. 9 exemplifies a chimeric serotype IX repeating unit obtained from bacteria expressing cps5O with the additional side chain shown in bold.

In some embodiments a bacterium expresses exogenous cpsM and cpsI genes, particularly cps9M and cps9I genes, particularly the bacterium is Streptococcus agalactiae serotype V.

In some embodiments a bacterium expresses exogenous cpsM, cpsO and cpsI genes, particularly cps5M, cps5O and cps5I genes, particularly the bacterium is Streptococcus agalactiae serotype IX.

In some embodiments, a bacterium produces a chimeric capsular polysaccharide that comprises at least one repeating unit of GBS capsular polysaccharide serotype Ia, at least one repeating unit of GBS capsular polysaccharide serotype Ib and at least one repeating unit of GBS capsular polysaccharide serotype III, wherein the repeating units are joined by glycosidic bonds. Particularly the ratio of the repeating units of Ia, Ib and III is about 1:1:1.

In some embodiments, a bacterium produces a chimeric capsular polysaccharide that comprises at least one repeating unit of GBS capsular polysaccharide serotype V and at least one repeating unit of GBS capsular polysaccharide serotype IX, wherein the repeating units are joined by glycosidic bonds. Particularly the ratio of the repeating units of V to IX is about 1:1.

In some embodiments, a bacterium produces a chimeric capsular polysaccharide that comprises at least one repeating unit of GBS capsular polysaccharide serotype V, at least one repeating unit of GBS capsular polysaccharide serotype IX and at least one repeating unit of GBS capsular polysaccharide serotype VII, wherein the repeating units are joined by glycosidic bonds. Particularly the ratio of the repeating units of V, IX and VII is about 1:1:1.

Particularly reference to ‘at least one repeating unit’ may refer to at least 2, 10, 20, 50, 60, 70, 80, 90, 100 repeating units, at least 150, 200, 250, 500, 1000 or more repeating units. Particularly the ratio of the repeating units is about 1:1 or 1:1:1.

Methods of making the nucleic acid constructs and vectors described herein are well known to those of skill in the art, and specific methods are illustrated in the examples. Cloning and bacterial transformation methods, DNA vectors and the use of regulatory sequences are well known to the skilled artisan and may for instance be found in Current Protocols in Molecular Biology, F. M. Ausubel et al, Wiley Interscience, 2004, incorporated herein by reference.

### The Chimeric Capsular Polysaccharide

Chimeric capsular polysaccharides of the invention comprise two or more different repeating units having the structure of repeating units from two or more different GBS or Streptococcus pneumoniae serotypes. The chimeric capsular polysaccharides of the invention also comprise at least one glycosidic bond or linkage between a molecule in a first repeating unit and a molecule in a second repeating unit. By way of non-limiting example, the molecules are generally carbohydrate molecules such as sugar molecules. The molecule in the first repeating unit may be β-d-Glcp. The molecule in the second repeating unit may be β-d-Galp, β-d-GlcpNAc, β-d-Glcp or α-d-Glcp. Sugar molecules may be joined by a bond between carbon atom number 1 (C1) in one sugar of the first repeating unit and the fourth carbon atom (C4) of one sugar of the second repeating unit in the chimeric capsular polysaccharide, (designated as 1-4), by a bond between carbon atom number 1 (C1) in one sugar of the first repeating unit and the second carbon atom (C2) of one sugar of the second repeating unit in the chimeric capsular polysaccharide (designated as 1→2) or by a bond between carbon atom number 1 (C1) in one sugar of the first repeating unit and the sixth carbon atom (C6) of one sugar of the second repeating unit in the chimeric capsular polysaccharide (designated as 1→6). The use of 1→2 and 1→6 refers to covalent binding between carbon atoms at differently numbered positions in the sugar. Particular examples of different glycosidic linkages occurring between repeating units in capsular polysaccharides include, β-d-Glcp-(1→4)-β-d-Galp, β-d-Glcp-(1→6)-β-d-GlcpNAc, β-d-Glcp-(1→6)-β-d-Glcp, β-d-Glcp-(1→2)-β-d-Galp and β-d-Glcp-(1→4)-α-d-Glcp. In some embodiments, the invention provides chimeric capsular polysaccharides comprising oligosaccharide repeating units joined by at least two different types of glycosidic bond. Particularly chimeric capsular polysaccharides may comprise oligosaccharide repeating units joined by at least two different types of glycosidic bond selected from the group consisting of β-d-Glcp-(1→4)-β-d-Galp, β-d-Glcp-(1→6)-β-d-GlcpNAc, β-d-Glcp-(1→6)-β-d-Glcp, β-d-Glcp-(1→2)-β-d-Galp and β-d-Glcp-(1→4)-α-d-Glcp

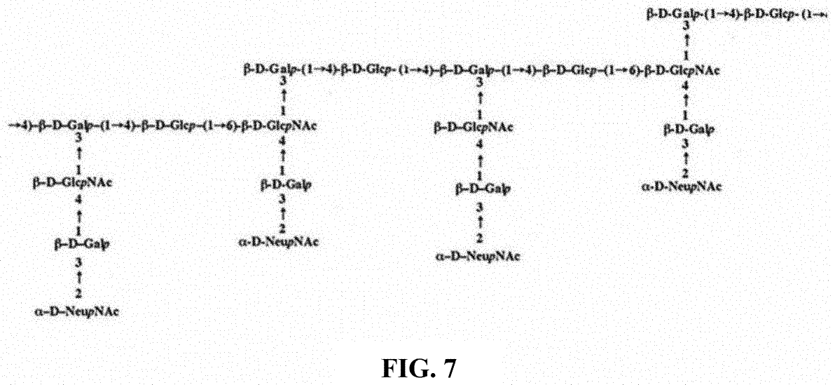

The type Ia and III repeating units of GBS are nearly identical, containing the disaccharide and the variable trisaccharide connected by a 1→3 linkage. The only difference between the wild-type capsular polysaccharides of GBS serotypes Ia and III is the glycosidic bond that connects one repeating unit to the next. In type Ia, the repeating units are linked 1→4 through the β-d-Galp of the disaccharide. In type III the repeating units are joined 1→6 through the β-d-GlcpNAc of the variable trisaccharide. Thus a chimeric capsular polysaccharide of GBS serotypes Ia and III comprises repeating units linked 1→4 through the β-d-Galp of the disaccharide and repeating units linked 1→6 through the β-d-GlcpNAc of the variable trisaccharide. More particularly a chimeric capsular polysaccharide of GBS serotypes Ia and III comprises oligosaccharide RUs joined by β-d-Glcp-(1→4)-β-d-Galp and β-d-Glcp-(1→6)-β-d-GlcpNAc glycosidic bonds. By way of example, a type Ia/III chimeric polysaccharide may comprise type Ia RUs and type III RUs joined by β-d-Glcp-(1→4)-β-d-Galp and β-d-Glcp-(1→6)-β-d-GlcpNAc glycosidic bonds in the arrangement shown in FIG. 7. One skilled in the art will recognise that other arrangements of RUs and bonds are possible.

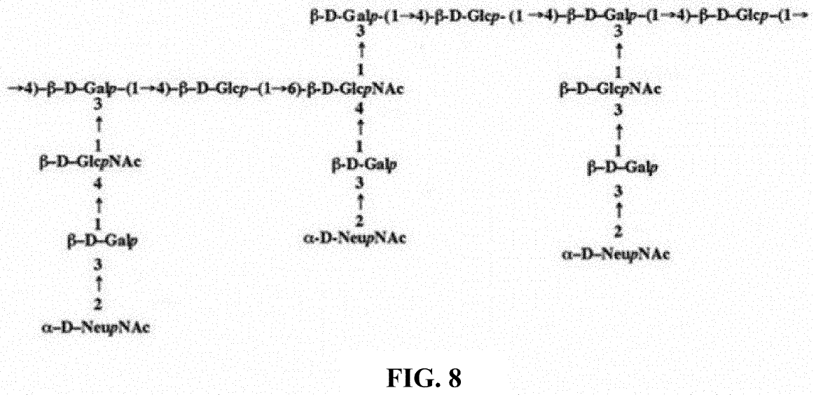

In some embodiments, the invention provides chimeric capsular polysaccharides comprising oligosaccharide RUs joined by at least two, at least three, at least four or at least five different types of glycosidic bond. Particularly the glycosidic bonds are selected from the group consisting of β-d-Glcp-(1→4)-β-d-Galp, β-d-Glcp-(1→6)-β-d-GlcpNAc, β-d-Glcp-(1→6)-β-d-Glcp, β-d-Glcp-(1→2)-β-d-Galp and β-d-Glcp-(1→4)-α-d-Glcp. FIG. 8 provides an example of a type Ia/Ib/III chimeric polysaccharide that comprises type Ia, type III and type Ib RUs joined by β-d-Glcp-(1→4)-β-d-Galp and β-d-Glcp-(1→6)-β-d-GlcpNAc glycosidic bonds. One skilled in the art will recognise that other arrangements of these RUs and bonds are possible.

The ratio of oligosaccharide RUs of the first capsular polysaccharide serotype to oligosaccharide RUs of the second, different capsular polysaccharide serotype may vary. Suitable ratios, for example determined by number or mass, may include 1:1, 1:2, 1:3 1:4, 2:1, 3:1 or 4:1. Generally preferred ratios will be balanced and in the order of 1:1 but the precise ratio may be difficult to control exactly. Alternatively, the content of oligosaccharide RUs of the first capsular polysaccharide serotype and oligosaccharide RUs of the second, different capsular polysaccharide serotype may be presented in terms of percentage (%), for example determined by number or mass. Where chimeric capsular polysaccharides comprise two different types of RU, preferably about 50% of the RUs will be of one type and about 50% of linkages will be of the other type. One skilled in the art will be aware that such percentages will not always be precise and there may be some variation in these figures. For example, there may be about X % of RUs of the first type and about Y % of the second type, wherein Y=100−X, for example, when X=30%, Y=70%; when X=35%, Y=65%; when X=40%, Y=60%, etc. Variances of plus or minus 10%, 11%, 12%, 13%, 14%, 15% could be expected. Where chimeric polysaccharides comprise at least three different types of RU, suitable ratios may include 1:1:1, 1:1:2, 1:1:3, 1:1:4, 1:2:1, 1:3:1, 1:2:2, 1:2:3, 1:2:4, 1:4:1, 1:4:2, 1:4:3, 1:4:4, 2:1:1, 2:1:2, 2:1:3, 2:1:4, 2:2:1, 2:3:1, 2:4:1, 4:1:1, 4:1:2, 4:1:3, 4:1:4, 4:2:1, 4:3:1 and 4:4:1. Similarly, suitable percentages may follow the pattern, X %+Y %+Z %=100%, etc.

When chimeric capsular polysaccharides comprise two different types of glycosidic bond, preferably about 50% of the glycosidic linkages will be of one type and about 50% of linkages will be of the other type. One skilled in the art will be aware that such percentages will not always be precise and there may be some variation in these figures. For example, there may be about X % of glycosidic linkages of one type and about Y % of the other type, wherein Y=100−X. For example, when X=30%, Y=70%; when X=35%, Y=65%; when X=40%, Y=60%, etc.

Chimeric capsular polysaccharides of the invention may be in their native form, or may be modified. For example, polysaccharides may be depolymerised to give shorter fragments for use with the invention e.g. by hydrolysis in mild acid, by heating, by sizing chromatography, etc. Chain length has been reported to affect immunogenicity of GBS saccharides in rabbits [4].

Particularly the chimeric capsular polysaccharide is a high molecular weight polymer. For GBS it is preferred to use chimeric capsular polysaccharides with MW>30 kDa, for example, a MW up to −50 kDa, about 100 kDa, about 140 kDa, about 200 kDa, about 230 kDa, about 260 kDa or any range there between, for example, having a MW in the range of 50-200 kDa, 80-150 kDa, 150-300 kDa, 175-275 kDa or 175-250 kDa. Molecular masses can be measured by gel filtration relative to dextran standards, such as those available from Polymer Standard Service [11].

The chimeric capsular polysaccharides may be chemically modified, for example, the saccharide may be de-O-acetylated (partially or fully), de-N-acetylated (partially or fully), N-propionated (partially or fully), etc. De-acetylation may occur before, during or after conjugation, but preferably occurs before conjugation. Depending on the particular saccharide, de-acetylation may or may not affect immunogenicity. The relevance of 0-acetylation on GBS saccharides in various serotypes is discussed in reference 12, and in some embodiments 0-acetylation of sialic acid residues at positions 7, 8 and/or 9 is retained before, during and after conjugation e.g. by protection/de-protection, by re-acetylation, etc. However, typically the chimeric capsular polysaccharide used in the present invention has substantially no O-acetylation of sialic acid residues at positions 7, 8 and/or 9. In particular, when the chimeric capsular polysaccharide has been purified by base extraction as described below, then 0-acetylation is typically lost (ref 12). The effect of de-acetylation etc. can be assessed by routine assays.

Chimeric capsular polysaccharides can be purified by known techniques, as described in the references such as 2 and 13. A typical process involves base extraction, centrifugation, filtration, RNase/DNase treatment, protease treatment, concentration, size exclusion chromatography, ultrafiltration, anion exchange chromatography, and further ultrafiltration. Treatment of GBS cells with the enzyme mutanolysin, which cleaves the bacterial cell wall to free the cell wall components, is also useful.

As an alternative, the purification process described in reference 14 can be used. This involves base extraction, ethanol/CaCl₂) treatment, CTAB precipitation, and re-solubilisation. A further alternative process is described in reference 15.

Capsular polysaccharides from Streptococcus pneumoniae can be prepared by standard techniques known to those skilled in the art, for example, as disclosed in EP497524 and EP497525.

Chimeric capsular polysaccharides of the invention may also be described or specified in terms of their cross-reactivity. The term “cross-reactive” as used herein refers to the ability of the immune response induced by chimeric capsular polysaccharides of the invention to stimulate the production of antibodies capable of reacting with at least two different GBS or Streptococcus pneumoniae serotypes. The term “cross-protective” as used herein refers to the ability of the immune response, induced by chimeric capsular polysaccharides of the invention, to prevent or attenuate infection or disease by at least two different GBS or Streptococcus pneumoniae serotypes. In particular embodiments of the present disclosure, the chimeric capsular polysaccharides of the present disclosure are cross-reactive and/or cross-protective against a plurality of GBS or Streptococcus pneumoniae serotypes, for example, 2, 3, 4, 5, 6, 7, 8, 9 or 10 serotypes. Cross-reactivity is deemed to be an indicator of cross-protection. It will be readily appreciated by those of skill in the art that the present invention facilitates vaccine manufacture by permitting the production of one chimeric capsular polysaccharide which is capable of offering protection against multiple infectious GBS or Streptococcus pneumoniae serotypes.

### Conjugation of Chimeric Capsular Polysaccharides

Chimeric capsular polysaccharides of the invention may be provided in the form of a conjugate comprising (i) a chimeric capsular polysaccharide and (ii) a carrier protein. Thus in certain embodiments, conjugates comprising (i) a capsular polysaccharide and (ii) a carrier protein, are characterized in that the capsular polysaccharide comprises at least one oligosaccharide RU of a first GBS capsular polysaccharide serotype and at least one oligosaccharide RU of a second GBS capsular polysaccharide serotype and optionally, at least one oligosaccharide RU of a third GBS capsular polysaccharide serotype wherein the oligosaccharide RUs are joined by glycosidic bonds. In other embodiments, conjugates comprising (i) a capsular polysaccharide and (ii) a carrier protein, are characterized in that the capsular polysaccharide comprises at least one oligosaccharide RU of a first Streptococcus pneumoniae capsular polysaccharide serotype and at least one oligosaccharide RU of a second Streptococcus pneumoniae capsular polysaccharide serotype and optionally, at least one oligosaccharide RU of a third Streptococcus pneumoniae capsular polysaccharide serotype wherein the oligosaccharide RUs are joined by glycosidic bonds. In general, covalent conjugation of saccharides to carriers enhances the immunogenicity of saccharides as it converts them from T-independent antigens to T-dependent antigens, thus allowing priming for immunological memory. Conjugation is particularly useful for paediatric vaccines [e.g. ref 16] and is a well known technique [e.g. reviewed in refs. 17 to 25]. Thus the processes of the invention may include the further step of conjugating the purified saccharide to a carrier molecule.

The term “conjugate” refers to a chimeric capsular saccharide linked covalently to a carrier protein. In some embodiments a chimeric capsular saccharide is directly linked to a carrier protein. In other embodiments a chimeric capsular saccharide is indirectly linked to a protein through a spacer or linker. As used herein, the term “directly linked” means that the two entities are connected via a chemical bond, preferably a covalent bond. As used herein, the term “indirectly linked” means that the two entities are connected via a linking moiety (as opposed to a direct covalent bond). In certain embodiments the linker is adipic acid dihydrazide. Representative conjugates in accordance with the present invention include those formed by joining together of the chimeric capsular polysaccharide with the carrier protein.

Covalent linkage of polysaccharides to proteins is known in the art and is generally achieved by targeting the amines of lysines, the carboxylic groups of aspartic/glutamic acids or the sulfhydryls of cysteines. For example, cyanate esters randomly formed from sugar hydroxyls can be reacted with the lysines of the protein or the hydrazine of a spacer which are then condensed to the carboxylic acids of the carrier protein via carbodiimide chemistry. Alternatively, aldehydes generated on purified polysaccharide by random periodate oxidation can either be directly used for reductive amination onto the amines of the carrier protein, or converted into amines for following insertion of a spacer enabling the conjugation step to the protein via thioether or amide bond formation. Glycoconjugates obtained by these methods present complex cross-linked structures. A strategy aimed at simplifying the structure of the final conjugate employs partial hydrolysis of the purified capsular polysaccharide and following fractionation to select an intermediate chain length population. A primary amino group can then be introduced at the oligosaccharide reducing termini to be used finally for insertion of either a diester or a bifunctional linker ready for conjugation to the protein.

The term “carrier protein” refers to a protein to which the chimeric polysaccharide is coupled or attached or conjugated, typically for the purpose of enhancing or facilitating detection of the antigen by the immune system. Capsular polysaccharides are T-independent antigens that are poorly immunogenic and do not lead to long-term protective immune responses. Conjugation of the polysaccharide antigen to a protein carrier changes the context in which immune effector cells respond to polysaccharides. The term carrier protein is intended to cover both small peptides and large polypeptides (>10 kDa). The carrier protein may comprise one or more T-helper epitopes. The peptide may be coupled to the carrier protein by any means such as chemical conjugation.

Useful carrier proteins include bacterial toxins or toxoids, such as diphtheria toxoid or tetanus toxoid. Fragments of toxins or toxoids can also be used e.g. fragment C of tetanus toxoid [26]. The CRM197 mutant of diphtheria toxin [27-29] is a particularly useful with the invention. Other suitable carrier proteins include the N. meningitidis outer membrane protein [30], synthetic peptides [31,32], heat shock proteins [33,34], pertussis proteins [35,36], cytokines [37], lymphokines [37], hormones [37], growth factors [37], human serum albumin (preferably recombinant), artificial proteins comprising multiple human CD4⁺ T cell epitopes from various pathogen-derived antigens [38] such as N19 [39], protein D from Hinfluenzae [40,41], pneumococcal surface protein PspA [42], pneumolysin [43], iron-uptake proteins [44], toxin A or B from C. difficile [45], recombinant Pseudomonas aeruginosa exoprotein A (rEPA) [46], a GBS protein [123], etc.

Particularly suitable carrier proteins include CRM197, tetanus toxoid (TT), tetanus toxoid fragment C, protein D, non-toxic mutants of tetanus toxin and diphtheria toxoid (DT). It has been observed that pre-exposure to the carrier can in some cases lead to reduction of the anti-carbohydrate immune response against glycoconjugate vaccines (carrier epitope suppression). Use of alternatives to DT, TT and CRM197, usually employed in the manufacturing of most glycoconjugate vaccines currently on the market, could be a way to avoid this possibility. Other suitable carrier proteins include protein antigens GBS80, GBS67 and GBS59 from Streptococcus agalactiae. Other suitable carrier proteins include fusion proteins, for example, GBS59(6xD3) disclosed in WO2011/121576 and GBS59(6xD3)-1523 disclosed in EP14179945.2. The use of such GBS protein antigens may be advantageous for a GBS vaccine because, in contrast to heterologous carriers like CRM197, the protein has a dual role increasing immunogenicity of the polysaccharide whilst also provoking a protective immune response. Hence, the immunologic response elicited against the carrier may provide an additional protective immunologic response against GBS, particularly against a GBS protein.

Conjugation of GBS saccharides has been widely reported e.g. see reference 1. The typical prior art process for GBS saccharide conjugation typically involves reductive amination of a purified saccharide to a carrier protein such as tetanus toxoid (TT) or CRM197 [2]. The reductive amination involves an amine group on the side chain of an amino acid in the carrier and an aldehyde group in the saccharide. An aldehyde group may be generated before conjugation by oxidation (e.g. periodate oxidation) of a portion (e.g. between 5 and 40%, particularly between 10 and 30%, preferably about 20%) of the saccharide's sialic acid residues [2,47]. An alternative conjugation process involves the use of —NH₂ groups in the saccharide (either from de-N-acetylation, or after introduction of amines) in conjunction with bifunctional linkers, as described in ref 48. In some embodiments, one or more of the conjugates of the present invention have been prepared in this manner. A further alternative process is described in WO96/40795 and Michon et al. (2006) Clin Vaccine Immunol 2006 August; 13(8):936-43.

Attachment to the carrier is preferably via a —NH₂ group e.g. in the side chain of a lysine residue in a carrier protein, or of an arginine residue, or at the N-terminus. Attachment may also be via a —SH group e.g. in the side chain of a cysteine residue.

Conjugates with a saccharide:protein ratio (w/w) of between 1:5 (i.e. excess protein) and 5:1 (i.e. excess saccharide) are typically used, in particular ratios between 1:5 and 2:1. between about 1:1 to 1:2, particularly about 1:1.3. between about 1:1 to 1:2, particularly about 1:1.3. between about 3:1 to 1:1, particularly about 2:1. about 1:1 to 1:5, particularly about 1:3.3, about 2:1 to 1:1, particularly about 1.1:1. Thus a weight excess of saccharide is typical, particularly with longer saccharide chains.

Compositions may include a small amount of free carrier [49]. When a given carrier protein is present in both free and conjugated form in a composition of the invention, the unconjugated form is preferably no more than 5% of the total amount of the carrier protein in the composition as a whole, and more preferably present at less than 2% by weight.

After conjugation, free and conjugated saccharides can be separated. There are many suitable methods, including hydrophobic chromatography, tangential ultrafiltration, diafiltration etc. [see also refs. 50 & 51, etc.]. A preferred method is described in reference 52.

### Immunogenic Compositions

In one embodiment, the invention provides an immunogenic composition comprising a conjugate that is a chimeric capsular saccharide of the invention conjugated to a carrier protein. The immunogenic compositions may comprise more than one conjugate. Embodiments of the invention may comprise two, three, four, five or six conjugates comprising different types of chimeric capsular polysaccharides.

In certain embodiments, the immunogenic compositions will not comprise any conjugates other than those specifically mentioned. However, in some embodiments, the compositions may comprise other conjugates. The immunogenic compositions may comprise any suitable amount of the chimeric capsular saccharide(s) per unit dose. Suitable amounts of the capsular saccharide(s) may be from 0.1 to 50 μg per unit dose. Typically, each chimeric capsular saccharide is present at an amount from 1 to 30 μg, for example from 2 to 25 μg, and in particular from 5 to 20 μg. Suitable amounts of the chimeric capsular saccharide(s) may include 5, 10 and 20 μg per unit dose.

Methods of administering the immunogenic compositions of the invention are discussed below. Briefly, the immunogenic compositions of the invention may be administered in single or multiple doses. The inventors have found that the administration of a single dose of the immunogenic compositions of the invention is effective. Alternatively, one unit dose followed by a second unit dose may be effective. Typically, the second (or third, fourth, fifth etc.) unit dose is identical to the first unit dose. The second unit dose may be administered at any suitable time after the first unit dose, in particular after 1, 2 or 3 months. Typically, the immunogenic compositions of the invention will be administered intramuscularly, e.g. by intramuscular administration to the thigh or the upper arm as described below.

Immunogenic compositions of the invention may include one or more adjuvants. However, the use of unadjuvanted compositions is also envisaged, for example, it may be advantageous to omit adjuvants in order to reduce potential toxicity. Accordingly, immunogenic compositions that do not contain any adjuvant or that do not contain any aluminium salt adjuvant are envisaged.

### Combinations of Conjugates and Other Antigens

The immunogenic compositions of the invention may comprise one or more further antigens.

The further antigen(s) may comprise further GBS conjugates. The different GBS conjugates may include different types of conjugate from the same GBS serotype and/or conjugates from different GBS serotypes. The composition will typically be produced by preparing separate conjugates (e.g. a different conjugate for each serotype) and then combining the conjugates.

The further antigen(s) may comprise GBS amino acid sequences, as set out below.

The further antigen(s) may comprise antigens from non-GBS pathogens. Thus the compositions of the invention may further comprise one or more non-GBS antigens, including additional bacterial, viral or parasitic antigens. These may be selected from the following:
a protein antigen from N. meningitidis serogroup B, such as those in refs. 53 to 59, with protein ‘287’ (see below) and derivatives (e.g. ‘ΔG287’) being particularly preferred.
an outer-membrane vesicle (OMV) preparation from N. meningitidis serogroup B, such as those disclosed in refs. 60, 61, 62, 63 etc.
a saccharide antigen from N. meningitidis serogroup A, C, W135 and/or Y, such as the oligosaccharide disclosed in ref. 64 from serogroup C or the oligosaccharides of ref. 65.
a saccharide antigen from Streptococcus pneumoniae [e.g. refs. 66-68; chapters 22 & 23 of ref 75].
an antigen from hepatitis A virus, such as inactivated virus [e.g. 69, 70; chapter 15 of ref. 75].
an antigen from hepatitis B virus, such as the surface and/or core antigens [e.g. 70,71; chapter 16 of ref. 75].
an antigen from hepatitis C virus [e.g. 72].
an antigen from Bordetella pertussis, such as pertussis holotoxin (PT) and filamentous haemagglutinin (FHA) from B. pertussis, optionally also in combination with pertactin and/or agglutinogens 2 and 3 [e.g. refs. 73 & 74; chapter 21 of ref. 75].
a diphtheria antigen, such as a diphtheria toxoid [e.g. chapter 13 of ref 75].
a tetanus antigen, such as a tetanus toxoid [e.g. chapter 27 of ref. 75].
a saccharide antigen from Haemophilus influenzae B [e.g. chapter 14 of ref 75]
an antigen from N. gonorrhoeae [e.g. 53, 54, 55].
an antigen from Chlamydia pneumoniae [e.g. 76, 77, 78, 79, 80, 81, 82].
an antigen from Chlamydia trachomatis [e.g. 83].
an antigen from Porphyromonas gingivalis [e.g. 84].
polio antigen(s) [e.g. 85, 86; chapter 24 of ref. 75] such as IPV.
rabies antigen(s) [e.g. 87] such as lyophilised inactivated virus [e.g. 88, RabAvert™].
measles, mumps and/or rubella antigens [e.g. chapters 19, 20 and 26 of ref 75].
influenza antigen(s) [e.g. chapters 17 & 18 of ref 75], such as the haemagglutinin and/or neuraminidase surface proteins.
an antigen from Moraxella catarrhalis [e.g. 89].
an antigen from Streptococcus pyogenes (group A streptococcus) [e.g. 90, 91, 92].
an antigen from Staphylococcus aureus [e.g. 93].

Where a saccharide or carbohydrate antigen is used, it is preferably conjugated to a carrier in order to enhance immunogenicity. Conjugation of Hinfluenzae B, meningococcal and pneumococcal saccharide antigens is well known. Toxic protein antigens may be detoxified where necessary (e.g. detoxification of pertussis toxin by chemical and/or genetic means [74]).

Where a diphtheria antigen is included in the composition a tetanus antigen and at least one pertussis antigen may also be included. Similarly, where a tetanus antigen is included, diphtheria and pertussis antigens may also be included. Similarly, where a pertussis antigen is included diphtheria and tetanus antigens may be included.

Antigens may be adsorbed to an aluminium salt. Where there is more than one conjugate in a composition, not all conjugates need to be adsorbed.

One type of preferred composition includes further antigens from sexually-transmitted pathogens, such as: herpesvirus; N. gonorrhoeae; C. trachomatis; etc. Another type of preferred composition includes further antigens that affect the elderly and/or the immunocompromised, and so the GBS antigens of the invention can be combined with one or more antigens from the following non-GBS pathogens: influenza virus, Enterococcus faecalis, Staphylococcus aureus, Staphylococcus epidermis, Pseudomonas aeruginosa, Legionella pneumophila, Listeria monocytogenes, Neisseria meningitidis, and parainfluenza virus.

Antigens in the composition will typically be present at a concentration of at least 1 μg/ml each. In general, the concentration of any given antigen will be sufficient to elicit an immune response against that antigen.

As an alternative to using proteins antigens in the composition of the invention, nucleic acid encoding the antigen may be used [e.g. refs. 94 to 102]. Protein components of the compositions of the invention may thus be replaced by nucleic acid (preferably DNA e.g. in the form of a plasmid) that encodes the protein.

In practical terms, there may be an upper limit to the number of antigens included in compositions of the invention. The number of antigens (including GBS antigens) in a composition of the invention may be less than 20, less than 19, less than 18, less than 17, less than 16, less than 15, less than 14, less than 13, less than 12, less than 11, less than 10, less than 9, less than 8, less than 7, less than 6, less than 5, less than 4, less than 3, or less than 2. The number of GBS antigens in a composition of the invention may be less than 6, less than 5, less than 4, less than 3, or less than 2.

### Pharmaceutical Methods and Uses

The immunogenic compositions of the invention may further comprise a pharmaceutically acceptable carrier. Typical ‘pharmaceutically acceptable carriers’ include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sucrose [103], trehalose [104], lactose, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in reference 105.

Compositions of the invention may be in aqueous form (i.e. solutions or suspensions) or in a dried form (e.g. lyophilised). If a dried vaccine is used then it will be reconstituted into a liquid medium prior to injection. Lyophilisation of conjugate vaccines is known in the art e.g. the Menjugate™ product is presented in lyophilised form. When the immunogenic compositions of the invention include conjugates comprising more than one type of chimeric capsular saccharide, it is typical for the conjugates to be prepared separately, mixed and then lyophilised. In this way, lyophilised compositions comprising two, three or four etc. conjugates as described herein may be prepared. To stabilise conjugates during lyophilisation, it may be preferred to include a sugar alcohol (e.g. mannitol) and/or a disaccharide (e.g. sucrose or trehalose) e.g. at between 1 mg/ml and 30 mg/ml (e.g. about 25 mg/ml) in the composition. The use of sucrose has been recommended as a stabiliser for GBS conjugate vaccines (ref 106). However, it is typical for the stabiliser of the present invention to be mannitol. When the dried vaccine is reconstituted into a liquid medium prior to injection, the concentration of residual mannitol will typically be about 2-20 mg/ml, e.g. 3.75 mg/ml, 7.5 mg/ml or 15 mg/ml. The use of mannitol is advantageous because mannitol is chemically distinct from the monosaccharide repeating units of the GBS capsular saccharides. This means that detection of the capsular saccharides, e.g. for quality control analysis, can be based on the presence of the repeating units of the saccharides without interference from the mannitol. In contrast, a stabiliser like sucrose contains glucose, which may interfere with the detection of glucose repeating units in the saccharides.

Compositions may be presented in vials, or they may be presented in ready-filled syringes. The syringes may be supplied with or without needles. A syringe will include a single dose of the composition, whereas a vial may include a single dose or multiple doses.

Aqueous compositions of the invention are also suitable for reconstituting other vaccines from a lyophilised form. Where a composition of the invention is to be used for such extemporaneous reconstitution, the invention provides a kit, which may comprise two vials, or may comprise one ready-filled syringe and one vial, with the contents of the syringe being used to reactivate the contents of the vial prior to injection.

Compositions of the invention may be packaged in unit dose form or in multiple dose form. For multiple dose forms, vials are preferred to pre-filled syringes. Effective dosage volumes can be routinely established, but a typical human dose of the composition has a volume of 0.5 ml e.g. for intramuscular injection.

The pH of the composition is preferably between 6 and 8, preferably about 7. Stable pH may be maintained by the use of a buffer. The immunogenic compositions of the invention typically comprise a potassium dihydrogen phosphate buffer. The potassium dihydrogen phosphate buffer may comprise about 1-10 mM potassium dihydrogen phosphate, e.g. 1.25 mM, 2.5 mM or 5.0 mM. If a composition comprises an aluminium hydroxide salt, it is preferred to use a histidine buffer [107]. The composition may be sterile and/or pyrogen-free. Compositions of the invention may be isotonic with respect to humans.

Compositions of the invention are immunogenic, and are more preferably vaccine compositions. Vaccines according to the invention may either be prophylactic (i.e. to prevent infection) or therapeutic (i.e. to treat infection), but will typically be prophylactic Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

Within each dose, the quantity of an individual saccharide antigen will generally be between 0.1-50 μg (measured as mass of saccharide), particularly between 1-50 μg or 0.5-25 μg, more particularly 2.5-7.5 μg, e.g. about 1 μg, about 2.5 μg, about 5 μg, about 10 μg, about 15 μg, about 20 μg or about 25 μg. Within each dose, the total quantity of chimeric capsular saccharides will generally be ≤70 μg (measured as mass of saccharide), e.g. ≤60 μg. In particular, the total quantity may be ≤40 μg (e.g. ≤30 μg) or ≤20 μg (e.g. ≤15 μg). It may be advantageous to minimise the total quantity of chimeric capsular saccharide(s) per unit dose in order to reduce potential toxicity. Accordingly, a total quantity of ≤20 μg may be used, e.g. ≤15 μg, ≤7.5 μg or ≤1.5 μg.

GBS and Streptococcus pneumoniae affect various areas of the body and so the compositions of the invention may be prepared in various forms. For example, the compositions may be prepared as injectables, either as liquid solutions or suspensions. The composition may be prepared for pulmonary administration e.g. as an inhaler, using a fine powder or a spray. The composition may be prepared as a suppository or pessary. The composition may be prepared for nasal, aural or ocular administration e.g. as spray, drops, gel or powder [e.g. refs 108 & 109]. Success with nasal administration of pneumococcal saccharides [110,111], Hib saccharides [112], MenC saccharides [113], and mixtures of Hib and MenC saccharide conjugates [114] has been reported.

Compositions of the invention may include an antimicrobial, particularly when packaged in multiple dose format. Compositions of the invention may comprise detergent e.g. a Tween (polysorbate), such as Tween 80. Detergents are generally present at low levels e.g. <0.01%. Compositions of the invention may include sodium salts (e.g. sodium chloride) to give tonicity. A concentration of 10+2 mg/ml NaCl is typical. In some embodiments, a concentration of 4-10 mg/ml NaCl may be used, e.g. 9.0, 7.0, 6.75 or 4.5 mg/ml. Compositions of the invention will generally include a buffer. A phosphate buffer is typical. Compositions of the invention may be administered in conjunction with other immunoregulatory agents. In particular, compositions may include one or more adjuvants. Such adjuvants are known in the art and include, but are not limited to aluminium salts such as alum and MF59.

### Methods of Treatment

The invention also provides a method for raising an immune response in a suitable mammal, comprising administering a pharmaceutical composition of the invention to the mammal. The immune response is preferably protective and preferably involves antibodies. More particularly, the immune response is protective against at least two different GBS serotypes and preferably involves antibodies against at least two GBS serotypes respectively. The method may raise a booster response.

The suitable mammal is preferably a human. Where the vaccine is for prophylactic use, the human is preferably a child (e.g. a toddler or infant) or a teenager; where the vaccine is for therapeutic use, the human is preferably an adult. A vaccine intended for children may also be administered to adults e.g. to assess safety, dosage, immunogenicity, etc. A preferred class of humans for treatment are females of child-bearing age (e.g. teenagers and above). Another preferred class is pregnant females. Elderly patients (e.g. those above 50, 60, 70, 80 or 90 etc. years of age, particularly over 65 years of age), especially those living in nursing homes where the risk of GBS infection may be increased ([115]), are another preferred class of humans for treatment. Women with undetectable level(s) of antibodies against GBS capsular saccharide(s) may have higher rates of GBS infection in their newborns. This is because higher levels of maternal antibodies against GBS capsular saccharides are correlated with reduced risk of disease in newborns [refs. 116 and 117]. Accordingly, administration to these women is specifically envisaged in the present invention.

The invention also provides a composition of the invention for use as a medicament, for example, a vaccine. The medicament is preferably able to raise an immune response in a suitable mammal (i.e. it is an immunogenic composition) and is more preferably a vaccine. The invention also provides the use of a composition of the invention in the manufacture of a medicament for raising an immune response in a suitable mammal.

These uses and methods may be for the prevention and/or treatment of a disease caused by S. agalactiae e.g. neonatal sepsis or bacteremia, neonatal pneumonia, neonatal meningitis, endometritis, osteomyelitis, septic arthritis, etc. These uses and methods may be for the prevention and/or treatment of a disease caused by Spneumoniae, for example, bronchitis, rhinitis, acute sinusitis, otitis media, conjunctivitis, meningitis, bacteremia, sepsis, osteomyelitis, septic arthritis, endocarditis, peritonitis, pericarditis, cellulitis, and brain abscess.

The subject in which disease is prevented may not be the same as the subject that receives the conjugate of the invention. For instance, a conjugate may be administered to a female (before or during pregnancy) in order to protect offspring (so-called ‘maternal immunisation’ [118-120]).

One way of checking efficacy of therapeutic treatment involves monitoring GBS infection after administration of the composition of the invention. One way of checking efficacy of prophylactic treatment involves monitoring immune responses against the, for example, GBS antigens after administration of the composition.

Preferred compositions of the invention can confer an antibody titre in a patient that is superior to the criterion for seroprotection for each antigenic component for an acceptable percentage of human subjects. Antigens with an associated antibody titre above which a host is considered to be seroconverted against the antigen are well known, and such titres are published by organisations such as WHO. Preferably more than 80% of a statistically significant sample of subjects is seroconverted, more preferably more than 90%, still more preferably more than 93% and most preferably 96-100%.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is 0.5 ml. The invention may be used to elicit systemic and/or mucosal immunity.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

### GBS Protein Antigens

As mentioned above, for protection against GBS, GBS proteins can be included in compositions of the invention. These may be used as carrier proteins for conjugates of the invention, carrier proteins for other conjugates, or as unconjugated protein antigens.

GBS protein antigens for use with the invention include those disclosed in references 90 and 121-123. Two particular GBS protein antigens for use with the invention are known as: GBS67; and GBS80 [see ref 90]. A further preferred GBS protein antigen for use with the invention is known as Spb1 [see ref 124]. Particular GBS fusion proteins for use in the invention include GBS59(6xD3) and GBS59(6xD3)-1523. Further details of these antigens are given below.

The sequences for these proteins are provided in SEQ ID NOs 1 to 22 herein. Compositions of the invention may thus include (a) a polypeptide comprising an amino acid sequence selected from SEQ ID NOs 1 to 22, and/or (b) a polypeptide comprising (i) an amino acid sequence that has sequence identity to one or more of SEQ ID NOs 1 to 22 and/or (ii) a fragment of SEQ ID NOs 1 to 22.

Compositions of the invention may also comprise mixtures of these GBS protein antigens.

In particular, compositions of the invention may include:

(a₁) a polypeptide comprising an amino acid sequence of SEQ ID NO 1, and/or (b₁) a polypeptide comprising (i) an amino acid sequence that has sequence identity to SEQ ID NO 1 and/or (ii) a fragment of SEQ ID NO 1;
(a₂) a polypeptide comprising an amino acid sequence of SEQ ID NO 7, and/or (b₂) a polypeptide comprising (i) an amino acid sequence that has sequence identity to SEQ ID NO 7 and/or (ii) a fragment of SEQ ID NO 7; and
(a₃) a polypeptide comprising an amino acid sequence of SEQ ID NO 13, and/or (b₃) a polypeptide comprising (i) an amino acid sequence that has sequence identity to SEQ ID NO 13 and/or (ii) a fragment of SEQ ID NO 13; and
(a₄) a polypeptide comprising an amino acid sequence of SEQ ID NO 17, and/or (b₃) a polypeptide comprising (i) an amino acid sequence that has sequence identity to SEQ ID NO 17; and
(a₅) a polypeptide comprising an amino acid sequence of SEQ ID NO 22, and/or (b₃) a polypeptide comprising (i) an amino acid sequence that has sequence identity to SEQ ID NO 22.

Depending on the particular SEQ ID NO, the degree of sequence identity in (i) is preferably greater than 50% (e.g. 60%, 70%, 75%, 80%, 85%, 90%, 91%, 92%, 93%, 94%, 95%, 96%, 97%, 98%, 99% or more). These polypeptides include homologs, orthologs, allelic variants and functional mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty=12 and gap extension penalty=1.

Depending on the particular SEQ ID NO, the fragments of (ii) should comprise at least n consecutive amino acids from the sequences and, depending on the particular sequence, n is 7 or more (e.g. 8, 10, 12, 14, 16, 18, 20, 22, 24, 26, 28, 30, 35, 40, 45, 50, 60, 70, 80, 90, 100 or more). The fragment may comprise at least one T-cell or, preferably, a B-cell epitope of the sequence. T- and B-cell epitopes can be identified empirically (e.g. using PEPSCAN [125,126] or similar methods), or they can be predicted (e.g. using the Jameson-Wolf antigenic index [127], matrix-based approaches [128], TEPITOPE [129], neural networks [130], OptiMer & EpiMer [131, 132], ADEPT [133], Tsites [134], hydrophilicity [135], antigenic index [136] or the methods disclosed in reference 137 etc.). Removal of one or more domains, such as the N-terminal signal peptide, a leader or signal sequence region, a transmembrane region, a cytoplasmic region or a cell wall anchoring motif can be used.

These polypeptides may, compared to SEQ ID NOs 1 to 22, include one or more (e.g. 1, 2, 3, 4, 5, 6, 7, 8, 9, 10, etc.) conservative amino acid replacements i.e. replacements of one amino acid with another which has a related side chain Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cystine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may also include one or more (e.g. 1, 2, 3, 4, 5, 6, 7, 8, 9, 10, etc.) single amino acid deletions relative to SEQ ID NOs 1 to 22. The polypeptides may also include one or more (e.g. 1, 2, 3, 4, 5, 6, 7, 8, 9, 10, etc.) insertions (e.g. each of 1, 2, 3, 4 or 5 amino acids) relative to the SEQ ID NOs 1 to 22.

Polypeptides of the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis [138,139]. Solid-phase peptide synthesis is particularly preferred, such as methods based on tBoc or Fmoc [140] chemistry. Enzymatic synthesis [141] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [142]. Where D-amino acids are included, however, it is preferred to use chemical synthesis. Polypeptides of the invention may have covalent modifications at the C-terminus and/or N-terminus.

If these GBS proteins are included in compositions of the invention then they can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.). They are preferably used in purified or substantially purified form i.e. substantially free from other polypeptides (e.g. free from naturally-occurring polypeptides), particularly from other GBS or host cell polypeptides).

### GBS67

Nucleotide and amino acid sequence of GBS67 sequenced from serotype V strain 2603 V/R are set forth in ref 90 as SEQ ID NOs 3745 & 3746. The amino acid sequence is SEQ ID NO:1 herein:

MRKYQKFSKILTLSLFCLSQIPLNTNVLGESTVPENGAKGKLVVKKTDDQ
NKPLSKATFVLKTTAHPESKIEKVTAELTGEATFDNLIPGDYTLSEETAP
EGYKKTNQTWQVKVESNGKTTIQNSGDKNSTIGQNQEELDKQYPPTGIYE
DTKESYKLEHVKGSVPNGKSEAKAVNPYSSEGEHIREIPEGTLSKRISEV
GDLAHNKYKIELTVSGKTIVKPVDKQKPLDVVFVLDNSNSMNNDGPNFQR
HNKAKKAAEALGTAVKDILGANSDNRVALVTYGSDIFDGRSVDVVKGFKE
DDKYYGLQTKFTIQTENYSHKQLTNNAEEIIKRIPTEAPKAKWGSTTNGL
TPEQQKEYYLSKVGETFTMKAFMEADDILSQVNRNSQKIIVHVTDGVPTR
SYAINNFKLGASYESQFEQMKKNGYLNKSNFLLTDKPEDIKGNGESYFLF
PLDSYQTQIISGNLQKLHYLDLNLNYPKGTIYRNGPVKEHGTPTKLYINS
LKQKNYDIFNFGIDISGFRQVYNEEYKKNQDGTFQKLKEEAFKLSDGEIT
ELMRSFSSKPEYYTPIVTSADTSNNEILSKIQQQFETILTKENSIVNGTI
EDPMGDKINLQLGNGQTLQPSDYTLQGNDGSVMKDGIATGGPNNDGGILK
GVKLEYIGNKLYVRGLNLGEGQKVTLTYDVKLDDSFISNKFYDTNGRTTL
NPKSEDPNTLRDFPIPKIRDVREYPTITIKNEKKLGEIEFIKVDKDNNKL
LLKGATFELQEFNEDYKLYLPIKNNNSKVVTGENGKISYKDLKDGKYQLI
EAVSPEDYQKITNKPILTFEVVKGSIKNIIAVNKQISEYHEEGDKHLITN
THIPPKGIIPMTGGKGILSFILIGGAMMSIAGGIYIWKRYKKSSDM
SIKKD

GBS67 contains a C-terminus transmembrane region which is indicated by the underlined region closest to the C-terminus of SEQ ID NO: 1 above. One or more amino acids from the transmembrane region may be removed, or the amino acid may be truncated before the transmembrane region. An example of such a GBS67 fragment is set forth below as SEQ ID NO: 2.

MRKYQKFSKILTLSLFCLSQIPLNTNVLGESTVPENGAKGKLVVKKTDDQ
NKPLSKATFVLKTTAHPESKIEKVTAELTGEATFDNLIPGDYTLSEETAP
EGYKKTNQTWQVKVESNGKTTIQNSGDKNSTIGQNQEELDKQYPPTGIYE
DTKESYKLEHVKGSVPNGKSEAKAVNPYSSEGEHIREIPEGTLSKRISEV
GDLAHNKYKIELTVSGKTIVKPVDKQKPLDVVFVLDNSNSMNNDGPNFQR
HNKAKKAAEALGTAVKDILGANSDNRVALVTYGSDIFDGRSVDVVKGFKE
DDKYYGLQTKFTIQTENYSHKQLTNNAEEIIKRIPTEAPKAKWGSTTNGL
TPEQQKEYYLSKVGETFTMKAFMEADDILSQVNRNSQKIIVHVTDGVPTR
SYAINNFKLGASYESQFEQMKKNGYLNKSNFLLTDKPEDIKGNGESYFLF
PLDSYQTQIISGNLQKLHYLDLNLNYPKGTIYRNGPVKEHGTPTKLYINS
LKQKNYDIFNFGIDISGFRQVYNEEYKKNQDGTFQKLKEEAFKLSDGEIT
ELMRSFSSKPEYYTPIVTSADTSNNEILSKIQQQFETILTKENSIVNGTI
EDPMGDKINLQLGNGQTLQPSDYTLQGNDGSVMKDGIATGGPNNDGGILK
GVKLEYIGNKLYVRGLNLGEGQKVTLTYDVKLDDSFISNKFYDTNGRTTL
NPKSEDPNTLRDFPIPKIRDVREYPTITIKNEKKLGEIEFIKVDKDNNKL
LLKGATFELQEFNEDYKLYLPIKNNNSKVVTGENGKISYKDLKDGKYQLI
EAVSPEDYQKITNKPILTFEVVKGSIKNIIAVNKQISEYHEEGDKHLITN
THIPPKGIIPMTGGKGILS

GBS67 contains an amino acid motif indicative of a cell wall anchor, shown in italics in SEQ ID NO: 1 above. In some recombinant host cell systems, it may be preferable to remove this motif to facilitate secretion of a recombinant GBS67 protein from the host cell. Accordingly, in one preferred fragment of GBS67 for use in the invention, the transmembrane and the cell wall anchor motif are removed from GBS67. An example of such a GBS67 fragment is set forth below as SEQ ID NO: 3.

MRKYQKFSKILTLSLFCLSQIPLNTNVLGESTVPENGAKGKLVVKKTDDQ
NKPLSKATFVLKTTAHPESKIEKVTAELTGEATFDNLIPGDYTLSEETAP
EGYKKTNQTWQVKVESNGKTTIQNSGDKNSTIGQNQEELDKQYPPTGIYE
DTKESYKLEHVKGSVPNGKSEAKAVNPYSSEGEHIREIPEGTLSKRISEV
GDLAHNKYKIELTVSGKTIVKPVDKQKPLDVVFVLDNSNSMNNDGPNFQR
HNKAKKAAEALGTAVKDILGANSDNRVALVTYGSDIFDGRSVDVVKGFKE
DDKYYGLQTKFTIQTENYSHKQLTNNAEEIIKRIPTEAPKAKWGSTTNGL
TPEQQKEYYLSKVGETFTMKAFMEADDILSQVNRNSQKIIVHVTDGVPTR
SYAINNFKLGASYESQFEQMKKNGYLNKSNFLLTDKPEDIKGNGESYFLF
PLDSYQTQIISGNLQKLHYLDLNLNYPKGTIYRNGPVKEHGTPTKLYINS
LKQKNYDIFNFGIDISGFRQVYNEEYKKNQDGTFQKLKEEAFKLSDGEIT
ELMRSFSSKPEYYTPIVTSADTSNNEILSKIQQQFETILTKENSIVNGTI
EDPMGDKINLQLGNGQTLQPSDYTLQGNDGSVMKDGIATGGPNNDGGILK
GVKLEYIGNKLYVRGLNLGEGQKVTLTYDVKLDDSFISNKFYDTNGRTTL
NPKSEDPNTLRDFPIPKIRDVREYPTITIKNEKKLGEIEFIKVDKDNNKL
LLKGATFELQEFNEDYKLYLPIKNNNSKVVTGENGKISYKDLKDGKYQLI
EAVSPEDYQKITNKPILTFEVVKGSIKNIIAVNKQISEYHEEGDKHLITN
THIPPKGI

Alternatively, in some recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Three pilin motifs, containing conserved lysine residues have been identified in GBS67. Conserved lysine residues are at amino acid residues 478 and 488, at amino acid residues 340 and 342, and at amino acid residues 703 and 717. The pilin sequences, in particular the conserved lysine residues, are thought to be important for the formation of oligomeric, pilus-like structures of GBS67. Preferred fragments of GBS 67 include at least one conserved lysine residue. Two E boxes containing conserved glutamic residues have also been identified in GBS67. Preferred fragments of GBS 67 include at least one conserved glutamic acid residue. GBS67 contains several regions predicted to form alpha helical structures. Such alpha helical regions are likely to form coiled-coil structures and may be involved in oligomerization of GBS67. GBS67 also contains a region which is homologous to the Cna_B domain of the S. aureus collagen-binding surface protein (pfam05738). This may form a beta sandwich structure. GBS67 contains a region which is homologous to a von Willebrand factor (vWF) type A domain.

The amino acid sequence of GBS67 sequenced from serotype Ib strain H36B is set forth in ref. 143 as SEQ ID NO 20906. The amino acid sequence is SEQ ID NO: 4 herein:

MRKYQKFSKILTLSLFCLSQIPLNTNVLGESTVPENGAKGKLVVKKTDDQ
NKPLSKATFVLKPTSHSESKVEKVTTEVTGEATFDNLTPGDYTLSEETAP
EGYKKTTQTWQVKVESNGKTTIQNSDDKKSIIEQRQEELDKQYPLTGAYE
DTKESYNLEHVKNSIPNGKLEAKAVNPYSSEGEHIREIQEGTLSKRISEV
NDLDHNKYKIELTVSGKSIIKTINKDEPLDVVFVLDNSNSMKNNGKNNKA
KKAGEAVETIIKDVLGANVENRAALVTYGSDIFDGRTVKVIKGFKEDPYY
GLETSFTVQTNDYSYKKFTNIAADIIKKIPKEAPEAKWGGTSLGLTPEKK
REYDLSKVGETFTMKAFMEADTLLSSIQRKSRKIIVHLTDGVPTRSYAIN
SFVKGSTYANQFERIKEKGYLDKNNYFITDDPEKIKGNGESYFLFPLDSY
QTQIISGNLQKLHYLDLNLNYPKGTIYRNGPVREHGTPTKLYINSLKQKN
YDIFNFGIDISGFRQVYNEDYKKNQDGTFQKLKEEAFELSDGEITELMNS
FSSKPEYYTPIVTSADVSNNEILSKIQQQFEKILTKENSIVNGTIEDPMG
DKINLHLGNGQTLQPSDYTLQGNDGSIMKDSIATGGPNNDGGILKGVKLE
YIKNKLYVRGLNLGEGQKVTLTYDVKLDDSFISNKFYDTNGRTTLNPKSE
EPDTLRDFPIPKIRDVREYPTITIKNEKKLGEIEFTKVDKDNNKLLLKGA
TFELQEFNEDYKLYLPIKNNNSKVVTGENGKISYKDLKDGKYQLIEAVSP
KDYQKITNKPILTFEVVKGSIQNIIAVNKQISEYHEEGDKHLITNTHIPP
KGIIPMTGGKGILSFILIGGAMMSIAGGIYIWKRHKKSSDASIEKD

In some embodiments, this variant of GBS67 may be used. Accordingly, where embodiments of the present invention are defined herein by reference to SEQ ID NO: 1, the references to SEQ ID NO: 1 may be substituted by references to SEQ ID NO: 4.

Like GBS67 sequenced from serotype V strain 2603 V/R, GBS67 sequenced from serotype Ib strain H36B contains a C-terminus transmembrane region which is indicated by the underlined region closest to the C-terminus of SEQ ID NO: 2 above. One or more amino acids from the transmembrane region may be removed, or the amino acid may be truncated before the transmembrane region. An example of such a GBS67 fragment is set forth below as SEQ ID NO: 5.

MRKYQKFSKILTLSLFCLSQIPLNTNVLGESTVPENGAKGKLVVKKTDDQ
NKPLSKATFVLKPTSHSESKVEKVTTEVTGEATFDNLTPGDYTLSEETAP
EGYKKTTQTWQVKVESNGKTTIQNSDDKKSIIEQRQEELDKQYPLTGAYE
DTKESYNLEHVKNSIPNGKLEAKAVNPYSSEGEHIREIQEGTLSKRISEV
NDLDHNKYKIELTVSGKSIIKTINKDEPLDVVFVLDNSNSMKNNGKNNKA
KKAGEAVETIIKDVLGANVENRAALVTYGSDIFDGRTVKVIKGFKEDPYY
GLETSFTVQTNDYSYKKFTNIAADIIKKIPKEAPEAKWGGTSLGLTPEKK
REYDLSKVGETFTMKAFMEADTLLSSIQRKSRKIIVHLTDGVPTRSYAIN
SFVKGSTYANQFERIKEKGYLDKNNYFITDDPEKIKGNGESYFLFPLDSY
QTQIISGNLQKLHYLDLNLNYPKGTIYRNGPVREHGTPTKLYINSLKQKN
YDIFNFGIDISGFRQVYNEDYKKNQDGTFQKLKEEAFELSDGEITELMNS
FSSKPEYYTPIVTSADVSNNEILSKIQQQFEKILTKENSIVNGTIEDPMG
DKINLHLGNGQTLQPSDYTLQGNDGSIMKDSIATGGPNNDGGILKGVKLE
YIKNKLYVRGLNLGEGQKVTLTYDVKLDDSFISNKFYDTNGRTTLNPKSE
EPDTLRDFPIPKIRDVREYPTITIKNEKKLGEIEFTKVDKDNNKLLLKGA
TFELQEFNEDYKLYLPIKNNNSKVVTGENGKISYKDLKDGKYQLIEAVSP
KDYQKITNKPILTFEVVKGSIQNIIAVNKQISEYHEEGDKHLITNTHIPP
KGIIPMTGGKGILS

Like GBS67 sequenced from serotype V strain 2603 V/R, GBS67 sequenced from serotype Ib strain H36B contains an amino acid motif indicative of a cell wall anchor, shown in italics in SEQ ID NO: 4 above. Accordingly, in one preferred fragment of GBS67 for use in the invention, the transmembrane and the cell wall anchor motif are removed from GBS67. An example of such a GBS67 fragment is set forth below as SEQ ID NO: 6.

MRKYQKFSKILTLSLFCLSQIPLNTNVLGESTVPENGAKGKLVVKKTDDQ
NKPLSKATFVLKPTSHSESKVEKVTTEVTGEATFDNLTPGDYTLSEETAP
EGYKKTTQTWQVKVESNGKTTIQNSDDKKSIIEQRQEELDKQYPLTGAYE
DTKESYNLEHVKNSIPNGKLEAKAVNPYSSEGEHIREIQEGTLSKRISEV
NDLDHNKYKIELTVSGKSIIKTINKDEPLDVVFVLDNSNSMKNNGKNNKA
KKAGEAVETIIKDVLGANVENRAALVTYGSDIFDGRTVKVIKGFKEDPYY
GLETSFTVQTNDYSYKKFTNIAADIIKKIPKEAPEAKWGGTSLGLTPEKK
REYDLSKVGETFTMKAFMEADTLLSSIQRKSRKIIVHLTDGVPTRSYAIN
SFVKGSTYANQFERIKEKGYLDKNNYFITDDPEKIKGNGESYFLFPLDSY
QTQIISGNLQKLHYLDLNLNYPKGTIYRNGPVREHGTPTKLYINSLKQKN
YDIFNFGIDISGFRQVYNEDYKKNQDGTFQKLKEEAFELSDGEITELMNS
FSSKPEYYTPIVTSADVSNNEILSKIQQQFEKILTKENSIVNGTIEDPMG
DKINLHLGNGQTLQPSDYTLQGNDGSIMKDSIATGGPNNDGGILKGVKLE
YIKNKLYVRGLNLGEGQKVTLTYDVKLDDSFISNKFYDTNGRTTLNPKSE
EPDTLRDFPIPKIRDVREYPTITIKNEKKLGEIEFTKVDKDNNKLLLKGA
TFELQEFNEDYKLYLPIKNNNSKVVTGENGKISYKDLKDGKYQLIEAVSP
KDYQKITNKPILTFEVVKGSIQNIIAVNKQISEYHEEGDKHLITNTHIPP
KGI

### GBS80

GBS80 refers to a putative cell wall surface anchor family protein. Nucleotide and amino acid sequence of GBS80 sequenced from serotype V isolated strain 2603 V/R are set forth in ref 90 as SEQ ID NOs 8779 & 8780. The amino acid sequence is set forth below as SEQ ID NO: 7:

MKLSKKLLFSAAVLTMVAGSTVEPVAQFATGMSIVRAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKTLNRDEHYTIDEPTVDNQNTLKITFKPEKFK
EIAELLKGMTLVKNQDALDKATANTDDAAFLEIPVASTINEKAVLGKAIE
NTFELQYDHTPDKADNPKPSNPPRKPEVHTGGKRFVKKDSTETQTLGGAE
FDLLASDGTAVKWTDALIKANTNKNYIAGEAVTGQPIKLKSHTDGTFEIK
GLAYAVDANAEGTAVTYKLKETKAPEGYVIPDKEIEFTVSQTSYNTKPTD
ITVDSADATPDTIKNNKRPSIPNTGGIGTAIFVAIGAAVMAFAVKGMKRR
TKDN

GBS80 contains a N-terminal leader or signal sequence region which is indicated by the underlined sequence above. One or more amino acids from the leader or signal sequence region of GBS80 can be removed. An example of such a GBS80 fragment is set forth below as SEQ ID NO: 8:

AEVSQERPAKTTVNIYKLQADSYKSEITSNGGIENKDGEVISNYAKLGDN
VKGLQGVQFKRYKVKTDISVDELKKLTTVEAADAKVGTILEEGVSLPQKT
NAQGLVVDALDSKSNVRYLYVEDLKNSPSNITKAYAVPFVLELPVANSTG
TGFLSEINIYPKNVVTDEPKTDKDVKKLGQDDAGYTIGEEFKWFLKSTIP
ANLGDYEKFEITDKFADGLTYKSVGKIKIGSKTLNRDEHYTIDEPTVDNQ
NTLKITFKPEKFKEIAELLKGMTLVKNQDALDKATANTDDAAFLEIPVAS
TINEKAVLGKAIENTFELQYDHTPDKADNPKPSNPPRKPEVHTGGKRFVK
KDSTETQTLGGAEFDLLASDGTAVKWTDALIKANTNKNYIAGEAVTGQPI
KLKSHTDGTFEIKGLAYAVDANAEGTAVTYKLKETKAPEGYVIPDKEIEF
TVSQTSYNTKPTDITVDSADATPDTIKNNKRPSIPNTGGIGTAIFVAIGA
AVMAFAVKGMKRRTKDN

GBS80 contains a C-terminal transmembrane region which is indicated by the underlined sequence near the end of SEQ ID NO: 7 above. One or more amino acids from the transmembrane region and/or a cytoplasmic region may be removed. An example of such a fragment is set forth below as SEQ ID NO:9:

MKLSKKLLFSAAVLTMVAGSTVEPVAQFATGMSIVRAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKTLNRDEHYTIDEPTVDNQNTLKITFKPEKFK
EIAELLKGMTLVKNQDALDKATANTDDAAFLEIPVASTINEKAVLGKAIE
NTFELQYDHTPDKADNPKPSNPPRKPEVHTGGKRFVKKDSTETQTLGGAE
FDLLASDGTAVKWTDALIKANTNKNYIAGEAVTGQPIKLKSHTDGTFEIK
GLAYAVDANAEGTAVTYKLKETKAPEGYVIPDKEIEFTVSQTSYNTKPTD
ITVDSADATPDTIKNNKRPSIPNTG

GBS80 contains an amino acid motif indicative of a cell wall anchor, shown in italics in SEQ ID NO: 7 above. In some recombinant host cell systems, it may be preferable to remove this motif to facilitate secretion of a recombinant GBS80 protein from the host cell. Thus the transmembrane and/or cytoplasmic regions and the cell wall anchor motif may be removed from GBS80. An example of such a fragment is set forth below as SEQ ID NO: 10.

MKLSKKLLFSAAVLTMVAGSTVEPVAQFATGMSIVRAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKTLNRDEHYTIDEPTVDNQNTLKITFKPEKFK
EIAELLKGMTLVKNQDALDKATANTDDAAFLEIPVASTINEKAVLGKAIE
NTFELQYDHTPDKADNPKPSNPPRKPEVHTGGKRFVKKDSTETQTLGGAE
FDLLASDGTAVKWTDALIKANTNKNYIAGEAVTGQPIKLKSHTDGTFEIK
GLAYAVDANAEGTAVTYKLKETKAPEGYVIPDKEIEFTVSQTSYNTKPTD
ITVDSADATPDTIKNNKRPS

Alternatively, in some recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition. In one embodiment, the leader or signal sequence region, the transmembrane and cytoplasmic regions and the cell wall anchor motif are removed from the GBS80 sequence. An example of such a GBS80 fragment is set forth below as SEQ ID NO: 11:

AEVSQERPAKTTVNIYKLQADSYKSEITSNGGIENKDGEVISNYAKLGDN
VKGLQGVQFKRYKVKTDISVDELKKLTTVEAADAKVGTILEEGVSLPQKT
NAQGLVVDALDSKSNVRYLYVEDLKNSPSNITKAYAVPFVLELPVANSTG
TGFLSEINIYPKNVVTDEPKTDKDVKKLGQDDAGYTIGEEFKWFLKSTIP
ANLGDYEKFEITDKFADGLTYKSVGKIKIGSKTLNRDEHYTIDEPTVDNQ
NTLKITFKPEKFKEIAELLKGMTLVKNQDALDKATANTDDAAFLEIPVAS
TINEKAVLGKAIENTFELQYDHTPDKADNPKPSNPPRKPEVHTGGKRFVK
KDSTETQTLGGAEFDLLASDGTAVKWTDALIKANTNKNYIAGEAVTGQPI
KLKSHTDGTFEIKGLAYAVDANAEGTAVTYKLKETKAPEGYVIPDKEIEF
TVSQTSYNTKPTDITVDSADATPDTIKNNKRPS

A particularly immunogenic fragment of GBS80 is located towards the N-terminus of the protein, and is given herein as SEQ ID NO: 12:

AEVSQERPAKTTVNIYKLQADSYKSEITSNGGIENKDGEVISNYAKLGDN
VKGLQGVQFKRYKVKTDISVDELKKLTTVEAADAKVGTILEEGVSLPQKT
NAQGLVVDALDSKSNVRYLYVEDLKNSPSNITKAYAVPFVLELPVANSTG
TGFLSEINIYPKNVVTDEPKTDKDVKKLGQDDAGYTIGEEFKWFLKSTIP
ANLGDYEKFEITDKFADGLTYKSVGKIKIGSKTLNRDEHYTIDEPTVDNQ
NTLKITFKPEKFKEIAELLKG

### Spb1

The wild-type Spb1 sequence from serotype III strain COH1 is SEQ ID NO: 13 herein:

MKKKMIQSLLVASLAFGMAVSPVTPIAFAAETGTITVQDTQKGATYKAYK
VFDAEIDNANVSDSNKDGASYLIPQGKEAEYKASTDFNSLFTTTTNGGRT
YVTKKDTASANEIATWAKSISANTTPVSTVTESNNDGTEVINVSQYGYYY
VSSTVNNGAVIMVTSVTPNATIHEKNTDATWGDGGGKTVDQKTYSVGDTV
KYTITYKNAVNYHGTEKVYQYVIKDTMPSASVVDLNEGSYEVTITDGSGN
ITTLTQGSEKATGKYNLLEENNNFTITIPWAATNTPTGNTQNGANDDFFY
KGINTITVTYTGVLKSGAKPGSADLPENTNIATINPNTSNDDPGQKVTVR
DGQITIKKIDGSTKASLQGAIFVLKNATGQFLNFNDTNNVEWGTEANATE
YTTGADGIITITGLKEGTYYLVEKKAPLGYNLLDNSQKVILGDGATDTTN
SDNLLVNPTVENNKGTELPSTGGIGTTIFYIIGAILVIGAGIVLVARRRL
RS

Wild-type SpbI contains a N-terminal leader or signal sequence region which is indicated by the underlined sequence above (aa 1-29). One or more amino acids from the leader or signal sequence region of SpbI can be removed. An example of such a SpbI fragment is set forth below as SEQ ID NO: 14:

AETGTITVQDTQKGATYKAYKVFDAEIDNANVSDSNKDGASYLIPQGKEA
EYKASTDFNSLFTTTTNGGRTYVTKKDTASANEIATWAKSISANTTPVST
VTESNNDGTEVINVSQYGYYYVSSTVNNGAVIMVTSVTPNATIHEKNTDA
TWGDGGGKTVDQKTYSVGDTVKYTITYKNAVNYHGTEKVYQYVIKDTMPS
ASVVDLNEGSYEVTITDGSGNITTLTQGSEKATGKYNLLEENNNFTITIP
WAATNTPTGNTQNGANDDFFYKGINTITVTYTGVLKSGAKPGSADLPENT
NIATINPNTSNDDPGQKVTVRDGQITIKKIDGSTKASLQGAIFVLKNATG
QFLNFNDTNNVEWGTEANATEYTTGADGIITITGLKEGTYYLVEKKAPLG
YNLLDNSQKVILGDGATDTTNSDNLLVNPTVENNKGTELPSTGGIGTTIF
YIIGAILVIGAGIVLVARRRLRS

The wild-type SpbI sequence contains an amino acid motif indicative of a cell wall anchor (LPSTG). In some recombinant host cell systems, it may be preferable to remove this motif to facilitate secretion of a recombinant SpbI protein from the host cell. Thus the cell wall anchor motif and sequence C-terminal to this motif may be removed from SpbI. An example of such a fragment is set forth below as SEQ ID NO: 15:

MKKKMIQSLLVASLAFGMAVSPVTPIAFAAETGTITVQDTQKGATYKAYK
VFDAEIDNANVSDSNKDGASYLIPQGKEAEYKASTDFNSLFTTTTNGGRT
YVTKKDTASANEIATWAKSISANTTPVSTVTESNNDGTEVINVSQYGYYY
VSSTVNNGAVIMVTSVTPNATIHEKNTDATWGDGGGKTVDQKTYSVGDTV
KYTITYKNAVNYHGTEKVYQYVIKDTMPSASVVDLNEGSYEVTITDGSGN
ITTLTQGSEKATGKYNLLEENNNFTITIPWAATNTPTGNTQNGANDDFFY
KGINTITVTYTGVLKSGAKPGSADLPENTNIATINPNTSNDDPGQKVTVR
DGQITIKKIDGSTKASLQGAIFVLKNATGQFLNFNDTNNVEWGTEANATE
YTTGADGIITITGLKEGTYYLVEKKAPLGYNLLDNSQKVILGDGATDTTN
SDNLLVNPTVENNKGTE

Alternatively, in some recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition. In one embodiment, the leader or signal sequence region, the cell wall anchor motif and sequence C-terminal to this motif are removed from SpbI. An example of such a SpbI fragment is set forth below as SEQ ID NO: 16:

AETGTITVQDTQKGATYKAYKVFDAEIDNANVSDSNKDGASYLIPQGKEA
EYKASTDFNSLFTTTTNGGRTYVTKKDTASANEIATWAKSISANTTPVST
VTESNNDGTEVINVSQYGYYYVSSTVNNGAVIMVTSVTPNATIHEKNTDA
TWGDGGGKTVDQKTYSVGDTVKYTITYKNAVNYHGTEKVYQYVIKDTMPS
ASVVDLNEGSYEVTITDGSGNITTLTQGSEKATGKYNLLEENNNFTITIP
WAATNTPTGNTQNGANDDFFYKGINTITVTYTGVLKSGAKPGSADLPENT
NIATINPNTSNDDPGQKVTVRDGQITIKKIDGSTKASLQGAIFVLKNATG
QFLNFNDTNNVEWGTEANATEYTTGADGIITITGLKEGTYYLVEKKAPLG
YNLLDNSQKVILGDGATDTTNSDNLLVNPTVENNKGTE

An E box containing a conserved glutamic residue has also been identified in SpbI (underlined), with a conserved glutamic acid at residue 423 (bold). The E box motif may be important for the formation of oligomeric pilus-like structures, and so useful fragments of SpbI may include the conserved glutamic acid residue.

The wild-type Spb1 sequence includes an internal methionine codon (Met-162) that has an upstream 12-mer TAATGGAGCTGT sequence that includes the core sequence (underlined) of a Shine-Dalgarno sequence. This Shine-Dalgarno sequence has been found to initiate translation of a truncated Spb1 sequence. To prevent translation initiation at this site the Shine-Dalgarno sequence can be disrupted in a Spb1-coding sequence used for expression. Although any suitable nucleotide can be mutated to prevent ribosome binding, the sequence includes a GGA glycine codon that is both part of the Shine-Dalgarno core and in-frame with the internal methionine codon. The third base in this codon can be mutated to C, G or T without changing the encoded glycine, thereby avoiding any change in Spb1 sequence.

### GBS59(6xD3)

The amino acid sequences of a number of suitable GBS59(6xD3) fusion proteins are provided below:

(Fusion E)

SEQ ID NO: 17

MGNNPTIENEPKEGIPVDKKITVNKTWAVDGNEVNKADETVDAVFTLQVK
DGDKWVNVDSAKATAATSFKHTFENLDNAKTYRVIERVSGYAPEYVSFVN
GVVTIKNNKDSNEPTPIGSGSGNKPGKKVKEIPVTPSNGEITVSKTWDKG
SDLENANVVYTLKDGGTAVASVSLTKTTPNGEINLGNGIKFTVTGAFAGK
FSGLTDSKTYMISERIAGYGNTITTGAGSAAITNTPDSDNPTPLGSGSGN
NPTEESEPQEGTPANQEIKVIKDWAVDGTITDANVAVKAIFTLQEKQTDG
TWVNVASHEATKPSRFEHTFTGLDNAKTYRVVERVSGYTPEYVSFKNGVV
TIKNNKNSNDPTPIGSGSGNKPGKDLTELPVTPSKGEVTVAKTWSDGIAP
DGVNVVYTLKDKDKTVASVSLTKTSKGTIDLGNGIKFEVSGNFSGKFTGL
ENKSYMISERVSGYGSAINLENGKVTITNTKDSDNPTPLGSGSGNKPGTD
LSEQPVTPEDGEVKVTKTWAAGANKADAKVVYTLKNATKQVVASVALTAA
DTKGTINLGKGMTFEITGAFSGTFKGLQNKAYTVSERVAGYTNAINVTGN
AVAITNTPDSDNPTPLGSGSGNNPTTENEPQTGNPVNKEITVRKTWAVDG
NEVNKGDEKVDAVFTLQVKDSDKWVNVDSATATAATDFKYTFKNLDNAKT
YRVVERVSGYAPAYVSFVGGVVTIKNNKNSNDPTPI
(Fusion F)

SEQ ID NO: 18

MGNNPTIENEPKEGIPVDKKITVNKTWAVDGNEVNKADETVDAVFTLQVK
DGDKWVNVDSAKATAATSFKHTFENLDNAKTYRVIERVSGYAPEYVSFVN
GVVTIKNNKDSNEPTPINPSEPKVVTYGRKFVKTNKDGKERLAGATFLVK
KDGKYLARKSGVATDAEKAAVDSTKSALDAAVKAYNDLTKEKQEGQDGKS
ALATVSEKQKAYNDAFVKANYSYEGSGSGNNPTEESEPQEGTPANQEIKV
IKDWAVDGTITDANVAVKAIFTLQEKQTDGTWVNVASHEATKPSRFEHTF
TGLDNAKTYRVVERVSGYTPEYVSFKNGVVTIKNNKNSNDPTPINPSEPK
VVTYGRKFVKTNQANTERLAGATFLVKKEGKYLARKAGAATAEAKAAVKT
AKLALDEAVKAYNDLTKEKQEGQEGKTALATVDQKQKAYNDAFVKANYSY
EGSGSGNKPGTDLSEQPVTPEDGEVKVTKTWAAGANKADAKVVYTLKNAT
KQVVASVALTAADTKGTINLGKGMTFEITGAFSGTFKGLQNKAYTVSERV
AGYTNAINVTGNAVAITNTPDSDNPTPLNPTQPKVETHGKKFVKVGDADA
RLAGAQFVVKNSAGKFLALKEDAAVSGAQTELATAKTDLDNAIKAYNGLT
KAQQEGADGTSAKELINTKQSAYDAAFIKARTAYTGSGSGNKPGKDLTEL
PVTPSKGEVTVAKTWSDGIAPDGVNVVYTLKDKDKTVASVSLTKTSKGTI
DLGNGIKFEVSGNFSGKFTGLENKSYMISERVSGYGSAINLENGKVTITN
TKDSDNPTPLNPTEPKVETHGKKFVKTNEQGDRLAGAQFVVKNSAGKYLA
LKADQSEGQKTLAAKKIALDEAIAAYNKLSATDQKGEKGITAKELIKTKQ
ADYDAAFIEARTAYEGSGSGNKPGKKVKEIPVTPSNGEITVSKTWDKGSD
LENANVVYTLKDGGTAVASVSLTKTTPNGEINLGNGIKFTVTGAFAGKFS
GLTDSKTYMISERIAGYGNTITTGAGSAAITNTPDSDNPTPLNPTEPKVV
THGKKFVKTSSTETERLQGAQFVVKDSAGKYLALKSSATISAQTTAYTNA
KTALDAKIAAYNKLSADDQKGTKGETAKAEIKTAQDAYNAAFIVARTAYE
GSGSGNNPTTENEPQTGNPVNKEITVRKTWAVDGNEVNKGDEKVDAVFTL
QVKDSDKWVNVDSATATAATDFKYTFKNLDNAKTYRVVERVSGYAPAYVS
FVGGVVTIKNNKNSNDPTPINPSEPKVVTYGRKFVKTNQDGSERLAGATF
LVKNSQSQYLARKSGVATNEAHKAVTDAKVQLDEAVKAYNKLTKEQQESQ
DGKAALNLIDEKQTAYNEAFAKANYSYE
(Fusion G)

SEQ ID NO: 19

MGNKPGKDLTELPVTPSKGEVTVAKTWSDGIAPDGVNVVYTLKDKDKTVA
SVSLTKTSKGTIDLGNGIKFEVSGNFSGKFTGLENKSYMISERVSGYGSA
INLENGKVTITNTKDSDNPTPLNPTEPKVETHGKKFVKTNEQGDRLAGAQ
FVVKNSAGKYLALKADQSEGQKTLAAKKIALDEAIAAYNKLSATDQKGEK
GITAKELIKTKQADYDAAFIEARTAYEGSGSGNNPTIENEPKEGIPVDKK
ITVNKTWAVDGNEVNKADETVDAVFTLQVKDGDKWVNVDSAKATAATSFK
HTFENLDNAKTYRVIERVSGYAPEYVSFVNGVVTIKNNKDSNEPTPINPS
EPKVVTYGRKFVKTNKDGKERLAGATFLVKKDGKYLARKSGVATDAEKAA
VDSTKSALDAAVKAYNDLTKEKQEGQDGKSALATVSEKQKAYNDAFVKAN
YSYEGSGSGNKPGKKVKEIPVTPSNGEITVSKTWDKGSDLENANVVYTLK
DGGTAVASVSLTKTTPNGEINLGNGIKFTVTGAFAGKFSGLTDSKTYMIS
ERIAGYGNTITTGAGSAAITNTPDSDNPTPLNPTEPKVVTHGKKFVKTSS
TETERLQGAQFVVKDSAGKYLALKSSATISAQTTAYTNAKTALDAKIAAY
NKLSADDQKGTKGETAKAEIKTAQDAYNAAFIVARTAYEGSGSGNNPTEE
SEPQEGTPANQEIKVIKDWAVDGTITDANVAVKAIFTLQEKQTDGTWVNV
ASHEATKPSRFEHTFTGLDNAKTYRVVERVSGYTPEYVSFKNGVVTIKNN
KNSNDPTPINPSEPKVVTYGRKFVKTNQANTERLAGATFLVKKEGKYLAR
KAGAATAEAKAAVKTAKLALDEAVKAYNDLTKEKQEGQEGKTALATVDQK
QKAYNDAFVKANYSYE
(Fusion H)

SEQ ID NO: 20

MGNNPTEESEPQEGTPANQEIKVIKDWAVDGTITDANVAVKAIFTLQEKQ
TDGTWVNVASHEATKPSRFEHTFTGLDNAKTYRVVERVSGYTPEYVSFKN
GVVTIKNNKNSNDPTPINPSEPKVVTYGRKFVKTNQANTERLAGATFLVK
KEGKYLARKAGAATAEAKAAVKTAKLALDEAVKAYNDLTKEKQEGQEGKT
ALATVDQKQKAYNDAFVKANYSYEGSGSGNKPGKKVKEIPVTPSNGEITV
SKTWDKGSDLENANVVYTLKDGGTAVASVSLTKTTPNGEINLGNGIKFTV
TGAFAGKFSGLTDSKTYMISERIAGYGNTITTGAGSAAITNTPDSDNPTP
LNPTEPKVVTHGKKFVKTSSTETERLQGAQFVVKDSAGKYLALKSSATIS
AQTTAYTNAKTALDAKIAAYNKLSADDQKGTKGETAKAEIKTAQDAYNAA
FIVARTAYEGSGSGNKPGKDLTELPVTPSKGEVTVAKTWSDGIAPDGVNV
VYTLKDKDKTVASVSLTKTSKGTIDLGNGIKFEVSGNFSGKFTGLENKSY
MISERVSGYGSAINLENGKVTITNTKDSDNPTPLNPTEPKVETHGKKFVK
TNEQGDRLAGAQFVVKNSAGKYLALKADQSEGQKTLAAKKIALDEAIAAY
NKLSATDQKGEKGITAKELIKTKQADYDAAFIEARTAYEGSGSGNNPTIE
NEPKEGIPVDKKITVNKTWAVDGNEVNKADETVDAVFTLQVKDGDKWVNV
DSAKATAATSFKHTFENLDNAKTYRVIERVSGYAPEYVSFVNGVVTIKNN
KDSNEPTPINPSEPKVVTYGRKFVKTNKDGKERLAGATFLVKKDGKYLAR
KSGVATDAEKAAVDSTKSALDAAVKAYNDLTKEKQEGQDGKSALATVSEK
QKAYNDAFVKANYSYE
(Fusion I)

SEQ ID NO: 21

MDGSILADSKAVPVKITLPLVNDNGVVKDAHVYPKNTETKPQVDKNFADK
ELDYANNKKDKGTVSASVGDVKKYHVGTKILKGSDYKKLIWTDSMTKGLT
FNNDIAVTLDGATLDATNYKLVADDQGFRLVLTDKGLEAVAKAAKTKDVE
IKITYSATLNGSAVVEVLETNDVKLDYGNNPTIENEPKEGIPVDKKITVN
KTWAVDGNEVNKADETVDAVFTLQVKDGDKWVNVDSAKATAATSFKHTFE
NLDNAKTYRVIERVSGYAPEYVSFVNGVVTIKNNKDSNEPTPINPSEPKV
VTYGRKFVKTNKDGKERLAGATFLVKKDGKYLARKSGVATDAEKAAVDST
KSALDAAVKAYNDLTKEKQEGQDGKSALATVSEKQKAYNDAFVKANYSYE
GSGSNGSLLAASKAVPVNITLPLVNEDGVVADAHVYPKNTEEKPEIDKNF
AKTNDLTALTDVNRLLTAGANYGNYARDKATATAEIGKVVPYEVKTKIHK
GSKYENLVWTDIMSNGLTMGSTVSLKASGTTETFAKDTDYELSIDARGFT
LKFTADGLGKLEKAAKTADIEFTLTYSATVNGQAIIDNPESNDIKLSYGN
KPGKDLTELPVTPSKGEVTVAKTWSDGIAPDGVNVVYTLKDKDKTVASVS
LTKTSKGTIDLGNGIKFEVSGNFSGKFTGLENKSYMISERVSGYGSAINL
ENGKVTITNTKDSDNPTPLNPTEPKVETHGKKFVKTNEQGDRLAGAQFVV
KNSAGKYLALKADQSEGQKTLAAKKIALDEAIAAYNKLSATDQKGEKGIT
AKELIKTKQADYDAAFIEARTAYEGSGSNGSILADSKAVPVKITLPLVNN
QGVVKDAHIYPKNTETKPQVDKNFADKDLDYTDNRKDKGVVSATVGDKKE
YIVGTKILKGSDYKKLVWTDSMTKGLTFNNNVKVTLDGEDFPVLNYKLVT
DDQGFRLALNATGLAAVAAAAKDKDVEIKITYSATVNGSTTVEIPETNDV
KLDYGNNPTEESEPQEGTPANQEIKVIKDWAVDGTITDANVAVKAIFTLQ
EKQTDGTWVNVASHEATKPSRFEHTFTGLDNAKTYRVVERVSGYTPEYVS
FKNGVVTIKNNKNSNDPTPINPSEPKVVTYGRKFVKTNQANTERLAGATF
LVKKEGKYLARKAGAATAEAKAAVKTAKLALDEAVKAYNDLTKEKQEGQE
GKTALATVDQKQKAYNDAFVKANYSYE

Further suitable sequences are provided in WO2011/121576.

GBS59(6xD3)-1523
The GBS59(6xD3)-1523 sequence is SEQ ID NO: 22
herein:
MGNNPTIENEPKEGIPVDKKITVNKTWAVDGNEVNKADETVDAVFTLQVK
DGDKWVNVDSAKATAATSFKHTFENLDNAKTYRVIERVSGYAPEYVSFVN
GVVTIKNNKDSNEPTPIGSGSGNKPGKKVKEIPVTPSNGEITVSKTWDKG
SDLENANVVYTLKDGGTAVASVSLTKTTPNGEINLGNGIKFTVTGAFAGK
FSGLTDSKTYMISERIAGYGNTITTGAGSAAITNTPDSDNPTPLGSGSGN
NPTEESEPQEGTPANQEIKVIKDWAVDGTITDANVAVKAIFTLQEKQTDG
TWVNVASHEATKPSRFEHTFTGLDNAKTYRVVERVSGYTPEYVSFKNGVV
TIKNNKNSNDPTPIGSGSGNKPGKDLTELPVTPSKGEVTVAKTWSDGIAP
DGVNVVYTLKDKDKTVASVSLTKTSKGTIDLGNGIKFEVSGNFSGKFTGL
ENKSYMISERVSGYGSAINLENGKVTITNTKDSDNPTPLGSGSGNKPGTD
LSEQPVTPEDGEVKVTKTWAAGANKADAKVVYTLKNATKQVVASVALTAA
DTKGTINLGKGMTFEITGAFSGTFKGLQNKAYTVSERVAGYTNAINVTGN
AVAITNTPDSDNPTPLGSGSGNNPTTENEPQTGNPVNKEITVRKTWAVDG
NEVNKGDEKVDAVFTLQVKDSDKWVNVDSATATAATDFKYTFKNLDNAKT
YRVVERVSGYAPAYVSFVGGVVTIKNNKNSNDPTPIGSGGGGETGTITVQ
DTQKGATYKAYKVFDAEIDNANVSDSNKDGASYLIPQGKEAEYKASTDFN
SLFTTTTNGGRTYVTKKDTASANEIATWAKSISANTTPVSTVTESNNDGT
EVINVSQYGYYYVSSTVNNGAVIMVTSVTPNATIHEKNTDATWGDGGGKT
VDQKTYSVGDTVKYTITYKNAVNYHGTEKVYQYVIKDTMPSASVVDLNEG
SYEVTITDGSGNITTLTQGSEKATGKYNLLEENNNFTITIPWAATNTPTG
NTQNGANDDFFYKGINTITVTYTGVLKSGAKPGSADLPENTNIATINPNT
SNDDPGQKVTVRDGQITIKKIDGSTKASLQGAIFVLKNATGQFLNFNDTN
NVEWGTEANATEYTTGADGIITITGLKEGTYYLVEKKAPLGYNLLDNSQK
VILGDGATDTTNSDNLLVNPTVENNKGTE

Further suitable sequences are provided in EP14179945.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

In some implementations, the term “comprising” refers to the inclusion of the indicated active agent, such as recited polypeptides, as well as inclusion of other active agents, and pharmaceutically acceptable carriers, excipients, emollients, stabilizers, etc., as are known in the pharmaceutical industry. In some implementations, the term “consisting essentially of” refers to a composition, whose only active ingredient is the indicated active ingredient(s), however, other compounds may be included which are for stabilizing, preserving, etc. the formulation, but are not involved directly in the therapeutic effect of the indicated active ingredient. Use of the transitional phrase “consisting essentially” means that the scope of a claim is to be interpreted to encompass the specified materials or steps recited in the claim, and those that do not materially affect the basic and novel characteristic(s) of the claimed invention. See, In re Herz, 537 F.2d 549, 551-52, 190 USPQ 461, 463 (CCPA 1976) (emphasis in the original); see also MPEP § 2111.03. Thus, the term “consisting essentially of” when used in a claim of this invention is not intended to be interpreted to be equivalent to “comprising”. The term “consisting of” and variations thereof includes including and limited to unless expressly specified otherwise. The term “about” in relation to a numerical value x means, for example, x+10%, x+5%, x+4%, x+3%, x+2%, x+1%, The word “substantially” does not exclude “completely” e.g. a composition which is “substantially free” from Y may be completely free from Y. Where necessary, the word “substantially” may be omitted from the definition of the invention.

It will be appreciated that sugar rings can exist in open and closed form and that, whilst closed forms are shown in structural formulae herein, open forms are also encompassed by the invention. Similarly, it will be appreciated that sugars can exist in pyranose and furanose forms and that, whilst pyranose forms are shown in structural formulae herein, furanose forms are also encompassed. Different anomeric forms of sugars are also encompassed.

Unless specifically stated, a process comprising a step of mixing two or more components does not require any specific order of mixing. Thus components can be mixed in any order. Where there are three components then two components can be combined with each other, and then the combination may be combined with the third component, etc.

Antibodies will generally be specific for their target. Thus they will have a higher affinity for the target than for an irrelevant control protein, such as bovine serum albumin.

Unless otherwise stated, identity between polypeptide sequences is preferably determined by the Smith-Waterman homology search algorithm as implemented in the MPSRCH program (Oxford Molecular), using an affine gap search with parameters gap open penalty=12 and gap extension penalty=1.

### MODES FOR CARRYING OUT THE INVENTION

### Bacterial Strains and Growth Conditions.

GBS type IX strain IT-NI-016 (isolated from a neonatal Early Onset Disease case) was kindly provided by Alberto Berardi (Policlinico di Modena, Italy), isolated and typed by Latex and molecular approaches in the frame of the DEVANI study (144). Capsular genotype of type IX isolates was confirmed by genome analysis (see below). Strain 2603 V/R (serotype V) was obtained from Dennis Kasper (Harvard Medical School, Boston, Mass.). Strain CJB111 (serotype V) was obtained from Carol Baker (Baylor College of Medicine, Houston, Tex.). GBS wild type strains were grown at 37° C. in Todd Hewitt broth (Difco Laboratories) or in trypticase soy agar supplemented with 5% sheep blood. Transformed clones bearing plasmid “pAM-IX” and “pAM-IX-V” (see below) were selected and propagated in the aforementioned media, with the addition of chloramphenicol (Chl) (10 μg/ml). For plasmid cloning E. coli HB101 Competent Cells (Promega) were used. Cells were grown at 37° C. in an orbital shaking incubator (180 rpm) in Luria-Bertani (LB, Difco laboratories) medium or on 15 g/L agar plates (LBA). Chl was used for selection of positive clones (20 μg/ml).

### Genetic Engineering of GBS Types IX, V and VII

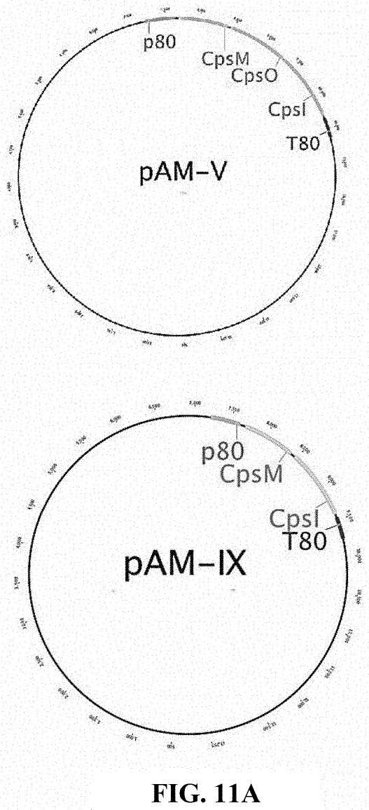

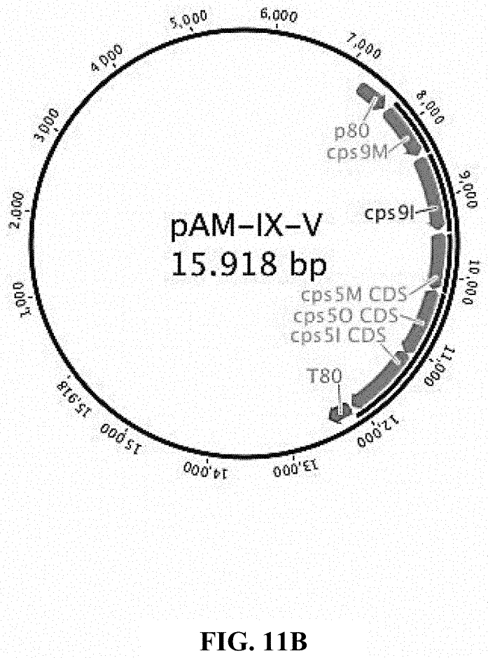

A plasmid was designed to obtain the chimeric-CPS expressing strain. A DNA fragment, consisting of the cps operon type IX-specific genes (cps9M and cps9I) was amplified by PCR from S. agalactiae IT-NI-016 genomic DNA using specifically designed primers (SEQ ID NO: 23: pAM-IX-F: ‘GCGGCGCGGCCGCGACATATTTGCTCTGATATGGCAG’; and SEQ ID NO: 24: pAM-IX-R: ‘GCGGCAGATCTGGGATAATGATACTAATCATCTTC’) and the following reaction cycle: 1′ at 98° C.; 10″ at 98° C., 20″ at 55° C., 3′ at 72° C. (30 cycles); 7′ at 72° C. The resulting fragment (cps9M-9I insert, SEQ ID NO: 43) was digested with NotI/BglII and ligated into the expression vector pAM-p80 (145) (SEQ ID NO: 44) to obtain plasmid pAM-cps9MI, (cps9M and cps9I; “pAM-IX”, SEQ ID NO: 45—FIG. 11). The plasmid was purified from the HB101 selected clone, sequenced, and used to transform electrocompetent GBS 2603 V/R cells or CJB111 cells by electroporation at 1,800 V as previously described (146). This configuration results in a strain with a glycosyltransferase repertoire consisting of a single copy of the serotype V specific genes cps5MOI and multiple copies of the type-IX specific genes.

DNA fragments consisting of the cps type V-specific (cps5M, cps5O, and cps5I, SEQ ID NOs: 40, 41 and 42) were amplified by PCR from S. agalactiae CJB111 genomic DNA, using specifically designed primers:

SEQ ID NO: 25:
pAM-V-F
GCGGCGCGGCCGCGCTCTGATATGGCAGGAGGTAAGG 37
SEQ ID NO: 26:
pAM-V-R
GCGGCAGATCTGGGATAATGATACTAACTTTATCC 35)

and the following reaction cycle: 1 min at 98° C.; 10 s at 98° C., 20 s at 55° C., and 3 min at 72° C. (30 cycles); and 7 min at 72° C. The resulting fragment (cps5MOI insert, SEQ ID NO: 53) was cloned into the expression vector pAM-p80 (26) to obtain plasmid pAM-cps5MOI (containing cps5M, cps5O, and cps5I; “pAM-V” SEQ ID NO: 52—FIG. 11). Plasmid was purified and used to transform GBS by electroporation. Strain IT-NI-016 (serotype IX) was transformed with pAM-V. Both plasmids were also used to transform serotype VII strain CZ-PW-045.

We investigated the effect of varying the dosing of serotype-specific cps genes on the CPS structure by building a new vector where the cps5MOI region was placed downstream of the cps9MI region of pAM-IX. This resulted in a new strain expressing a more balanced chimeric-CPS V-IX, i.e. PS V-IXb. To achieve this, a DNA fragment, consisting of the cps operon type V-specific genes (cps5M, cps5O and cps5I) was amplified by PCR from S. agalactiae CJB111 genomic DNA using specifically designed primers:

pAM-V-IX-F (SEQ ID NO: 54):
‘GCGGCAGATCTGTAAGGAAGGAAAATGATACCTAAAGTTAT’;
and
pAM-V-R: (SEQ ID NO: 26):
‘GCGGCAGATCTGGGATAATGATACTAACTTTATCC’)

and the following reaction cycle: 1′ at 98° C.; 10″ at 98° C., 20″ at 55° C., 3′ at 72° C. (30 cycles); 7′ at 72° C. The resulting fragment was digested with BglII and ligated into the previously generated vector pAM-cps9MI to obtain plasmid pAM-cps9MI-cps5MOI, (cps9M, cps9I, cps5M, cps5O and cps5I insert: SEQ ID NO:56; “pAM-IX-V”: SEQ ID NO: 55). The obtained plasmid was purified from the HB101 selected clones, sequenced, and used to transform electrocompetent GBS 2603 V/R cells by electroporation at 1,800 V, as previously described (146).

### Sera and Monoclonal Antibody Reagents

Mouse monoclonal antibodies (mAbs) directed against CRM197-conjugated GBS PS IX and PS V were generated by Areta International using standard protocols. Briefly, B-cell hybridoma clones were isolated from spleen cells of immunized CD1 mice with the respective purified capsular polysaccharide conjugated to CRM197. Positive clones were first selected by ELISA and then culture supernatants were screened for binding to the surface of the matching reference strain by flow cytometry. Positive primary hybridoma clones were subjected to single cell cloning and sub-cloning by limiting dilution. Monoclonality of a clone was accepted only when all the wells of a microtitre plate with growing cells gave positive reaction in indirect ELISA after repeated sub-cloning. The selected mAbs were finally purified by protein G affinity chromatography. Classes and subclasses of the monoclonal antibodies were determined by IsoQuick Mouse Monoclonal Isotyping Kit (Sigma).

For immunochemical detection of chimeric polysaccharide, a fraction of the monoclonal antibodies was biotinylated using the EZ-Link Sulfo-NHS-LC-Biotinylation Kit (Thermo Scientific), according to the manufacturer instructions. Animal treatments were performed in compliance with the Italian laws and approved by the institutional review board (Animal Ethical Committee) of Novartis Vaccines and Diagnostics, Siena, Italy.

### CPS Serotyping and Flow Cytometry Analysis

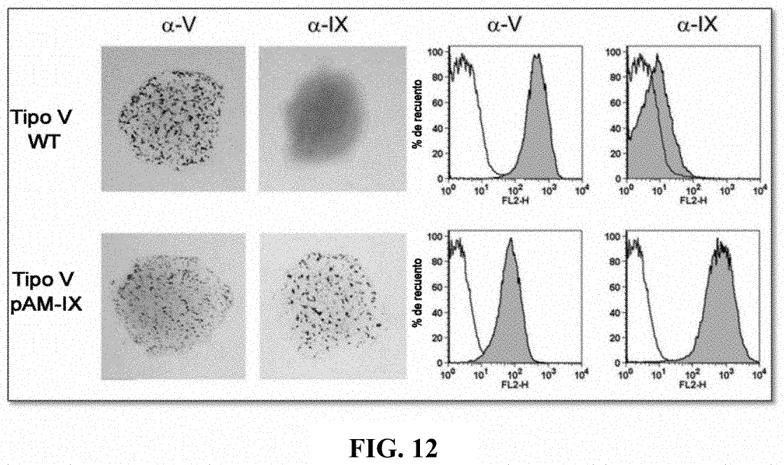

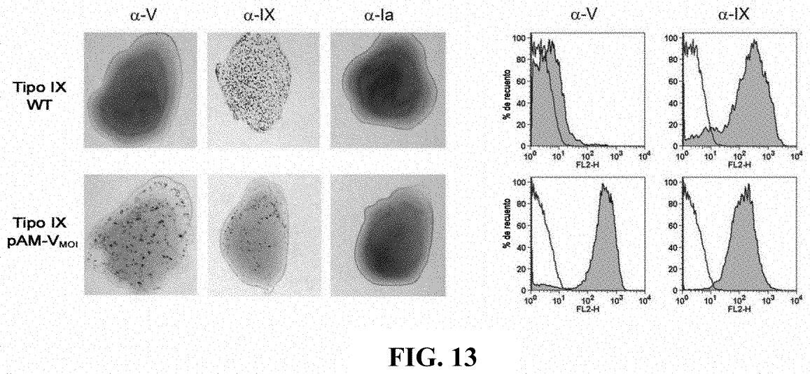

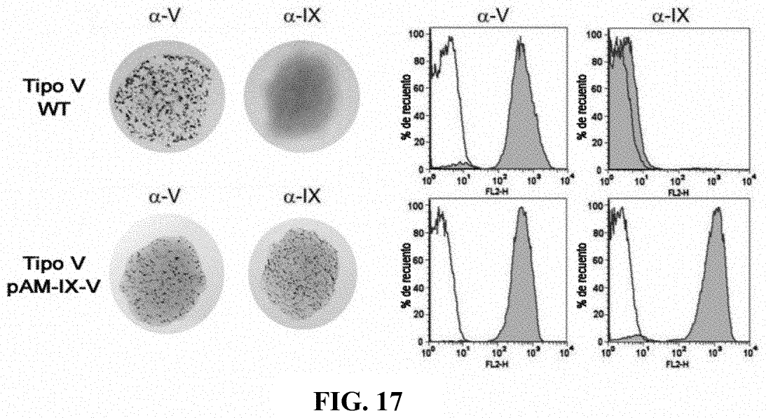

GBS serotyping by latex agglutination assay was conducted using the Strep-B-Latex kit (Statens Serum Institut) according to manufacturer's instructions. Flow cytometry using specific anti-capsular polysaccharide antibodies was performed. Bacteria grown in THB to exponential phase were harvested and fixed in PBS containing 0.1% (w/v) PFA for 1 h at 37° C. The fixed cells were washed with PBS and incubated for 1 h at room temperature with immune mouse sera raised against type V or type IX purified polysaccharides, diluted 1:200 in PBS containing 0.1% BSA. The cells were incubated for 1 hour at 23° C. with R-phycoerythrin-conjugated F(ab)2 goat anti-mouse immunoglobulin G, diluted 1:100 in PBS containing 0.1% BSA. All data were collected using a BD FACS Calibur and a BD FACS CANTO II (BD Bioscience) by acquiring 10,000 events, and data analysis was performed with Flow-Jo software (v.8.6, TreeStar Inc.). PS V-IX gives a positive signal indicating that the type V(pAM-IX) strain produces chimeric capsular polysaccharide chains that contain repeating units specifically recognized by both type V- and IX-specific mAbs (FIG. 12). Heterologous in-trans expression of cps9M and cps9I allows GBS 2603 (V) to assemble capsular polysaccharides reacting with both type V and IX CPS specific antisera (FIG. 12). Heterologous in-trans expression of cps5M, cps5O and cps5I allows GBS IT-NI-016 (IX) to assemble capsular polysaccharides reacting with both type IX and V CPS specific antisera (FIG. 13). Serological confirmation of the fact that the type V (pAM-IX-V) produces chimeric capsular polysaccharide chains that contain repeating units specifically recognised by both type V- and type IX-specific mAbs has been confirmed (FIG. 17).

### DNA Sequence Analysis of Cps Capsule Biosynthetic Clusters

For alignment comparisons, nucleotide sequences of the cps serotype-specific region from the type V reference strain were retrieved from the NCBI database (2603V/R, accession number NC 004116). The cps serotype-specific region from the type IX isolate (IT-NI-016) was extracted from the genomic sequence (147). Multiple and pairwise sequence alignments were performed with MUSCLE using Geneious version 7.05 (Biomatters, http://www.geneious.com/).

### Isolation and Purification of the Chimeric Type V-IX Capsular Polysaccharide

The GBS engineered strain 2603 V/R (pAM-IX) was used for preparation of the chimeric-CPS V-IX from 8 liters of bacterial culture grown to stationary phase in THB with chloramphenicol (10 μg/ml). In order to purify the polysaccharide, the bacterial pellet was recovered by centrifugation at 4,000 rpm for 30 min, washed in PBS and incubated with 0.8 N NaOH at 37° C. for 36 hours. After centrifugation at 4,000 rpm for 30 min, 1 M TRIS buffer (1:9 v/v) was added to the supernatant and diluted with 3 N HCl to reach a neutral pH. To further purify the chimeric type V-IX CPS, 2 M CaCl2 (0.1 M as final concentration) and ethanol (30% v/v as final concentration) were added to the solution. After centrifugation at 4,000 rpm for 30 min, the supernatant was subjected to a tangential flow filtration on a 10 kDa MW cut-off (Hydrosart Sartorius, 0.2 m2 surface) against 16 volumes of 50 mM TRIS/500 mM NaCl pH 8.8, and 10 volumes of 10 mM sodium phosphate pH 7.2.

The sample was then concentrated using a rotary evaporator (Rotavapor®, Büchi) and divided into 3 ml aliquots that were separately purified by Size Exclusion Chromatography (SEC) using a Sephacryl® S-500 resin packed column, pre-equilibrated in 100 mM NaPO4/1 M NaCl pH 7.2. The chromatographic separation was performed on an AKTA pure system (GE Healthcare) at 0.3 ml/min flow in 10 mM NaPO4/150 mM NaCl pH 7.2. The polysaccharide was detected by measuring UV absorption at 214 nm, 254 nm, and 280 nm and collected in fractions in the first eluted peak, appearing mainly as a single large peak. The polysaccharide solution was subjected to a desalting step through a Sephadex® G-15 (GE Healthcare) resin packed column, in water at 1 ml/min flow. To reconstitute full N-acetylation of possibly present GlcpNAc and NeupNAc residues, a 1:1 diluted solution of 4.15 μL/mL acetic anhydride in ethanol was added to the sample, and the reaction was incubated at room temperature for 2 hrs. The sample was concentrated using a Rotavapor and injected into a Sephadex® G-15 packed column to purify the re-acetylated polysaccharide. The purity of the polysaccharide preparation was assessed by colorimetric assays, which indicated a content of residual proteins and nucleic acids below 1% w/w. Highly purified GBS type V, IX and V-IX CPS were obtained by applying a similar purification process as described above for the chimeric PS V-IX.

### Immunochemical Detection of Chimeric Polysaccharide

### Sandwich ELISA

Microtiter wells were coated overnight at 4° C. with 100 μl of 2.5 μg/ml of each coating mAB in PBS, according to the experimental scheme. To block additional protein-binding sites, the wells were treated for 2 h at RT with 350 μl of 3% BSA in PBS. The plates were then incubated for 2 h at 37° C. with decreasing amounts of polysaccharide according to the experimental design. The specific biotinylated labeling monoclonal antibody diluted 1:100 in PBS/0.05% Tween(PBST)/1% BSA was added to the wells, followed by a 1 h incubation at 37° C. with shaking. After extensive washing, the plates were incubated for 45 min with 100 μl of horseradish peroxidase-conjugated streptavidin (Thermo Scientific) diluted 1:200 in PBST/1% BSA. After washing, a chromogenic substrate was added to the bound conjugated enzyme, and absorbance at 450 nm was determined.

### Sandwich Dot-Blot

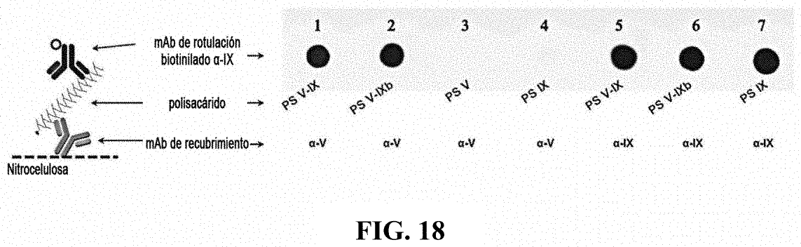

To reveal the chimeric nature of polysaccharide molecules a sandwich dot blot was set up in which polysaccharides were captured on a membrane, coated with a specific mAb against native PS V, and then revealed with a second mAb binding with high affinity to PS IX. Results (FIG. 18) showed that both the obtained chimeric polysaccharides (PS V-IX and PS V-IXb) were positively revealed by this approach, differently from the native PS V and PS IX, which represented negative controls. This data demonstrates that PS V-IX and PS V-IXb are chimeric capsular polysaccharides (cCPS), consisting of high molecular weight hybrid chains that inherit type V and IX characteristic epitopes since it is bound by both serotype-specific mAbs with high affinity and specificity.

Nitrocellulose membrane was spotted with 5 μl of each coating mAb, concentrated 0.45 mg/ml in PBS, according to the experimental scheme. To block additional protein-binding sites, the membrane was incubated o/n at 4° C. with PBST/5% blocking reagent (BioRad) with shaking. The membrane was cut to separate the original mAb spots. Each of the resulting nitrocellulose discs were then separated in one of the 12 wells of a cell culture plate (Costar). Each well was filled with 500 μl of the specific polysaccharide diluted to 25 μg/ml in PBST/3% blocking reagent, following the experimental design (FIG. 18). The plate was incubated for 2 h at RT with mild shaking. The specific biotinylated labeling anti-PS-IX monoclonal antibody, diluted 1:100 in 500 μl PBS T/3% blocking reagent, was added to each well followed by a 1 h incubation at RT with shaking. After extensive washing, the plate was incubated for 45 min with 500 μl of horseradish peroxidase-conjugated streptavidin (Thermo Scientific) diluted 1:5000 in PBST/3% blocking reagent. The blot was developed using the SuperSignal® West Pico Chemiluminescent Substrate (Thermo Scientific) following manufacturer instructions.

### Competitive ELISA

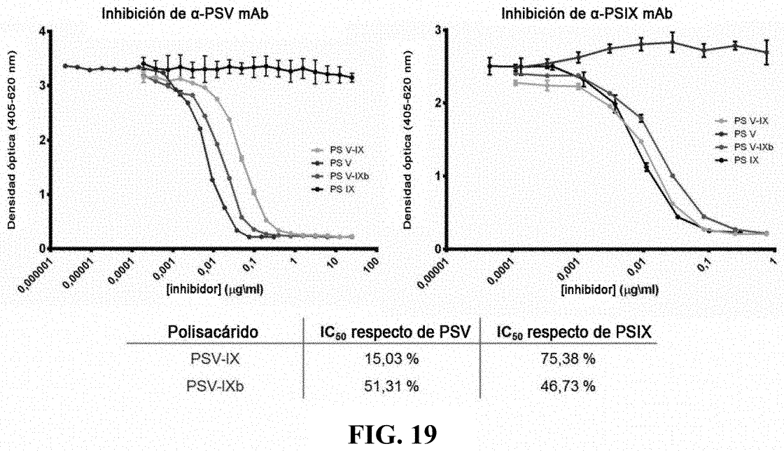

To measure the binding efficiency of the CPS-specific mAbs to the cCPS, and obtain a more quantitative estimate of their structural features, a competitive ELISA was set up. Results (FIG. 19) confirmed that PS V-IXb binds to both anti-type-V-specific mAbs and anti-type-IX-specific mAbs with half the efficiency of the native polysaccharides at the same concentration. Conversely, PS V-IX is able to bind anti-type-V-specific mAbs at the 15% and anti-type-IX-specific mAbs at the 75% respect to the native polysaccharides. These findings confirm that the balance in serotype-specific gene copy number leads to a balanced epitope ratio in the final cCPS.

Coating with GBS PS V or IX Conjugated to HAS-Adh
1 μg/ml in PBS pH 7.4 (100 μl/well) for V and IX in PBS pH 7.4 (100 μl/well)
Incubation at 4° C. O\N
Washing 3× washing buffer (0.05% Tween 20 in 1×PBS)

### Post-Coating

Dispense 250 μl/well of (2% BSA, 0.05% Tween 20 in 1×PBS)
Incubation for 1.5 hours at 37° C.
Aspiration
   mAb Pre-Incubation with PS

### mAb α-CRM-V

PS dilution in a separated polypropylene microtiter plate (17 dilutions, dilution buffer);
   IX: 2-fold step (first dilution in plate: 25 μg/mL of PS),
   V-IX: 2-fold step (first dilution in plate: 25 μg/mL of PS),
   V-IXb: 2-fold step (first dilution in plate: 25 μg/mL of PS),
   V: 2-fold step (first dilution in plate: 0.3 μg/mL of PS),

### mAb α-CRM-IX

PS dilution in a separated polypropylene microtiter plate (9 dilutions, dilution buffer);
   IX: 3-fold step (first dilution in plate: 0.3 μg/mL of PS),
   V-IX: 3-fold step (first dilution in plate: 0.75 μg/mL of PS),
   V-IXb: 3-fold step (first dilution in plate: 0.75 μg/mL of PS),
   V: 3-fold step (first dilution in plate: 0.75 μg/mL of PS),
A fixed concentration of mAb was added in each well (equal volume of test PS): 0.03 μg/mL for 12F1/H8 (α-CRM-V), 0.1 μg/mL for 17B2/F6 (α-CRM-IX).
Incubation for 20 min at r.t.

### Competition for Binding to mAb

The pre-incubation mixture was transferred to the coated and saturated plate
Incubation for 1 hour at 37° C.
Washing 3× washing buffer (0.05% Tween 20 in 1×PBS)

### Secondary Antibody

100 μL of a solution of AP-Conjugated anti-mouse IgG 1:2000 in dilution buffer were dispensed in each well
Incubation for 1.5 hours at 37° C.
Washing 3× washing buffer (0.05% Tween 20 in 1×PBS)

### Substrate Addition

Addition of 100 μL of p-NitroPhenylPhosphate (p-NPP) 1.0 mg/mL in substrate buffer
Incubation for 30 minutes at room temperature
Addition of 100 μL of EDTA 7% to stop the enzymatic reaction.

Plate reading at 405-620 nm.

### Nuclear Magnetic Resonance Spectroscopy

1H NMR experiments were recorded on Bruker Avance III 400 MHz spectrometer, equipped with a high precision temperature controller, and using 5-mm broadband probe (Bruker). TopSpin version 2.6 software (Bruker) was used for data acquisition and processing.

Spectra were collected at 25 or 35+/−0.1° C. with 32 k data points over a 10 ppm spectral width, accumulating 128 scans. Spectra were weighted with 0.2 Hz line broadening and Fourier-transformed. The transmitter was set at the water frequency which was used as the reference signal (4.79 ppm).

All mono-dimensional proton NMR spectra were obtained in quantitative manner using a total recycle time to ensure a full recovery of each signal (5× Longitudinal Relaxation Time T1). To better analyze the differences between the two obtained cCPS (PS V-IX and PS V-IXb) we performed carbon NMR spectroscopy experiments on both the chimeric and native polysaccharides.

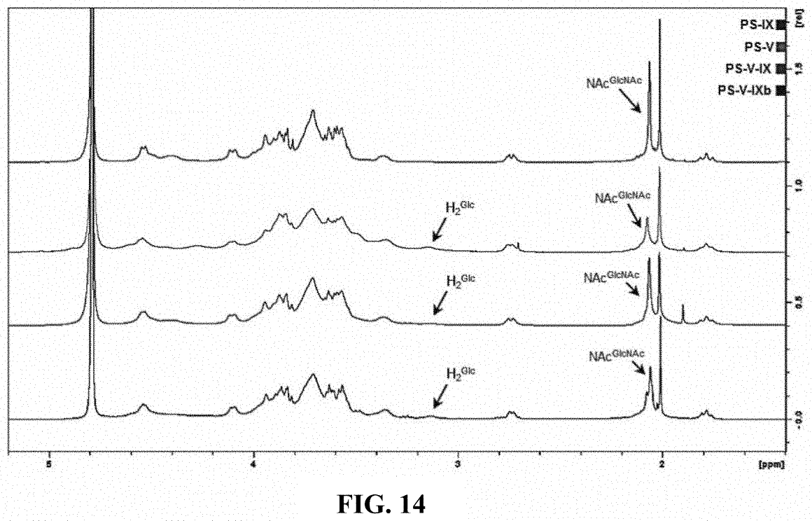

The PS V-IX 1H NMR spectrum is highly similar to that of PS V and PS IX and contains features that are characteristic of both capsular polysaccharides. The PS V-IXb ¹H NMR spectrum has a higher intensity of PS V-signature peaks respect to PSV-IX (FIG. 14). The average repeating unit composition of the two chimeric polysaccharides (PS V-IX and PS V-IXb) was estimated by DEPT NMR. In the region of the spectrum spanning approximately from 21.5 to 23 ppm resonate the CH3 carbons of the N-acetyl groups of GlcpNAc and NeupNAc. The chemical shift of the PS IX branch GlcpNAc CH3 is different from the corresponding one in the PS V spectrum (22.55 and 22.61 ppm, respectively). Furthermore, the signal at 21.8 ppm in PS IX DEPT spectrum was assigned to the backbone GlcpNAc CH3 and is therefore absent in PS V spectrum.

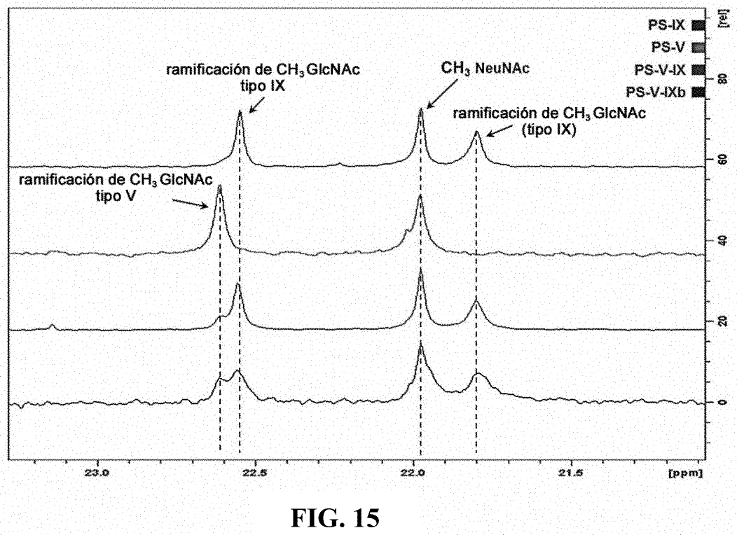

Starting from these observations it was possible to estimate the relative ratio of PS V to PS IX structural features for both cCPS, from the integration of the peak areas relative to these CH3 signals. As it is evident from the zoomed spectra superimposition (FIG. 15), type V and IX features ratio is approximately 1:3 in PS V-IX, are roughly one fourth the intensity of type IX, while the ratio is close to 1:1 in PS V-IXb. These observations suggest that the addition of a higher dose of cps5 genes presumably result in an increased activity of serotype-V-specific glycosyltransferases and, ultimately in a more balanced ratio of PS V to IX physicochemical features respect to PS V-IX. About 75% of the repeating units of the chimeric type V-IX polysaccharide are type IX while the remaining 25% are type V (FIG. 15). About 50% of the repeating units of the chimeric type V-IXb polysaccharide are type IX while the remaining 50% are type V (FIG. 15).

### Conjugate Production

Purified chimeric capsular polysaccharides from engineered Streptococcus agalactiae serotypes V and IX were conjugated to carrier protein by periodate oxidation followed by reductive amination following the procedures disclosed in ref 2 with some modifications. CRM197 was used as the carrier protein although the processes are applicable to other carrier proteins such as tetanus toxoid, GBS80, GBS59, GBS59(6xD3), etc.

### (1) Oxidation Reaction

An oxidation reaction was performed to generate aldehydic groups at the C8 of sialic acid residues by oxidative cleavage of the C8-C9 diol bond. The reaction was performed by adding 0.1M sodium periodate solution to the purified chimeric capsular polysaccharide solution and maintaining under stirring in the dark at least 2 hours.

The solution was immediately purified by ultrafiltration step to remove formaldehyde and Sodium periodate byproducts, such as iodate ions, generated during the reaction. Ultrafiltration was performed with a tangential flow diafiltration/concentration using 30 kD UF regenerated cellulose membranes (1 membrane Hydrosart 30 kD 0.1 sm) against 13 volumes of Sodium phosphate 100 mM pH 7.2 buffer. The 30 kD membrane retained polysaccharide and the conjugate was recovered in the UF retentate. The oxidised polysaccharide was 0.2 μm filtered and stored at 2°-8° C. for no more than 7 days.

### (2) Conjugation Reaction

The conjugation reaction occurs between some aldehydic groups generated by oxidation reaction and some ε-amino groups of lysines of the protein carrier, by reductive amination in presence of sodium cyanoborohydride. Periodate treated chimeric capsular polysaccharide was diluted with sodium phosphate 100 mM and CRM197 concentrate bulk was added to obtain a final concentration of 6.35 mg/mL as PS concentration. The target reaction conditions for the CPS-CRM conjugation are:
Polysaccharide/CRM ratio (0.75/1 w/w),
Saccharide Concentration (6.35 mg/mL)
NaCNBH3 (6.35 mg/mL).

The polysaccharide CRM ratio was used to guarantee almost complete conversion of the polysaccharide. The reaction was performed at room temperature (RT) for at least 10 hours, but no more than 28 hours, at pH 7.2 until a CRM conversion of at least 45% (monitored by in process SEC-HPLC test) was achieved.

### (3) Dilution of Crude Conjugate

At the end of conjugation reaction water for injection (WFI) was added to obtain a final buffer concentration of sodium phosphate 35 mM. The product was 0.2 μm filtered and stored at 2°-8° C. for no more than 24 hours, if not used immediately.

### (4) Hydroxyapatite Chromatography

Glycoconjugate was separated from unconjugated CRM by hydroxyapatite chromatography.

The glycoconjugate was collected in the flow through while CRM binds to the resin and is removed. The column was packed with Hydroxylapatite Type I resin and the purification conditions were:

1) Equilibration: 35 mM Sodium Phosphate pH=7.2 (5 Column Volumes)
2) Loading: 35 mM Sodium Phosphate pH=7.2 (2.9 Column Volumes)
3) Stripping: 400 mM Sodium Phosphate pH=6.8 (2 Column Volumes) The product was 0.2 μm filtered and stored at 2°-8° C. for no more than 24 hours.

### (5) Quenching Reaction

A quenching step was utilized to remove residual saccharide aldehydic groups by reaction with sodium borohydride (NaBH4). The quenching reaction was performed using a 10 mg/mL sodium borohydride solution at a molar excess of 25:1 with respect to the estimated oxidised sialic acid (that is 20% of total sialic acid). The reaction was performed for at least 2 hours maintaining the pH at 8.3±0.2 by 500 mM sodium phosphate addition. A final 30 kD Ultrafiltration was used to remove conjugation and quenching low molecular weight reagents and by-products and to concentrate it to a range of about 1.0 to 1.5 mg/mL as saccharide concentration.

### Immunisation and Challenge

### PS V-IX

A mouse challenge model of GBS infection was used to verify the protective efficacy of the antigens, as previously described (148). In brief, groups of eight to sixteen CD-1 female mice (age, 6-8 wk) were immunized with chimeric polysaccharide conjugate or buffer (PBS) formulated with Alum adjuvant. Protection values were calculated as [(% dead in control−% dead in vaccine)/% dead in control]×100.

| | # of | Treatment 1 | | Treatment 2 | | Adjuvant 1 | |
|---|---|---|---|---|---|---|---|
| Group | mice | ID | Quantity | ID | Quantity | ID | Quantity |
| 1 | 8 | CTRL | | | | Alum | 2 mg/ml |
| 2 | 16 | CRM-V | 0.25 μg | | | Alum | 2 mg/ml |
| 3 | 16 | CRM-IX | 0.441 μg | | | Alum | 2 mg/ml |
| 4 | 16 | CRM-V | 0.25 μg | CRM-IX | 0.441 μg | Alum | 2 mg/ml |
| 5 | 16 | CRM-V-IX | 0.691 μg | | | Alum | 2 mg/ml |
| 6 | 16 | CRM-IX | 0.25 μg | | | Alum | 2 mg/ml |
| 7 | 16 | CRM-V-IX | 0.25 μg | | | Alum | 2 mg/ml |

| | Challenge with Serotype IX | |
|---|---|---|
| Antigen | SURVIVAL # | SURVIVAL % |
| Control (Alum) | 23/70 | 33 |
| WT PS conjugate CRM-V | 77/150 | 51 |
| (0.25 μg PS) | | |
| WT PS conjugate CRM-IX | 133/138 | 96 |
| (0.441 μg PS) | | |
| Mix WT PS Conjugate: CRM-V + | 148/160 | 93 |
| CRM-IX (0.25 μg + 0.441 μg each PS) | | |
| Chimeric PS conjugate: CRM-V-IX | 116/128 | 91 |
| (0.691 μg PS) | | |
| WT PS conjugate: CRM-IX | 131/135 | 97 |
| (0.25 ug PS) | | |
| Chimeric PS conjugate: CRM-V-IX | 81/99 | 82 |
| (0.25 ug each PS) | | |

Vaccination of mice with conjugates comprising the type V-IX chimeric capsular polysaccharide protected mice against challenge with GBS serotype IX demonstrating that the chimeric polysaccharide conjugate was as effective as the native, wild-type, IX polysaccharide conjugate.

### PS V-IXb

The mouse challenge model of GBS infection described above was used to verify the protective efficacy of PS V-IXb. Groups of eight to sixteen CD-1 female mice (age, 6-8 wk) were immunized with the new chimeric polysaccharide conjugate, non-chimeric PS V or IX, or buffer (PBS) formulated with Alum adjuvant. Protection values were calculated as [(% dead in control−% dead in vaccine)/% dead in control]×100. As shown in the table below, vaccination of mice with conjugates comprising the PS V-IXb protected mice against challenge with GBS serotype IX and V, demonstrating that this new chimeric polysaccharide conjugate was as effective against strains of the two serotypes contained in the chimera

| | | | P-val Vaccine |
|---|---|---|---|
| | Challenge with Serotype IX | | Vs Control |
| Antigen | SURVIVAL # | SURVIVAL % | (Fisher's test) |
| Control (Alum) | 15/77 | 19 | |
| WT PS conjugate | 42/45 | 93 | <0.01 |
| CRM-IX (0.25 μg PS) | | | |
| Chimeric PS | 101/111 | 91 | <0.01 |
| conjugate: CRM- | | | |
| V-IXb (0.25 μg PS) | | | |
| | Challenge with Serotype V | | |
| Antigen | SURVIVAL # | SURVIVAL % | |
| Control (Alum) | 13/56 | 23 | |
| WT PS conjugate | 15/68 | 22 | |
| CRM-IX (0.25 μg PS) | | | |
| WT PS conjugate | 59/68 | 87 | <0.01 |
| CRM-V (0.25 μg PS) | | | |
| Chimeric PS | 65/137 | 47 | <0.01 |
| conjugate: CRM- | | | |
| V-IXb (0.25 μg PS) | | | |
| WT PS conjugate | 29/114 | 25 | |
| CRM-IX (1 μg PS) | | | |
| WT PS conjugate | 109/122 | 89 | <0.01 |
| CRM-V (1 μg PS) | | | |
| Chimeric PS | 69/156 | 44 | <0.01 |
| conjugate: CRM- | | | |
| V-IXb (1 μg PS) | | | |

### Primers and Vectors for Production of Chimeric Polysaccharide Ia-Ib-III in Serotype Ia Background

Three plasmids have been designed to obtain a ‘divalent’ Ia-III chimeric polysaccharide and a ‘trivalent’ Ia-Ib-III chimeric-CPS expressing strain. DNA fragments, consisting of the cps operon type Ia, Ib and III-specific genes (cps 1aH, cps1bJ, cps1bK and cps3H) are amplified by PCR from S. agalactiae genomic DNA using specifically designed forward and reverse primers:

| Primer name | Sequence |
|---|---|
| cps3H-F | GCGGCGCGGCCGCAATTATAGGTGAAATATGAG |
| (SEQ ID NO: 27) | GAAATATCTAG |
| cps3H-R | GCGAAAATTCCTCCTATTAACTCATTTTTTAGCC |
| (SEQ ID NO: 28) | CTTCCCACT |
| cps1aH-F | GTTAATAGGAGGAATTTTCGCTTTAACC |
| (SEQ ID NO: 29) | |
| cps1aH-R | GCGGCGCGGCCGCTTAACTTTTCCACCTTTTAA |
| (SEQ ID NO: 30) | GTTGACT |
| cps3H-F-2 | AAGGGAAGCGGCCGCATTATAGGTGAAATATGA |
| (SEQ ID NO: 31) | GGAAATATCTAGATT |
| cps3H-R-2 | AAGGTAATTGAGTTAATCATTTTTTAGCCCTTCC |
| (SEQ ID NO: 32) | CACTA |
| cps1bJ-F | TTAACTCAATTACCTTTTAAATTATAGGAGTTGA |
| (SEQ ID NO: 33) | AAATGAATTATAGTATCA |
| cps1bJ-R | AGCCTTTTATAGATCTTTAGTCACTTTGTTTCTC |
| (SEQ ID NO: 34) | TCCTCCA |
| cps1bJK-R | AGCCTTTTATAGATCTTCATTTAATATCCTTCAG |
| (SEQ ID NO: 35) | ATCTACTAAAGT |

The following reaction cycle is used: 1′ at 98° C.; 10″ at 98° C., 20″ at 55° C., 3′ at 72° C. (30 cycles); 7′ at 72° C.

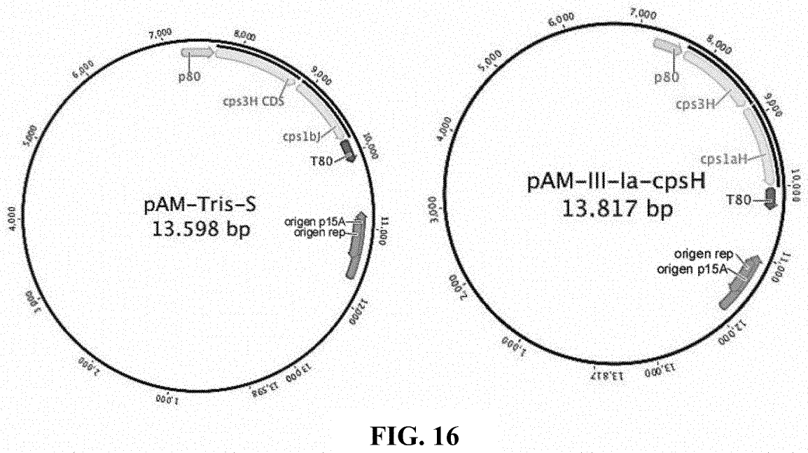

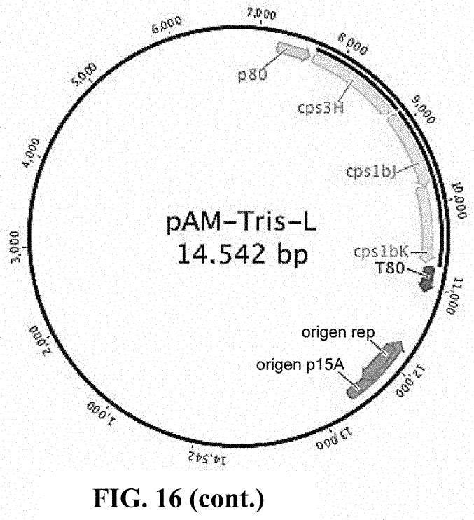

The resulting fragments (cps3H-cps1bJ-cps1bK insert, SEQ ID NO: 49; cps3H-cps1bj insert, SEQ ID NO: 51; cps3H-cps1aH insert, SEQ ID NO: 47) are ligated into the expression vector pAM-p80 (145) (SEQ ID NO: 44) to obtain plasmids pAM-Tris-L (SEQ ID NO: 48), pAM-Tris-S(SEQ ID NO: 50) and pAM-III-Ia-cpsH (SEQ ID NO: 46). The plasmids are purified from a selected clone, sequenced, and used to transform electrocompetent GBS serotype Ia cells (strain 090) by electroporation at 1,800 V as previously described (146). The vectors are shown in FIG. 16. Cells transformed with either pAM-Tris-L or pAM-Tris-S produce a chimeric polysaccharide comprising repeating units of serotypes Ia, Ib and III. Cells transformed with pAM-III-Ia-cpsH produce a chimeric polysaccharide comprising repeating unit of serotypes Ia and III. Representative sequences of cps1aH, cps3H, cps1bJ, cps1bK are provided in SEQ ID NOs: 36, 37, 38 and 39 respectively.

While certain embodiments of the present invention have been described and specifically exemplified above, it is not intended that the invention be limited to such embodiments. Various modifications may be made thereto without departing from the scope and spirit of the present invention as set forth in the following claims.

### REFERENCES

[1] Paoletti et al. (1990) J Biol Chem 265:18278-83.
[2] Wessels et al. (1990) J Clin Invest 86:1428-33.
[3] Paoletti et al. (1992) Infect Immun 60:4009-14.
[4] Paoletti et al. (1992) J Clin Invest 89:203-9.
[5] Wessels et al. (1987) Proc Nall Acad Sci USA 84:9170-4.
[6] Wang et al. (2003) Vaccine 21:1112-7.
[7] Wessels et al. (1993) Infect Immun 61:4760-6
[8] Wessels et al. (1995) J Infect Dis 171:879-84.
[9] Baker et al. (2004) J infect Dis 189:1103-12.
[10] Paoletti & Kasper (2003) Expert Opin Biol Ther 3:975-84.
[11] www.polymer.de
[12] Lewis et al. (2004) PNAS USA 101:11123-8.
[13] Wessels et al. (1989) Infect Immun 57:1089-94.
[14] WO2006/082527.
[15] US patent application U.S. 61/008,941, entitled “FERMENTATION PROCESSES FOR CULTIVATING STREPTOCOCCI AND PURIFICATION PROCESSES FOR OBTAINING CPS THEREFROM” filed on 20 Dec. 2007 and international patent application WO 2009/081276.
[16] Ramsay et al. (2001) Lancet 357(9251):195-196.
[17] Lindberg (1999) Vaccine 17 Suppl 2:S28-36.
[18] Buttery & Moxon (2000) J R Coll Physicians Lond 34:163-68.
[19] Ahmad & Chapnick (1999) Infect Dis Clin North Am 13:113-33, vii.
[20] Goldblatt (1998) J. Med. Microbiol. 47:563-7.
[21] European patent 0477508.
[22] U.S. Pat. No. 5,306,492.
[23] WO98/42721.
[24] Dick et al. in Conjugate Vaccines (eds. Cruse et al.) Karger, Basel, 1989, 10:48-114.
[25] Hermanson Bioconjugate Techniques, Academic Press, San Diego (1996) ISBN: 0123423368.
[26] WO2005/000346
[27] Anonymous (January 2002) Research Disclosure, 453077.
[28] Anderson (1983) Infect Immun 39(1):233-238.
[29] Anderson et al. (1985) J Clin Invest 76(1):52-59.
[30] EP-A-0372501.
[31] EP-A-0378881.
[32] EP-A-0427347.
[33] WO93/17712
[34] WO94/03208.
[35] WO98/58668.
[36] EP-A-0471177.
[37] WO91/01146
[38] Falugi et al. (2001) Eur J Immunol 31:3816-24.
[39] Baraldo et al. (2004) Infect Immun 72:4884-87.
[40] EP-A-0594610.
[41] WO00/56360.
[42] WO02/091998.
[43] Kuo et al. (1995) Infect Immun 63:2706-13.
[44] WO01/72337
[45] WO00/61761.
[46] WO00/33882
[47] U.S. Pat. No. 4,356,170.
[48] WO2006/082530.
[49] WO96/40242.
[50] Lei et al. (2000) Dev Biol (Basel) 103:259-264.
[51] WO00/38711; U.S. Pat. No. 6,146,902.
[52] International patent application PCT/IB2008/02690, ‘CONJUGATE PURIFICATION’, claiming priority from GB-0713880.3 (NOVARTIS AG), published as WO 2009/010877.
[53] WO99/24578.
[54] WO99/36544.
[55] WO99/57280.
[56] WO00/22430.
[57] Tettelin et al. (2000) Science 287:1809-1815.
[58] WO96/29412.
[59] Pizza et al. (2000) Science 287:1816-1820.
[60] WO01/52885.
[61] Bjune et al. (1991) Lancet 338(8775):1093-1096.
[62] Fukasawa et al. (1999) Vaccine 17:2951-2958.
[63] Rosenqvist et al. (1998) Dev. Biol. Stand. 92:323-333.
[64] Costantino et al. (1992) Vaccine 10:691-698.
[65] WO03/007985.
[66] Watson (2000) Pediatr Infect Dis J 19:331-332.
[67] Rubin (2000) Pediatr Clin North Am 47:269-285, v.
[68] Jedrzej as (2001) Microbiol Mol Biol Rev 65:187-207.
[69] Bell (2000) Pediatr Infect Dis J 19:1187-1188.
[70] Iwarson (1995) APMIS 103:321-326.
[71] Gerlich et al. (1990) Vaccine 8 Suppl:S63-68 & 79-80.
[72] Hsu et al. (1999) Clin Liver Dis 3:901-915.
[73] Gustafsson et al. (1996) N. Engl. J Med. 334:349-355.
[74] Rappuoli et al. (1991) TIBTECH 9:232-238.
[75] Vaccines (2004) eds. Plotkin & Orenstein. ISBN 0-7216-9688-0.
[76] WO02/02606.
[77] Kalman et al. (1999) Nature Genetics 21:385-389.
[78] Read et al. (2000) Nucleic Acids Res 28:1397-406.
[79] Shirai et al. (2000) J. Infect. Dis. 181(Suppl 3):5524-5527.
[80] WO99/27105.
[81] WO00/27994.
[82] WO00/37494.
[83] WO99/28475.
[84] Ross et al. (2001) Vaccine 19:4135-4142.
[85] Sutter et al. (2000) Pediatr Clin North Am 47:287-308.
[86] Zimmerman & Spann (1999) Am Fam Physician 59:113-118, 125-126.
[87] Dreesen (1997) Vaccine 15 Suppl:S2-6.
[88] MMWR Moab Mortal Wkly Rep 1998 Jan. 16; 47(1):12, 19.
[89] McMichael (2000) Vaccine 19 Suppl 1:S101-107.
[90] WO02/34771.
[91] Dale (1999) Infect Dis Clin North Am 13:227-43, viii.
[92] Ferretti et al. (2001) PNAS USA 98: 4658-4663.
[93] Kuroda et al. (2001) Lancet 357(9264):1225-1240; see also pages 1218-1219.
[94] Robinson & Torres (1997) Seminars in Immunology 9:271-283.
[95] Donnelly et al. (1997) Annu Rev Immunol 15:617-648.
[96] Scott-Taylor & Dalgleish (2000) Expert Opin Investig Drugs 9:471-480.
[97] Apostolopoulos & Plebanski (2000) Curr Opin Mol Ther 2:441-447.
[98] Ilan (1999) Curr Opin Mol Ther 1:116-120.
[99] Dubensky et al. (2000) Mol Med 6:723-732.
[100] Robinson & Pertmer (2000) Adv Virus Res 55:1-74.
[101] Donnelly et al. (2000) Am J Respir Crit Care Med 162(4 Pt 2):S190-193.
[102] Davis (1999) Mt. Sinai J Med. 66:84-90.
[103] Paoletti et al. (2001) Vaccine 19:2118-2126.
[104] WO00/56365.
[105] Gennaro (2000) Remington: The Science and Practice of Pharmacy. 20th edition, ISBN: 0683306472.
[106] Paoletti (2001) Vaccine 19(15-16):2118-26.
[107] WO03/009869.
[108] Almeida & Alpar (1996) J Drug Targeting 3:455-467.
[109] Agarwal & Mishra (1999) Indian J Exp Biol 37:6-16.
[110] WO00/53221.
[111] Jakobsen et al. (2002) Infect Immun 70:1443-1452.
[112] Bergquist et al. (1998) APMIS 106:800-806.
[113] Baudner et al. (2002) Infect Immun 70:4785-4790.
[114] Ugozzoli et al. (2002) J Infect Dis 186:1358-1361.
[115] Hennings et al. (2001) J Infect Dis. 183(7):1138-42. Epub 2001 Mar. 1.
[116] Lin et al. (2001) J Infect Dis. 184(8): 1022-8.
[117] Lin et al. (2004) J Infect Dis. 190(5):928-34
[118] Glezen & Alpers (1999) Clin. Infect. Dis. 28:219-224
[119] Madoff et al. (1994) J Clin Invest 94:286-92.
[120] Paoletti et al. (1994) Infect Immun 62:3236-43.
[121] WO03/093306.
[122] WO2004/018646.
[123] WO2004/041157.
[124] Adderson et al. (2003) Infection and Immunity 71(12):6857-6863.
[125] Geysen et al. (1984) PNAS USA 81:3998-4002.
[126] Carter (1994) Methods Mol Biol 36:207-23.
[127] Jameson, B A et al. 1988, CABIOS 4(1):181-186.
[128] Raddrizzani & Hammer (2000) Brief Bioinform 1(2):179-89.
[129] De Lalla et al. (1999) J. Immunol. 163:1725-29.
[130] Brusic et al. (1998) Bioinformatics 14(2): 121-30
[131] Meister et al. (1995) Vaccine 13(6):581-91.
[132] Roberts et al. (1996) AIDS Res Hum Retroviruses 12(7):593-610.
[133] Maksyutov & Zagrebelnaya (1993) Comput Appl Biosci 9(3):291-7.
[134] Feller & de la Cruz (1991) Nature 349(6311):720-1.
[135] Hopp (1993) Peptide Research 6:183-190.
[136] Welling et al. (1985) FEBS Lett. 188:215-218.
[137] Davenport et al. (1995) Immunogenetics 42:392-297.
[138] Bodanszky (1993) Principles of Peptide Synthesis (ISBN: 0387564314).
[139] Fields et al. (1997) Meth Enzymol 289: Solid-Phase Peptide Synthesis. ISBN: 0121821900.
[140] Chan & White (2000) Fmoc Solid Phase Peptide Synthesis. ISBN: 0199637245.
[141] Kullmann (1987) Enzymatic Peptide Synthesis. ISBN: 0849368413.
[142] Ibba (1996) Biotechnol Genet Eng Rev 13:197-216.
[143] WO2006/069200
[144] Rodriguez-Granger J, Alvargonzalez J C, Berardi A, Berner R, Kunze M, Hufnagel M, Melin P, Decheva A, Orefici G, Poyart C, Telford J, Efstratiou A, Killian M, Krizova P, Baldassarri L, Spellerberg B, Puertas A, Rosa-Fraile M: Prevention of group B streptococcal neonatal disease revisited. The DEVANI European project. Eur J Clin Microbiol Infect Dis 2012, 31:2097.
[145] Lauer P, Rinaudo C D, Soriani M, Margarit I, Maione D, Rosini R, Taddei A R, Mora M, Rappuoli R, Grandi G, Telford J L: Genome Analysis Reveals Pili in Group B Streptococcus. Science 2005 309:105.
[146] Framson P E, Nittayajarn A, Merry J, Youngman P, Rubens C E: New genetic techniques for group B streptococci: high-efficiency transformation, maintenance of temperature-sensitive pWV01 plasmids, and mutagenesis with Tn917. Applied and environmental microbiology 1997, 63:3539.
[147] Berti F, Campisi E, Toniolo C, Morelli L, Crotti S, Rosini R, Romano M R, Pinto V, Brogioni B, Torricelli G, Janulczyk R, Grandi G, Margarit I: Structure of the Type IX Group B Streptococcus Capsular Polysaccharide and Its Evolutionary Relationship with Types V and VII. J Biol Chem 2014, 289: 23437.
[148] Maione D, et al. (2005) Identification of a universal Group B Streptococcus vaccine by multiple genome screen. Science 309:148-150.
[149] Cieslewicz M J, Kasper D L, Wang Y, Wessels M R. Functional analysis in type Ia group B Streptococcus of a cluster of genes involved in extracellular polysaccharide production by diverse species of streptococci. J Biol Chem. 2001; 276(1):139-46. doi: 10.1074/jbc.M005702200 pmid: 11027683.
[150] Cieslewicz M J, Chaffin D, Glusman G, Kasper D, Madan A, Rodrigues S, et al. Structural and genetic diversity of group B streptococcus capsular polysaccharides. Infect Immun 2005; 73(5):3096-103. doi: 10.1128/IAI.73.5.3096-3103.2005 pmid:15845517; PubMed Central PMCID: PMC1087335.]

1. A chimeric bacterial capsular polysaccharide comprising at least one repeating unit of a first Streptococcus agalactiae (GBS) capsular polysaccharide serotype and at least one repeating unit of a second Streptococcus agalactiae (GBS) capsular polysaccharide serotype, wherein the repeating units are joined by a glycosidic bond.

2. The chimeric bacterial capsular polysaccharide of claim 1 which further comprises at least one repeating unit of a third GBS capsular polysaccharide serotype.

3. The chimeric bacterial capsular polysaccharide of claim 1 wherein the first GBS capsular polysaccharide serotype is type Ia and the second GBS capsular polysaccharide serotype is type Ib.

4. The chimeric bacterial capsular polysaccharide of claim 2, wherein the first GBS capsular polysaccharide serotype is type Ia, the second GBS capsular polysaccharide serotype is type Ib, and the third GBS capsular polysaccharide serotype is type III.

5. The chimeric bacterial capsular polysaccharide of claim 1 wherein the first GBS capsular polysaccharide serotype is type V and the second GBS capsular polysaccharide serotype is type IX.

6. The chimeric bacterial capsular polysaccharide of claim 2, wherein the first GBS capsular polysaccharide serotype is type V, the second GBS capsular polysaccharide serotype is type IX, and the third GBS capsular polysaccharide serotype is type VII.

7. The chimeric bacterial capsular polysaccharide of claim 1 wherein the ratio of the repeating units of the first and second GBS capsular polysaccharide serotypes is 1:1.

8. A conjugate comprising (i) the chimeric capsular polysaccharide of claim 1 and (ii) a carrier protein.

9. The conjugate of claim 8, wherein the carrier protein is covalently bound to the chimeric capsular polysaccharide.

10. The conjugate according to claim 8, wherein the carrier protein is covalently bound to the chimeric capsular polysaccharide with a linker.

11. The conjugate according to claim 10, wherein the linker is adipic acid dihydrazide.

12. The conjugate of claim 8, wherein the carrier protein is selected from the group consisting of tetanus toxoid, diphtheria toxoid, CRM197, GBS80, GBS67 and GBS59.

13. An immunogenic composition comprising the conjugate according to claim 1 and a pharmaceutically acceptable diluent, carrier, or excipient.

14.-16. (canceled)

17. An immunogenic composition comprising the conjugate according to claim 2 and a pharmaceutically acceptable diluent, carrier, or excipient.

18. A conjugate comprising (i) the chimeric bacterial capsular polysaccharide of claim 2 and (ii) a carrier protein.

19. The conjugate of claim 13, wherein the carrier protein is covalently bound to the chimeric bacterial capsular polysaccharide.

20. The conjugate according to claim 14, wherein the carrier protein is covalently bound to the chimeric bacterial capsular polysaccharide with a linker.

21. The conjugate according to claim 15, wherein the linker is adipic acid dihydrazide.

22. The conjugate of claim 13, wherein the carrier protein is selected from the group consisting of tetanus toxoid, diphtheria toxoid, CRM197, GBS80, GBS67 and GBS59.

23. The chimeric bacterial capsular polysaccharide of claim 2, wherein the ratio of the repeating units of the first, second and third GBS capsular polysaccharide serotypes is 1:1:1.


SEQUENCE LISTING (56 sequences)

SEQ ID NO: 1 - protein, 901 aa, Streptococcus agalactiae
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPMTGGKGILSFILIGGAMMSIAGGIYIWKRYKKSSDMSIKKD

SEQ ID NO: 2 - protein, 869 aa, Streptococcus agalactiae
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPMTGGKGILS

SEQ ID NO: 3 - protein, 858 aa, Streptococcus agalactiae
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGI

SEQ ID NO: 4 - protein, 896 aa, Streptococcus agalactiae
MRKYQKFSKILTLSLFCLSQIPLNTNVLGESTVPENGAKGKLVVKKTDDQNKPLSKATFVLKPTSHSESKVEKVTTEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGKSIIKTINKDEPLDVVFVLDNSNSMKNNGKNNKAKKAGEAVETIIKDVLGANVENRAALVTYGSDIFDGRTVKVIKGFKEDPYYGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVKGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSDGEITELMNSFSSKPEYYTPIVTSADVSNNEILSKIQQQFEKILTKENSIVNGTIEDPMGDKINLHLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEEPDTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILSFILIGGAMMSIAGGIYIWKRHKKSSDASIEKD

SEQ ID NO: 5 - protein, 864 aa, Streptococcus agalactiae
MRKYQKFSKILTLSLFCLSQIPLNTNVLGESTVPENGAKGKLVVKKTDDQNKPLSKATFVLKPTSHSESKVEKVTTEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGKSIIKTINKDEPLDVVFVLDNSNSMKNNGKNNKAKKAGEAVETIIKDVLGANVENRAALVTYGSDIFDGRTVKVIKGFKEDPYYGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVKGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSDGEITELMNSFSSKPEYYTPIVTSADVSNNEILSKIQQQFEKILTKENSIVNGTIEDPMGDKINLHLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEEPDTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILS

SEQ ID NO: 6 - protein, 853 aa, Streptococcus agalactiae
MRKYQKFSKILTLSLFCLSQIPLNTNVLGESTVPENGAKGKLVVKKTDDQNKPLSKATFVLKPTSHSESKVEKVTTEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGKSIIKTINKDEPLDVVFVLDNSNSMKNNGKNNKAKKAGEAVETIIKDVLGANVENRAALVTYGSDIFDGRTVKVIKGFKEDPYYGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVKGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSDGEITELMNSFSSKPEYYTPIVTSADVSNNEILSKIQQQFEKILTKENSIVNGTIEDPMGDKINLHLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEEPDTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGI

SEQ ID NO: 7 - protein, 554 aa, Streptococcus agalactiae
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 8 - protein, 517 aa, Streptococcus agalactiae
AEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 9 - protein, 525 aa, Streptococcus agalactiae
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTG

SEQ ID NO: 10 - protein, 520 aa, Streptococcus agalactiae
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 11 - protein, 483 aa, Streptococcus agalactiae
AEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 12 - protein, 271 aa, Streptococcus agalactiae
AEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKG

SEQ ID NO: 13 - protein, 502 aa, Streptococcus agalactiae
MKKKMIQSLLVASLAFGMAVSPVTPIAFAAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTELPSTGGIGTTIFYIIGAILVIGAGIVLVARRRLRS

SEQ ID NO: 14 - protein, 473 aa, Streptococcus agalactiae
AETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTELPSTGGIGTTIFYIIGAILVIGAGIVLVARRRLRS

SEQ ID NO: 15 - protein, 467 aa, Streptococcus agalactiae
MKKKMIQSLLVASLAFGMAVSPVTPIAFAAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTE

SEQ ID NO: 16 - protein, 438 aa, Streptococcus agalactiae
AETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTE

SEQ ID NO: 17 - protein, 736 aa, Streptococcus agalactiae
MGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPIGSGSGNKPGKKVKEIPVTPSNGEITVSKTWDKGSDLENANVVYTLKDGGTAVASVSLTKTTPNGEINLGNGIKFTVTGAFAGKFSGLTDSKTYMISERIAGYGNTITTGAGSAAITNTPDSDNPTPLGSGSGNNPTEESEPQEGTPANQEIKVIKDWAVDGTITDANVAVKAIFTLQEKQTDGTWVNVASHEATKPSRFEHTFTGLDNAKTYRVVERVSGYTPEYVSFKNGVVTIKNNKNSNDPTPIGSGSGNKPGKDLTELPVTPSKGEVTVAKTWSDGIAPDGVNVVYTLKDKDKTVASVSLTKTSKGTIDLGNGIKFEVSGNFSGKFTGLENKSYMISERVSGYGSAINLENGKVTITNTKDSDNPTPLGSGSGNKPGTDLSEQPVTPEDGEVKVTKTWAAGANKADAKVVYTLKNATKQVVASVALTAADTKGTINLGKGMTFEITGAFSGTFKGLQNKAYTVSERVAGYTNAINVTGNAVAITNTPDSDNPTPLGSGSGNNPTTENEPQTGNPVNKEITVRKTWAVDGNEVNKGDEKVDAVFTLQVKDSDKWVNVDSATATAATDFKYTFKNLDNAKTYRVVERVSGYAPAYVSFVGGVVTIKNNKNSNDPTPI

SEQ ID NO: 18 - protein, 1378 aa, Streptococcus agalactiae
MGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPINPSEPKVVTYGRKFVKTNKDGKERLAGATFLVKKDGKYLARKSGVATDAEKAAVDSTKSALDAAVKAYNDLTKEKQEGQDGKSALATVSEKQKAYNDAFVKANYSYEGSGSGNNPTEESEPQEGTPANQEIKVIKDWAVDGTITDANVAVKAIFTLQEKQTDGTWVNVASHEATKPSRFEHTFTGLDNAKTYRVVERVSGYTPEYVSFKNGVVTIKNNKNSNDPTPINPSEPKVVTYGRKFVKTNQANTERLAGATFLVKKEGKYLARKAGAATAEAKAAVKTAKLALDEAVKAYNDLTKEKQEGQEGKTALATVDQKQKAYNDAFVKANYSYEGSGSGNKPGTDLSEQPVTPEDGEVKVTKTWAAGANKADAKVVYTLKNATKQVVASVALTAADTKGTINLGKGMTFEITGAFSGTFKGLQNKAYTVSERVAGYTNAINVTGNAVAITNTPDSDNPTPLNPTQPKVETHGKKFVKVGDADARLAGAQFVVKNSAGKFLALKEDAAVSGAQTELATAKTDLDNAIKAYNGLTKAQQEGADGTSAKELINTKQSAYDAAFIKARTAYTGSGSGNKPGKDLTELPVTPSKGEVTVAKTWSDGIAPDGVNVVYTLKDKDKTVASVSLTKTSKGTIDLGNGIKFEVSGNFSGKFTGLENKSYMISERVSGYGSAINLENGKVTITNTKDSDNPTPLNPTEPKVETHGKKFVKTNEQGDRLAGAQFVVKNSAGKYLALKADQSEGQKTLAAKKIALDEAIAAYNKLSATDQKGEKGITAKELIKTKQADYDAAFIEARTAYEGSGSGNKPGKKVKEIPVTPSNGEITVSKTWDKGSDLENANVVYTLKDGGTAVASVSLTKTTPNGEINLGNGIKFTVTGAFAGKFSGLTDSKTYMISERIAGYGNTITTGAGSAAITNTPDSDNPTPLNPTEPKVVTHGKKFVKTSSTETERLQGAQFVVKDSAGKYLALKSSATISAQTTAYTNAKTALDAKIAAYNKLSADDQKGTKGETAKAEIKTAQDAYNAAFIVARTAYEGSGSGNNPTTENEPQTGNPVNKEITVRKTWAVDGNEVNKGDEKVDAVFTLQVKDSDKWVNVDSATATAATDFKYTFKNLDNAKTYRVVERVSGYAPAYVSFVGGVVTIKNNKNSNDPTPINPSEPKVVTYGRKFVKTNQDGSERLAGATFLVKNSQSQYLARKSGVATNEAHKAVTDAKVQLDEAVKAYNKLTKEQQESQDGKAALNLIDEKQTAYNEAFAKANYSYE

SEQ ID NO: 19 - protein, 916 aa, Streptococcus agalactiae
MGNKPGKDLTELPVTPSKGEVTVAKTWSDGIAPDGVNVVYTLKDKDKTVASVSLTKTSKGTIDLGNGIKFEVSGNFSGKFTGLENKSYMISERVSGYGSAINLENGKVTITNTKDSDNPTPLNPTEPKVETHGKKFVKTNEQGDRLAGAQFVVKNSAGKYLALKADQSEGQKTLAAKKIALDEAIAAYNKLSATDQKGEKGITAKELIKTKQADYDAAFIEARTAYEGSGSGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPINPSEPKVVTYGRKFVKTNKDGKERLAGATFLVKKDGKYLARKSGVATDAEKAAVDSTKSALDAAVKAYNDLTKEKQEGQDGKSALATVSEKQKAYNDAFVKANYSYEGSGSGNKPGKKVKEIPVTPSNGEITVSKTWDKGSDLENANVVYTLKDGGTAVASVSLTKTTPNGEINLGNGIKFTVTGAFAGKFSGLTDSKTYMISERIAGYGNTITTGAGSAAITNTPDSDNPTPLNPTEPKVVTHGKKFVKTSSTETERLQGAQFVVKDSAGKYLALKSSATISAQTTAYTNAKTALDAKIAAYNKLSADDQKGTKGETAKAEIKTAQDAYNAAFIVARTAYEGSGSGNNPTEESEPQEGTPANQEIKVIKDWAVDGTITDANVAVKAIFTLQEKQTDGTWVNVASHEATKPSRFEHTFTGLDNAKTYRVVERVSGYTPEYVSFKNGVVTIKNNKNSNDPTPINPSEPKVVTYGRKFVKTNQANTERLAGATFLVKKEGKYLARKAGAATAEAKAAVKTAKLALDEAVKAYNDLTKEKQEGQEGKTALATVDQKQKAYNDAFVKANYSYE

SEQ ID NO: 20 - protein, 916 aa, Streptococcus agalactiae
MGNNPTEESEPQEGTPANQEIKVIKDWAVDGTITDANVAVKAIFTLQEKQTDGTWVNVASHEATKPSRFEHTFTGLDNAKTYRVVERVSGYTPEYVSFKNGVVTIKNNKNSNDPTPINPSEPKVVTYGRKFVKTNQANTERLAGATFLVKKEGKYLARKAGAATAEAKAAVKTAKLALDEAVKAYNDLTKEKQEGQEGKTALATVDQKQKAYNDAFVKANYSYEGSGSGNKPGKKVKEIPVTPSNGEITVSKTWDKGSDLENANVVYTLKDGGTAVASVSLTKTTPNGEINLGNGIKFTVTGAFAGKFSGLTDSKTYMISERIAGYGNTITTGAGSAAITNTPDSDNPTPLNPTEPKVVTHGKKFVKTSSTETERLQGAQFVVKDSAGKYLALKSSATISAQTTAYTNAKTALDAKIAAYNKLSADDQKGTKGETAKAEIKTAQDAYNAAFIVARTAYEGSGSGNKPGKDLTELPVTPSKGEVTVAKTWSDGIAPDGVNVVYTLKDKDKTVASVSLTKTSKGTIDLGNGIKFEVSGNFSGKFTGLENKSYMISERVSGYGSAINLENGKVTITNTKDSDNPTPLNPTEPKVETHGKKFVKTNEQGDRLAGAQFVVKNSAGKYLALKADQSEGQKTLAAKKIALDEAIAAYNKLSATDQKGEKGITAKELIKTKQADYDAAFIEARTAYEGSGSGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPINPSEPKVVTYGRKFVKTNKDGKERLAGATFLVKKDGKYLARKSGVATDAEKAAVDSTKSALDAAVKAYNDLTKEKQEGQDGKSALATVSEKQKAYNDAFVKANYSYE

SEQ ID NO: 21 - protein, 1227 aa, Streptococcus agalactiae
MDGSILADSKAVPVKITLPLVNDNGVVKDAHVYPKNTETKPQVDKNFADKELDYANNKKDKGTVSASVGDVKKYHVGTKILKGSDYKKLIWTDSMTKGLTFNNDIAVTLDGATLDATNYKLVADDQGFRLVLTDKGLEAVAKAAKTKDVEIKITYSATLNGSAVVEVLETNDVKLDYGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPINPSEPKVVTYGRKFVKTNKDGKERLAGATFLVKKDGKYLARKSGVATDAEKAAVDSTKSALDAAVKAYNDLTKEKQEGQDGKSALATVSEKQKAYNDAFVKANYSYEGSGSNGSLLAASKAVPVNITLPLVNEDGVVADAHVYPKNTEEKPEIDKNFAKTNDLTALTDVNRLLTAGANYGNYARDKATATAEIGKVVPYEVKTKIHKGSKYENLVWTDIMSNGLTMGSTVSLKASGTTETFAKDTDYELSIDARGFTLKFTADGLGKLEKAAKTADIEFTLTYSATVNGQAIIDNPESNDIKLSYGNKPGKDLTELPVTPSKGEVTVAKTWSDGIAPDGVNVVYTLKDKDKTVASVSLTKTSKGTIDLGNGIKFEVSGNFSGKFTGLENKSYMISERVSGYGSAINLENGKVTITNTKDSDNPTPLNPTEPKVETHGKKFVKTNEQGDRLAGAQFVVKNSAGKYLALKADQSEGQKTLAAKKIALDEAIAAYNKLSATDQKGEKGITAKELIKTKQADYDAAFIEARTAYEGSGSNGSILADSKAVPVKITLPLVNNQGVVKDAHIYPKNTETKPQVDKNFADKDLDYTDNRKDKGVVSATVGDKKEYIVGTKILKGSDYKKLVWTDSMTKGLTFNNNVKVTLDGEDFPVLNYKLVTDDQGFRLALNATGLAAVAAAAKDKDVEIKITYSATVNGSTTVEIPETNDVKLDYGNNPTEESEPQEGTPANQEIKVIKDWAVDGTITDANVAVKAIFTLQEKQTDGTWVNVASHEATKPSRFEHTFTGLDNAKTYRVVERVSGYTPEYVSFKNGVVTIKNNKNSNDPTPINPSEPKVVTYGRKFVKTNQANTERLAGATFLVKKEGKYLARKAGAATAEAKAAVKTAKLALDEAVKAYNDLTKEKQEGQEGKTALATVDQKQKAYNDAFVKANYSYE

SEQ ID NO: 22 - protein, 1179 aa, Streptococcus agalactiae
MGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPIGSGSGNKPGKKVKEIPVTPSNGEITVSKTWDKGSDLENANVVYTLKDGGTAVASVSLTKTTPNGEINLGNGIKFTVTGAFAGKFSGLTDSKTYMISERIAGYGNTITTGAGSAAITNTPDSDNPTPLGSGSGNNPTEESEPQEGTPANQEIKVIKDWAVDGTITDANVAVKAIFTLQEKQTDGTWVNVASHEATKPSRFEHTFTGLDNAKTYRVVERVSGYTPEYVSFKNGVVTIKNNKNSNDPTPIGSGSGNKPGKDLTELPVTPSKGEVTVAKTWSDGIAPDGVNVVYTLKDKDKTVASVSLTKTSKGTIDLGNGIKFEVSGNFSGKFTGLENKSYMISERVSGYGSAINLENGKVTITNTKDSDNPTPLGSGSGNKPGTDLSEQPVTPEDGEVKVTKTWAAGANKADAKVVYTLKNATKQVVASVALTAADTKGTINLGKGMTFEITGAFSGTFKGLQNKAYTVSERVAGYTNAINVTGNAVAITNTPDSDNPTPLGSGSGNNPTTENEPQTGNPVNKEITVRKTWAVDGNEVNKGDEKVDAVFTLQVKDSDKWVNVDSATATAATDFKYTFKNLDNAKTYRVVERVSGYAPAYVSFVGGVVTIKNNKNSNDPTPIGSGGGGETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTE

SEQ ID NO: 23 - DNA, 37 bp, Streptococcus agalactiae
gcggcgcggccgcgacatatttgctctgatatggcag

SEQ ID NO: 24 - DNA, 35 bp, Streptococcus agalactiae
gcggcagatctgggataatgatactaatcatcttc

SEQ ID NO: 25 - DNA, 37 bp, Streptococcus agalactiae
gcggcgcggccgcgctctgatatggcaggaggtaagg

SEQ ID NO: 26 - DNA, 35 bp, Streptococcus agalactiae
gcggcagatctgggataatgatactaactttatcc

SEQ ID NO: 27 - DNA, 44 bp, Streptococcus agalactiae
gcggcgcggccgcaattataggtgaaatatgaggaaatatctag

SEQ ID NO: 28 - DNA, 43 bp, Streptococcus agalactiae
gcgaaaattcctcctattaactcattttttagcccttcccact

SEQ ID NO: 29 - DNA, 28 bp, Streptococcus agalactiae
gttaataggaggaattttcgctttaacc

SEQ ID NO: 30 - DNA, 40 bp, Streptococcus agalactiae
gcggcgcggccgcttaacttttccaccttttaagttgact

SEQ ID NO: 31 - DNA, 48 bp, Streptococcus agalactiae
aagggaagcggccgcattataggtgaaatatgaggaaatatctagatt

SEQ ID NO: 32 - DNA, 39 bp, Streptococcus agalactiae
aaggtaattgagttaatcattttttagcccttcccacta

SEQ ID NO: 33 - DNA, 52 bp, Streptococcus agalactiae
ttaactcaattaccttttaaattataggagttgaaaatgaattatagtatca

SEQ ID NO: 34 - DNA, 41 bp, Streptococcus agalactiae
agccttttatagatctttagtcactttgtttctctcctcca

SEQ ID NO: 35 - DNA, 46 bp, Streptococcus agalactiae
agccttttatagatcttcatttaatatccttcagatctactaaagt

SEQ ID NO: 36 - DNA, 1183 bp, Streptococcus agalactiae
gttaataggaggaattttcgctttaaccctattttcaaagccaatgcaacttttgttacttttagcattaatagttttacttatttgtagtagttataatgaaaaaatgaaatttttaaatatggctgaaatttttttcattgtattttatattatttatttaacttcaatattgctacattctttgtttaaaaatcctgattttgatagaattttagcagcttttaactcgttgattatcggtatagtatcattggctttttatcattattataagaatacaactttggagttagataaaatattaaaagcatttttatttaatgggttaatcctattttttttagggggaacatattattattgtttgcataataatattcaaaatatcagtatttttggtagagatttgattgggtcagactggattaatggtatgcatactcaaagagcaatgggattttttgaatattcaaaccttataattcctattacagtggtaactaacttatatatatatatatatatgaagttaagaaactattcaattatgaccataggtgttgtattattatttacctttattttacctattggatcgggctccagggctggaatagtagctatattggcgcagatgtttattcttcttctaaatacagttgtcgtaaagaagaaaactataaaatttttattgtacatacttccgtttctactagtaatagtaatgatgttatattttgataacttactatctatatattatcgtataattaatttgcgatccgggagtagtgaatccagattttctttatataaagatacagtaaacatcgttataaataattctttattatttggagaaggagttaaagagttatggttaaatagtgatctacctttggggtcgcattcaacgtatataggctatttctacaaaagtggcctgctgggattaatgaatatagttccaggtttgcttttaatttttactaatattggtaggaaagctaaacaatcagctttttattatgagatagtaggaacacttataactttattctcattttttgcacttgaagatcttgacggagctaattggcttattgtttttatttttacagtgttaggaattttagaaaataaggatttttatagtcaacttaaaaggtggaaaagttaa

SEQ ID NO: 37 - DNA, 2369 bp, Streptococcus agalactiae
aattataggtgaaatatgaggaaatatctagatttagattattctttattttatgctctttgggtacttattttagtaccaaaccaatggtatcagtttttaattattaccattatagttctattattactttggaagagtgagtttagaatatctataagcaattcttcaatactatttctgctttggttatttatttatttatttgcaatactcattagaggtactcaagaggatataacgtttcagcgatttattgctgagctattaaaactaattagtacaggatatgctttatttttttataattattatagaaaagctgattttaatagttcagttgtaaggaatgtggtaaaggttaactattttgtgttgtttcttataacagttttatatttattttttcctatgctgaagccaactttatttggaagagaattgttttcaatagagtggtttccacatatgagaataagacttgcggcatattttgaatatgctacactaattggtcagtttattttattttcttatcccatactttttttgaaaccccaaaaacatatggaaaatattttaatatccttactgttgactatatgttcatacttttctggcgctagaatactattggtctgtatgttggttttattagcatcgcttcttttagattatatcctttttaaaactaatttgaaattgaccaagaaaaacacttttatacttggtatgactttcttatttatcaccgcttgtttttcttataacatatggtcaataattgaaaaaataattatgtacagaaaccaaagtactatcactaggatgatagtttatcaagaaagtattattgaagttctaaaaggaaatattttatttggacagggtataaggattccatcaagtgaaggaatattcctaggatcgcattctacttatattagtgtcttttacaggacttctttattaggaattgttctttatttttctgcctttatacttttatataaagaagcgatttcaaaaaattataaaatctacagattatttttttatacgttattatgttacacgctctttgaggaaatagatcctaatcattggagtattgtattattattctcaacttttggtatagtgggaagggctaaaaaatgagttaataggaggaattttcgctttaaccctattttcaaagccaatgcaacttttgttacttttagcattaatagttttacttatttgtagtagttataatgaaaaaatgaaatttttaaatatggctgaaatttttttcattgtattttatattatttatttaacttcaatattgctacattctttgtttaaaaatcctgattttgatagaattttagcagcttttaactcgttgattatcggtatagtatcattggctttttatcattattataagaatacaactttggagttagataaaatattaaaagcatttttatttaatgggttaatcctattttttttagggggaacatattattattgtttgcataataatattcaaaatatcagtatttttggtagagatttgattgggtcagactggattaatggtatgcatactcaaagagcaatgggattttttgaatattcaaaccttataattcctattacagtggtaactaacttatatatatatatatatatgaagttaagaaactattcaattatgaccataggtgttgtattattatttacctttattttacctattggatcgggctccagggctggaatagtagctatattggcgcagatgtttattcttcttctaaatacagttgtcgtaaagaagaaaactataaaatttttattgtacatacttccgtttctactagtaatagtaatgatgttatattttgataacttactatctatatattatcgtataattaatttgcgatccgggagtagtgaatccagattttctttatataaagatacagtaaacatcgttataaataattctttattatttggagaaggagttaaagagttatggttaaatagtgatctacctttggggtcgcattcaacgtatataggctatttctacaaaagtggcctgctgggattaatgaatatagttccaggtttgcttttaatttttactaatattggtaggaaagctaaacaatcagctttttattatgagatagtaggaacacttataactttattctcattttttgcacttgaagatcttgacggagctaattggcttattgtttttatttttacagtgttaggaattttagaaaataaggatttttatagtcaacttaaaaggtggaaaagttaagcggccgcatcgatcgatgagatct

SEQ ID NO: 38 - DNA, 942 bp, Streptococcus agalactiae
atgaattatagtatcattatgtcggtatataatgagcctttaaattatgtgagagattcagtagaatctatattaaatcaaacgcttactgattttgagttcataattgtcattgataatccaagtagaggtgatttaaagcaattcttaacagaatattcagttgtagataatagaataaaaatcttgcttaatgaagaaaatattggtttagcatcaagtttgaacaaagcggtgaaaatttctaagggagaatatatttttagaatggatgctgatgatatttcatatccaagtagatttgataagcaaattcgttttatggaggaaaattcattggatttctcagcaactctaatagaattgatagaccaaaaaggaaatttagtatataaacaacgagaaagtaataaaatatacttaactaatgatatacggaagatgttattgaatagatctatacttgcccacccaacgtggtgcgtaaaaaagaaagttttcgataagttaatgggatatagagatttagtacctgttgaagattatgattttgcaataagaggagctctggctgatttcaaaatcggcttactcaataaagtacttttacagtatagattaaacgagaatggaatatcacaaaccaataagtttaagcaatatatttactcagctattttacaagatttttataaagaaaaatcttatattgatatcacaaaaattactaattactttcaagagtatgtgataaagaaacgctatactcagcaagagctctctaaatattttgagctaaaatctacccctagtattactattagaaaactatatatttgtttatatttatactttaagtctcccttggttaggaggttattaataaatgatattaatattttagtactgaaattgtttggaggagagaaacaaagtgactaa

SEQ ID NO: 39 - DNA, 939 bp, Streptococcus agalactiae
gtgactaatagaaaaatttatgtatgtcatactctttatcatttattgatttgtttatataaagaagagatatattcaaatttagaaattattctctcttcttctattcctgatgttgataatttagagaaaaaattaaaatcaaaaacaataaatatacatattttagaagaatctagtggtgaaagtgaagaattattatcagtacttaaagatgctggtctaagttatagtaagtttgatagtaattgttttatttttaatgatgcaacgcctattgggaggacactaataaagcatggtatttattataatctaattgaagatggtttaaattgttttacttactctatatttagtcaaaaactttggaagtattatgtaaaaaaatatattcttcacaaaattcagccacatggattttcacgatattgtttagggattgaagttaattcattagttaatttgccaaaggatccgcgttataaaaaatttattgaagtccctaggaaagaactttttgacaatgtaacagaatatcaaaaagaaatggcaataaatctttttggagcagtaagagttagtattaaatcaccttcagtactagtattaacgcagcctctatctatagataaagagtttatgagttataacaataagatagaaacgtccgaagaacaatttaatttttataaatcaatagtcaatgaatatataaataaagggtacaatgtttatttaaaagttcatcctagagatgtagtagattattccaaattgccggtagagctattaccatcaaatgttcctatggaaattatagagttgatgttaacaggtcggttcgaatgtgggataacacattcgtccactgcgctggattttttaacttgtgttgataaaaaaataactttagtagatctgaaggatattaaatga

SEQ ID NO: 40 - DNA, 726 bp, Streptococcus agalactiae
atgatacctaaagttatacattattgttggtttggaggaaatcccttaccagataatttaaagaaatatataaaaacttggagagaacaatgtccggattatgaaattattgaatggaatgagcataattatgatgttagtaaaaatgtttttatgagagaagcatatactaagaagaattttgcttatgtttctgactatgcaagattggatattatttatacttatggggggttctatctagatactgatgtggagcttttaaaaagtttagatcctttgaggattcatgagtgttttctagcaagggagattagttgtgatgtgaatacaggattaataattggcgctgttaaaggacatcactttttaaaatcaaatatgtctatatatgacaaaagtgatttaacttctcttaataagacatgtgtagaggttacaactaatttattgataaacagagggcttaagaataagaatattattcaaaagattgatgatataacaatatatccgagaaattattttaatccaaagaatttattaacaggtaaggttgattgtctgactagtgttacctattctatacatcattacgaaggaagttggaaaagttcttcatttatttcagattctctaaagattagagtaaggctcataattgattttttatttggatatggtacttatagaatgcttctaaggtttctaaagttaaagaaatag

SEQ ID NO: 41 - DNA, 984 bp, Streptococcus agalactiae
atggaattaatttcaataatagtgcctgtatataatggagaaatatatattgggagatgtctagatagtattcttgagcaaacttatcagaatttagagattatcataattgatgatggctctagtgatcgcacaggggatatttgcgaaaagtattttttggaagacaggcgaataaaatatttctatcaagaaaacagaggccaatcagttgcacgtaacaacggagtacttaggtgtacaggagattggattgcttttttagacagtgatgatgtttatcttccttattcaattgaggttatgtacaacatacagaaagctaccaatgccgatattgttttaactagtattggtaacttcaacaatacttacaatactagtattaattcacagtatttaaaagagattaaactttatactttagaggtggcgttggaagaaatgtattatgggaaaacttatggagtttccccgttagcaaaattatatccgagaagtaatttattgagtaatccatatccagaaggaaaaattcatgaagatatggatacaacttttaagcttatatcttgcgcttcaaaaatagcagtttgtgatattgtcactgcagtggtgtattttagcgataatagtactactaggacaaaattcaatgagagaatgttgtatttttttgaggcaattcaaaataatattgtatttataaatctaaattttccacataatacatctttaatctctgctgtaatatataatgaagtatttggtggaatagatatatgtgggaagatgattgattttaaattatatgatacagttgattattacagaaaaaaatatagaaaatattttaagactattctctttaataataggattagtgtgaaggagaaggttaaatatatattatttatttcctctataagatatttcactattgtaagaaaaatatataatttgaggttatcaaggaatggataa

SEQ ID NO: 42 - DNA, 1073 bp, Streptococcus agalactiae
ggatattgcgttttttaaaattaaaatagtagcagttattaatttaaaatattctctttaaaaaatggaggcacaatgaaagtatcattagttataccagtttataatggagcaaatcatatcaaagaaacattagaaagtattatagcgcagacctaccccaatttggaaatattaatcattgatgatgggtcaactgataatacatcaactatatgtcagaagtatgtttctaaagataagcgcatgaaatatatatataaaaaaaatggaggtcaatcttctgctagaaatttaggcgttaaactatctacaagtaattggatagctttcttagattctgatgatctttgttcgccattttatatagaatgtctagtgagaattcaacatgaaactggtgctgaccttgtaataactggaattgaggatcacccttgttacagaacggcaaaactttcatttgataaactatcttactcagtactcagtgtggaagagacactaatagaaatgtattatgataactatggtatttctccattagggaagttatattccaaagggttattattaaaatatccataccctattggtaaaattcatgaagatacagacacaacttataaactgattgcaagtagtacacgaattgcattgagcaaagagaaaacagtatatgtcaaagggcaacctaatagtactaccagacagtcatacaaagagaatatgttatattattttgaagcaatgcaacataatagagattttatccttgaacattattcagataaagaactaattgatgctttaaattataaaactgtaattggaggaatgtggatcgttaataagcttttagaaggagataagtacgattatattaattctttgagaagtgaatatagaaaatattataagagtatattatttaataagcgagttacaattaaagaaaaaataaaatatttttcatttatttcgtcaatgaatttatttttatttttaaaaaaattttataactataatgggagataaattatgaagatgattagtatcattatcccagatct

SEQ ID NO: 43 - DNA, 1815 bp, Streptococcus agalactiae
gcggccgcgacatatttgctctgatatggcaggaggtaaggaaggaaaatgatacctaaagttatacattattgttggtttggaggaaatcccttaccagataatttaaagaaatatataaaaacttggagagaacaatgtccggattatgaaattattgaatggaatgagcataattatgatgttagtaaaaatgtttttatgagagaagcatatactaagaagaattttgcttatgtttctgactatgcaagattggatattatttatacttatggggggttctatctagatactgatgtggagcttttaaaaagtttagatcctttgaggattcatgagtgttttctagcaagggagattagttgtgatgttgccactggattaattattggtgcagttagaggacaaaaatttataaaagataatatgatggtatatgaaaaatataaatttttaaattttgataaaacttgtgtggaaattacgactgacttacttttaaaaaaaggattgaagaataaagatgtcatacaagttatagatggtgttactatttatccaagacaatattttaatcctaaaagtatgttgacggggaggttagattttctaacagatgatacatactctatacaccattatgaaggaagctggaaaaaaagatcatttgttcctgattccctaaagattagattacgtcttataatcgattttgtgttcggatatggaacatatagaaggatattgcgttttttaaaattaaaatagtagcagttattaatttaaaatattctctttaaaaaatggaggcacaatgaaagtatcattagttataccagtttataatggagcaaatcatatcaaagaaacattagaaagtattatagcgcagacctaccccaatttggaaatattaatcattgatgatgggtcaactgataatacatcaactatatgtcagaagtatgtttctaaagataagcgcatgaaatatatatataaaaaaaatggaggtcaatcttctgctagaaatttaggcgttaaactatctacaagtaattggatagctttcttagattctgatgatctttgttcgccattttatatagaatgtctagtgagaattcaacatgaaactggtgctgaccttgtaataactggaattgaggatcacccttgttacagaacggcaaaactttcatttgataaactatcttactcagtactcagtgtggaagagacactaatagaaatgtattatgataactatggtatttctccattagggaagttatattccaaagggttattattaaaatatccataccctattggtaaaattcatgaagatacagacacaacttataaactgattgcaagtagtacacgaattgcattgagcaaagagaaaacagtatatgtcaaagggcaacctaatagtactaccagacagtcatacaaagagaatatgttatattattttgaagcaatgcaacataatagagattttatccttgaacattattcagataaagaactaattgatgctttaaattataaaactgtaattggaggaatgtggatcgttaataagcttttagaaggagataagtacgattatattaattctttgagaagtgaatatagaaaatattataagagtatattatttaataagcgagttacaattaaagaaaaaataaaatatttttcatttatttcgtcaatgaatttatttttatttttaaaaaaattttataactataatgggagataaattatgaagatgattagtatcattatcccagatct

SEQ ID NO: 44 - DNA, 11465 bp, Streptococcus agalactiae
gtgacatgactgaaataggtagttgagatatggtatgcaatgtttgaacatgtttaaaatcgaatgtaaccatttgatagaccgccttcattatcatttctagaatttttctttaggtttgtaaagactacaaaataaaatgatgaaaacaactatcttgtggatacactaaaaagacacgctaattagcaaactctctcttcatcatctctcaccattattatactactatttatatgacaaataaaggttattttgttaaaatataactttgaaaatccacatatatttttaatcttccgtctgaaaaaataaataaaaatagtaaaaataaacacgaatttaaaataagcaaattttttaagaaaatctgtgctaaactttaatagttttgtgcttaataataatcagcacttacaaagaacaaagggaagcggccgcatcgatcgatgagatctataaaaggctacttcttaagtaaccatgtttaagaaaagagaaatagccttatttctctttttgtcgtttttaaaataaaggaacatcatgaaacaaacattaaaacttatgttttcttttctgttgatgttagggactatgtttggaattagccaaactgttttagcgcaagaaactcatcagttgacgattgttcatcttgaagcaagggatattgatcgtccaaatccacagttggagattgcccctaaagaagggactccaattgaaggagtactctatcagttgtaccaattaaaatcaactgaagatggcgatgtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgactttaaacgtggatcattttctttaaatttatgctgacgacctttgaatttgccttttttcttagcaatttcgattccttgtgcctgacgttccttaattttttttcgttctgattctgcttgatacttgtacaattcaatgacaaggctattaatcaaacgccttaaattttcatcttcaataccattcattgagggtaaatttaagacttccagggttgcccccttaatttgaatttgattcatcaattctgttaattctttattatttcgtcctaatcgatctaattcagtaacaataacaatatccccttcacgaatatagttaagcatagcttgtaattgtgggcgttcgaccgattgaccgcttaatttgtctgaaaagaccttagaaacgccctgtaacgcttgtaattgccgatctaagttctgttctttgctactgacacgtgcataaccaattttagccattttcaaccaacctctaaaattctctcggttgcaataaccaatcagcaatatctactttttcaatttcaaattgcttatcagaaattgtcttttcgtaagcgataaaatcttgcgcatattgttgctcattaaaaatagccaccacttcgtcattttctaaaactcgataaataaattttttcattttactcctcctattatgcccaacttaaatgacctattcaccaagtcaattatactgctaaaatcatattaggacaaataggtatactctattgacctataaatgatagcaacttaaaagatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcactaaacagcaaagaaaaacaaacacgtatcatagatataaatgtaatggcatagtgcgggttttattttcagcctgtatcgtagctaaacaaatcgagttgtgggtccgttttggggcgttctgccaatttgtttagagtttcttgaataaatgtacgttctaaattaaacgaagctgtcagcgcctttatatagctttctcgttcttctttttttaatttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcgctaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacataacgctattgcgccctactaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattgggatcaaaaaattctacattgtccgttcttggtatacgagcaatcccaaaatgattgcacgttagatcaactggcaaagactttccaaaatattctcggatattttgcgagattattttggctgctttgacagatttaaattctgattttgaagtcacatagactggcgtttctaaaacaaaatatgcttgataacctttatcagatttgataattaacgtaggcataaaacctaaatcaatagctgttgttaaaatatcgcttgctgaaatagtttctttttccgtgtgaatatcaaaatcaataaagaaggtattgatttgtcttaaattgttttcagaatgtcctttagtgtatgaacggttttcgtctgcatacgtaccataacgataaacgtttggtgtccaatgcgtaaatgtatcttgattttcgtgaatcgcttcttcggaagtcagaacaacgccacgtccgccaatcatgcttttttttgagcgatacgcaaaaatagcccctttacttttacctggcttggtagtgattgagcgaattttactatttttaaatttgtactttaacaagccgtcatgaagcacagtttctacaacaaaagggatattcattcagctgttctcctttcttacgaaaattaattagttagaagctacgatcaaagttgaatcacaacaaaaaaggcaatcaactaagtttttcttaattgattgcctggtatcttcttaaagacttgaaatcccctcaaaaacccgatataatgggtttacagatatttaagtatctgattaataaagtaattaaatactttaccaaattttgggtctcgacttctttaattgattggtggtaatcaattaaggctcgcagttaaaatttctcaggctttaactggtcgtggctctttttttgtattctttattcagttcgttgtttcgttatatctagtatatcgctttttaaaaaaataagcaatgatttcgtgcattattcacacgaaatcattgcttttttcttcttccatttctaactccaatgttacttgttctgtttctggttctggttctgttggctcatttgggattaaatccactactagcgttgagttagttccgtctctaatagccggttaagtaatagccggttaagtggtcaaactttgggaaaatctcaacccgcattaagttttgatgccatgacaatcgttggaaatttgaacaaaactaatgctaaaaagctatctgactttatgagtgtagagccacaaatacgactttgggatatacttcaaacaaagtttaaagctaaggcacttcaagaaaaagtttatatcgaatatgacaaagtaaaagcagatacttgggatagacgtaatatgcgtgttgaatttaatcccaataaactcacacatgaagaaatgatttggttaaaacaaaatattatcgactacatggaagatgacggttttacaagattagacttagcttttgattttgaagatgatttgagcgattactatgcaatgactgataaagcagttaagaaaactgttttttatggtcgtaatggcaagccagaaacaaaatattttggtgtccgtgatagtgatagatttattagaatttataataaaaaacaagaacgtaaagataacgcagatgttgaagttgtgtttgaacatttatggcgtgtagaagttgaattaaaaagagatatggttgattactggaatgattgttttaatgatttacacatctttgaaacctgcgtgggctactttagaaaaaattaatgagcaagctatggtttatactttgttgcatgaagaaagtatgtggggaaagctaagtaagaatactaagactaaatttaaaaaattgattagagaaatatctccaattgatttaacggaattaatgaaatcgactttaaaagcgaacgaaaaacaattgcaaaagcagattgatttttggcaacgtgaatttaggttttggaagtaaaataagttttatttgataaaaattgctaattcagtataattaatatttacgaggtgacataacgtatgaaaaaatcagaggattattcctcctaaatataaaaatttaaaatttaggaggaagttatatatgacttttaatattattgaattagaaaattgggatagaaaagaatattttgaacactattttaatcagcaaactacttatagcattactaaagaaattgatattactttgtttaaagatatgataaaaaagaaaggatatgaaatttatccctctttaatttatgcaattatggaagttgtaaataaaaataaagtgtttagaacaggaattaatagtgagaataaattaggttattgggataagttaaatcctttgtatacagtttttaataagcaaactgaaaaatttactaacatttggactgaatctgataaaaacttcatttctttttataataattataaaaatgacttgcttgaatataaagataaagaagaaatgtttcctaaaaaaccgatacctgaaaacaccataccgatttcaatgattccttggattgattttagttcatttaatttaaatattggtaacaatagcagctttttattgcctattattacgataggtaaattttatagtgagaataataaaatttatataccagttgctctgcaacttcatcattctgtatgtgatggttaccatgcttcactatttatgaatgaatttcaagatataattcatagggtagatgattggatttagtttttagattttgaaagtgaatttaattttatacacgtaagtgatcataaaatttatgaacgtataacaaccacattttttggttgcttgtggttttgattttgaatttggttttgaacttatggactgatttattcagtccattttttgtgcttgcacaaaaactagcctcgcagagcacacgcattaatgacttatgaaacgtagtaaataagtctagtgtgttatactttacttggaagatgcaccgaataaaaaatattgaagaacaactagcaaaagattttaaagagttattttattttaagtctttataacatgagtgaagcgaatttttaaatttcgatagaaatttttacatcaaaaagccccctgtcaaaattgacgaagggggttttttggcgcacgcttttcgttagaaatatacaagattgaaaatcgtgtataagtgcgccctttgttttgaacttagcacgttacatcaattttttaaaatgatgtataagtgcgcccttttaaattttgagtgattatattttttgagttagaaaaagggattgggaaaatttcccaaaataatttaaaaaataagcaaaaattttcgatagagaatgtgctattttttgtcaaaggtgtataccttgactgtgcttgctgttacattaagtttatttttaagttattaaaaaagaaatagcttttaaagtttggctcgctgtcgctttataaagctgattgacttttgattgcaaactacttaaagaaaacaaactcggactattcgttttcttctctttggtttgaacatcagcaattatcccctcttgattgcctattttagcttgtttagaagaaacaaaagctaaaagctcctcttgggttttaaaacgctgtgtggggcttagaacgcccttaaacgacccttggtttacttttatactagcttccacctcgaaaaaaggttcttttttaaaattctctatggcttcctggcgctgaaaaaataaggtataaggtgggcgtttgaacacgtcctagtgaaaatgtaccttgtacgccccttctgttgtaaatttaacgtatacaaagggcttgcgttcatgccgatcaaccaatcggcaatttggcgtgtttgcgcttcttgataaaagggatagtaattcattccaggttgcaaattttgaaaaccgcttcggattacatctttttctaagctattgatccatagtcttttaaatgttttatcttttgaaaaggcatttgctttatggataatcgaccaggcgatattttcaccttctctgtcgctatctgttgcaacaataattgtatttgcctttttgagaagttctgcaacaattttaaactgctttcccttatcttttgcaacttcaaaatcgtatcgatcaggaaaaatcggcaaagattcaagtttccaattttgccacttttcgtcataatgacctggttctgctaattccactaaatgcccaaaaccaaaggtgataaacgtttcatctgtaaatagtgggtctttgatctcaaaataaccgtcttttttggtgctttgttttaaagcacttgcgtaggctaatgcctggcttggtttttcagctaaaataaccgtactcattaactatccctcttttcattgttttttctttgatcgactgtcacgttatatcttgctcgataccttctaaacgttcggcgattgattccagtttgttcttcaacttctttatcggataaaccattcaaaaacaaatcgaaagccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgac

SEQ ID NO: 45 - DNA, 13255 bp, Streptococcus agalactiae
gtgacatgactgaaataggtagttgagatatggtatgcaatgtttgaacatgtttaaaatcgaatgtaaccatttgatagaccgccttcattatcatttctagaatttttctttaggtttgtaaagactacaaaataaaatgatgaaaacaactatcttgtggatacactaaaaagacacgctaattagcaaactctctcttcatcatctctcaccattattatactactatttatatgacaaataaaggttattttgttaaaatataactttgaaaatccacatatatttttaatcttccgtctgaaaaaataaataaaaatagtaaaaataaacacgaatttaaaataagcaaattttttaagaaaatctgtgctaaactttaatagttttgtgcttaataataatcagcacttacaaagaacaaagggaagcggccgcgacatatttgctctgatatggcaggaggtaaggaaggaaaatgatacctaaagttatacattattgttggtttggaggaaatcccttaccagataatttaaagaaatatataaaaacttggagagaacaatgtccggattatgaaattattgaatggaatgagcataattatgatgttagtaaaaatgtttttatgagagaagcatatactaagaagaattttgcttatgtttctgactatgcaagattggatattatttatacttatggggggttctatctagatactgatgtggagcttttaaaaagtttagatcctttgaggattcatgagtgttttctagcaagggagattagttgtgatgttgccactggattaattattggtgcagttagaggacaaaaatttataaaagataatatgatggtatatgaaaaatataaatttttaaattttgataaaacttgtgtggaaattacgactgacttacttttaaaaaaaggattgaagaataaagatgtcatacaagttatagatggtgttactatttatccaagacaatattttaatcctaaaagtatgttgacggggaggttagattttctaacagatgatacatactctatacaccattatgaaggaagctggaaaaaaagatcatttgttcctgattccctaaagattagattacgtcttataatcgattttgtgttcggatatggaacatatagaaggatattgcgttttttaaaattaaaatagtagcagttattaatttaaaatattctctttaaaaaatggaggcacaatgaaagtatcattagttataccagtttataatggagcaaatcatatcaaagaaacattagaaagtattatagcgcagacctaccccaatttggaaatattaatcattgatgatgggtcaactgataatacatcaactatatgtcagaagtatgtttctaaagataagcgcatgaaatatatatataaaaaaaatggaggtcaatcttctgctagaaatttaggcgttaaactatctacaagtaattggatagctttcttagattctgatgatctttgttcgccattttatatagaatgtctagtgagaattcaacatgaaactggtgctgaccttgtaataactggaattgaggatcacccttgttacagaacggcaaaactttcatttgataaactatcttactcagtactcagtgtggaagagacactaatagaaatgtattatgataactatggtatttctccattagggaagttatattccaaagggttattattaaaatatccataccctattggtaaaattcatgaagatacagacacaacttataaactgattgcaagtagtacacgaattgcattgagcaaagagaaaacagtatatgtcaaagggcaacctaatagtactaccagacagtcatacaaagagaatatgttatattattttgaagcaatgcaacataatagagattttatccttgaacattattcagataaagaactaattgatgctttaaattataaaactgtaattggaggaatgtggatcgttaataagcttttagaaggagataagtacgattatattaattctttgagaagtgaatatagaaaatattataagagtatattatttaataagcgagttacaattaaagaaaaaataaaatatttttcatttatttcgtcaatgaatttatttttatttttaaaaaaattttataactataatgggagataaattatgaagatgattagtatcattatcccagatctataaaaggctacttcttaagtaaccatgtttaagaaaagagaaatagccttatttctctttttgtcgtttttaaaataaaggaacatcatgaaacaaacattaaaacttatgttttcttttctgttgatgttagggactatgtttggaattagccaaactgttttagcgcaagaaactcatcagttgacgattgttcatcttgaagcaagggatattgatcgtccaaatccacagttggagattgcccctaaagaagggactccaattgaaggagtactctatcagttgtaccaattaaaatcaactgaagatggcgatgtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgactttaaacgtggatcattttctttaaatttatgctgacgacctttgaatttgccttttttcttagcaatttcgattccttgtgcctgacgttccttaattttttttcgttctgattctgcttgatacttgtacaattcaatgacaaggctattaatcaaacgccttaaattttcatcttcaataccattcattgagggtaaatttaagacttccagggttgcccccttaatttgaatttgattcatcaattctgttaattctttattatttcgtcctaatcgatctaattcagtaacaataacaatatccccttcacgaatatagttaagcatagcttgtaattgtgggcgttcgaccgattgaccgcttaatttgtctgaaaagaccttagaaacgccctgtaacgcttgtaattgccgatctaagttctgttctttgctactgacacgtgcataaccaattttagccattttcaaccaacctctaaaattctctcggttgcaataaccaatcagcaatatctactttttcaatttcaaattgcttatcagaaattgtcttttcgtaagcgataaaatcttgcgcatattgttgctcattaaaaatagccaccacttcgtcattttctaaaactcgataaataaattttttcattttactcctcctattatgcccaacttaaatgacctattcaccaagtcaattatactgctaaaatcatattaggacaaataggtatactctattgacctataaatgatagcaacttaaaagatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcactaaacagcaaagaaaaacaaacacgtatcatagatataaatgtaatggcatagtgcgggttttattttcagcctgtatcgtagctaaacaaatcgagttgtgggtccgttttggggcgttctgccaatttgtttagagtttcttgaataaatgtacgttctaaattaaacgaagctgtcagcgcctttatatagctttctcgttcttctttttttaatttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcgctaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacataacgctattgcgccctactaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattgggatcaaaaaattctacattgtccgttcttggtatacgagcaatcccaaaatgattgcacgttagatcaactggcaaagactttccaaaatattctcggatattttgcgagattattttggctgctttgacagatttaaattctgattttgaagtcacatagactggcgtttctaaaacaaaatatgcttgataacctttatcagatttgataattaacgtaggcataaaacctaaatcaatagctgttgttaaaatatcgcttgctgaaatagtttctttttccgtgtgaatatcaaaatcaataaagaaggtattgatttgtcttaaattgttttcagaatgtcctttagtgtatgaacggttttcgtctgcatacgtaccataacgataaacgtttggtgtccaatgcgtaaatgtatcttgattttcgtgaatcgcttcttcggaagtcagaacaacgccacgtccgccaatcatgcttttttttgagcgatacgcaaaaatagcccctttacttttacctggcttggtagtgattgagcgaattttactatttttaaatttgtactttaacaagccgtcatgaagcacagtttctacaacaaaagggatattcattcagctgttctcctttcttacgaaaattaattagttagaagctacgatcaaagttgaatcacaacaaaaaaggcaatcaactaagtttttcttaattgattgcctggtatcttcttaaagacttgaaatcccctcaaaaacccgatataatgggtttacagatatttaagtatctgattaataaagtaattaaatactttaccaaattttgggtctcgacttctttaattgattggtggtaatcaattaaggctcgcagttaaaatttctcaggctttaactggtcgtggctctttttttgtattctttattcagttcgttgtttcgttatatctagtatatcgctttttaaaaaaataagcaatgatttcgtgcattattcacacgaaatcattgcttttttcttcttccatttctaactccaatgttacttgttctgtttctggttctggttctgttggctcatttgggattaaatccactactagcgttgagttagttccgtctctaatagccggttaagtaatagccggttaagtggtcaaactttgggaaaatctcaacccgcattaagttttgatgccatgacaatcgttggaaatttgaacaaaactaatgctaaaaagctatctgactttatgagtgtagagccacaaatacgactttgggatatacttcaaacaaagtttaaagctaaggcacttcaagaaaaagtttatatcgaatatgacaaagtaaaagcagatacttgggatagacgtaatatgcgtgttgaatttaatcccaataaactcacacatgaagaaatgatttggttaaaacaaaatattatcgactacatggaagatgacggttttacaagattagacttagcttttgattttgaagatgatttgagcgattactatgcaatgactgataaagcagttaagaaaactgttttttatggtcgtaatggcaagccagaaacaaaatattttggtgtccgtgatagtgatagatttattagaatttataataaaaaacaagaacgtaaagataacgcagatgttgaagttgtgtttgaacatttatggcgtgtagaagttgaattaaaaagagatatggttgattactggaatgattgttttaatgatttacacatctttgaaacctgcgtgggctactttagaaaaaattaatgagcaagctatggtttatactttgttgcatgaagaaagtatgtggggaaagctaagtaagaatactaagactaaatttaaaaaattgattagagaaatatctccaattgatttaacggaattaatgaaatcgactttaaaagcgaacgaaaaacaattgcaaaagcagattgatttttggcaacgtgaatttaggttttggaagtaaaataagttttatttgataaaaattgctaattcagtataattaatatttacgaggtgacataacgtatgaaaaaatcagaggattattcctcctaaatataaaaatttaaaatttaggaggaagttatatatgacttttaatattattgaattagaaaattgggatagaaaagaatattttgaacactattttaatcagcaaactacttatagcattactaaagaaattgatattactttgtttaaagatatgataaaaaagaaaggatatgaaatttatccctctttaatttatgcaattatggaagttgtaaataaaaataaagtgtttagaacaggaattaatagtgagaataaattaggttattgggataagttaaatcctttgtatacagtttttaataagcaaactgaaaaatttactaacatttggactgaatctgataaaaacttcatttctttttataataattataaaaatgacttgcttgaatataaagataaagaagaaatgtttcctaaaaaaccgatacctgaaaacaccataccgatttcaatgattccttggattgattttagttcatttaatttaaatattggtaacaatagcagctttttattgcctattattacgataggtaaattttatagtgagaataataaaatttatataccagttgctctgcaacttcatcattctgtatgtgatggttaccatgcttcactatttatgaatgaatttcaagatataattcatagggtagatgattggatttagtttttagattttgaaagtgaatttaattttatacacgtaagtgatcataaaatttatgaacgtataacaaccacattttttggttgcttgtggttttgattttgaatttggttttgaacttatggactgatttattcagtccattttttgtgcttgcacaaaaactagcctcgcagagcacacgcattaatgacttatgaaacgtagtaaataagtctagtgtgttatactttacttggaagatgcaccgaataaaaaatattgaagaacaactagcaaaagattttaaagagttattttattttaagtctttataacatgagtgaagcgaatttttaaatttcgatagaaatttttacatcaaaaagccccctgtcaaaattgacgaagggggttttttggcgcacgcttttcgttagaaatatacaagattgaaaatcgtgtataagtgcgccctttgttttgaacttagcacgttacatcaattttttaaaatgatgtataagtgcgcccttttaaattttgagtgattatattttttgagttagaaaaagggattgggaaaatttcccaaaataatttaaaaaataagcaaaaattttcgatagagaatgtgctattttttgtcaaaggtgtataccttgactgtgcttgctgttacattaagtttatttttaagttattaaaaaagaaatagcttttaaagtttggctcgctgtcgctttataaagctgattgacttttgattgcaaactacttaaagaaaacaaactcggactattcgttttcttctctttggtttgaacatcagcaattatcccctcttgattgcctattttagcttgtttagaagaaacaaaagctaaaagctcctcttgggttttaaaacgctgtgtggggcttagaacgcccttaaacgacccttggtttacttttatactagcttccacctcgaaaaaaggttcttttttaaaattctctatggcttcctggcgctgaaaaaataaggtataaggtgggcgtttgaacacgtcctagtgaaaatgtaccttgtacgccccttctgttgtaaatttaacgtatacaaagggcttgcgttcatgccgatcaaccaatcggcaatttggcgtgtttgcgcttcttgataaaagggatagtaattcattccaggttgcaaattttgaaaaccgcttcggattacatctttttctaagctattgatccatagtcttttaaatgttttatcttttgaaaaggcatttgctttatggataatcgaccaggcgatattttcaccttctctgtcgctatctgttgcaacaataattgtatttgcctttttgagaagttctgcaacaattttaaactgctttcccttatcttttgcaacttcaaaatcgtatcgatcaggaaaaatcggcaaagattcaagtttccaattttgccacttttcgtcataatgacctggttctgctaattccactaaatgcccaaaaccaaaggtgataaacgtttcatctgtaaatagtgggtctttgatctcaaaataaccgtcttttttggtgctttgttttaaagcacttgcgtaggctaatgcctggcttggtttttcagctaaaataaccgtactcattaactatccctcttttcattgttttttctttgatcgactgtcacgttatatcttgctcgataccttctaaacgttcggcgattgattccagtttgttcttcaacttctttatcggataaaccattcaaaaacaaatcgaaagccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgac

SEQ ID NO: 46 - DNA, 13817 bp, Streptococcus agalactiae
ttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgactttaaacgtggatcattttctttaaatttatgctgacgacctttgaatttgccttttttcttagcaatttcgattccttgtgcctgacgttccttaattttttttcgttctgattctgcttgatacttgtacaattcaatgacaaggctattaatcaaacgccttaaattttcatcttcaataccattcattgagggtaaatttaagacttccagggttgcccccttaatttgaatttgattcatcaattctgttaattctttattatttcgtcctaatcgatctaattcagtaacaataacaatatccccttcacgaatatagttaagcatagcttgtaattgtgggcgttcgaccgattgaccgcttaatttgtctgaaaagaccttagaaacgccctgtaacgcttgtaattgccgatctaagttctgttctttgctactgacacgtgcataaccaattttagccattttcaaccaacctctaaaattctctcggttgcaataaccaatcagcaatatctactttttcaatttcaaattgcttatcagaaattgtcttttcgtaagcgataaaatcttgcgcatattgttgctcattaaaaatagccaccacttcgtcattttctaaaactcgataaataaattttttcattttactcctcctattatgcccaacttaaatgacctattcaccaagtcaattatactgctaaaatcatattaggacaaataggtatactctattgacctataaatgatagcaacttaaaagatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcactaaacagcaaagaaaaacaaacacgtatcatagatataaatgtaatggcatagtgcgggttttattttcagcctgtatcgtagctaaacaaatcgagttgtgggtccgttttggggcgttctgccaatttgtttagagtttcttgaataaatgtacgttctaaattaaacgaagctgtcagcgcctttatatagctttctcgttcttctttttttaatttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcgctaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacataacgctattgcgccctactaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattgggatcaaaaaattctacattgtccgttcttggtatacgagcaatcccaaaatgattgcacgttagatcaactggcaaagactttccaaaatattctcggatattttgcgagattattttggctgctttgacagatttaaattctgattttgaagtcacatagactggcgtttctaaaacaaaatatgcttgataacctttatcagatttgataattaacgtaggcataaaacctaaatcaatagctgttgttaaaatatcgcttgctgaaatagtttctttttccgtgtgaatatcaaaatcaataaagaaggtattgatttgtcttaaattgttttcagaatgtcctttagtgtatgaacggttttcgtctgcatacgtaccataacgataaacgtttggtgtccaatgcgtaaatgtatcttgattttcgtgaatcgcttcttcggaagtcagaacaacgccacgtccgccaatcatgcttttttttgagcgatacgcaaaaatagcccctttacttttacctggcttggtagtgattgagcgaattttactatttttaaatttgtactttaacaagccgtcatgaagcacagtttctacaacaaaagggatattcattcagctgttctcctttcttacgaaaattaattagttagaagctacgatcaaagttgaatcacaacaaaaaaggcaatcaactaagtttttcttaattgattgcctggtatcttcttaaagacttgaaatcccctcaaaaacccgatataatgggtttacagatatttaagtatctgattaataaagtaattaaatactttaccaaattttgggtctcgacttctttaattgattggtggtaatcaattaaggctcgcagttaaaatttctcaggctttaactggtcgtggctctttttttgtattctttattcagttcgttgtttcgttatatctagtatatcgctttttaaaaaaataagcaatgatttcgtgcattattcacacgaaatcattgcttttttcttcttccatttctaactccaatgttacttgttctgtttctggttctggttctgttggctcatttgggattaaatccactactagcgttgagttagttccgtctctaatagccggttaagtaatagccggttaagtggtcaaactttgggaaaatctcaacccgcattaagttttgatgccatgacaatcgttggaaatttgaacaaaactaatgctaaaaagctatctgactttatgagtgtagagccacaaatacgactttgggatatacttcaaacaaagtttaaagctaaggcacttcaagaaaaagtttatatcgaatatgacaaagtaaaagcagatacttgggatagacgtaatatgcgtgttgaatttaatcccaataaactcacacatgaagaaatgatttggttaaaacaaaatattatcgactacatggaagatgacggttttacaagattagacttagcttttgattttgaagatgatttgagcgattactatgcaatgactgataaagcagttaagaaaactgttttttatggtcgtaatggcaagccagaaacaaaatattttggtgtccgtgatagtgatagatttattagaatttataataaaaaacaagaacgtaaagataacgcagatgttgaagttgtgtttgaacatttatggcgtgtagaagttgaattaaaaagagatatggttgattactggaatgattgttttaatgatttacacatctttgaaacctgcgtgggctactttagaaaaaattaatgagcaagctatggtttatactttgttgcatgaagaaagtatgtggggaaagctaagtaagaatactaagactaaatttaaaaaattgattagagaaatatctccaattgatttaacggaattaatgaaatcgactttaaaagcgaacgaaaaacaattgcaaaagcagattgatttttggcaacgtgaatttaggttttggaagtaaaataagttttatttgataaaaattgctaattcagtataattaatatttacgaggtgacataacgtatgaaaaaatcagaggattattcctcctaaatataaaaatttaaaatttaggaggaagttatatatgacttttaatattattgaattagaaaattgggatagaaaagaatattttgaacactattttaatcagcaaactacttatagcattactaaagaaattgatattactttgtttaaagatatgataaaaaagaaaggatatgaaatttatccctctttaatttatgcaattatggaagttgtaaataaaaataaagtgtttagaacaggaattaatagtgagaataaattaggttattgggataagttaaatcctttgtatacagtttttaataagcaaactgaaaaatttactaacatttggactgaatctgataaaaacttcatttctttttataataattataaaaatgacttgcttgaatataaagataaagaagaaatgtttcctaaaaaaccgatacctgaaaacaccataccgatttcaatgattccttggattgattttagttcatttaatttaaatattggtaacaatagcagctttttattgcctattattacgataggtaaattttatagtgagaataataaaatttatataccagttgctctgcaacttcatcattctgtatgtgatggttaccatgcttcactatttatgaatgaatttcaagatataattcatagggtagatgattggatttagtttttagattttgaaagtgaatttaattttatacacgtaagtgatcataaaatttatgaacgtataacaaccacattttttggttgcttgtggttttgattttgaatttggttttgaacttatggactgatttattcagtccattttttgtgcttgcacaaaaactagcctcgcagagcacacgcattaatgacttatgaaacgtagtaaataagtctagtgtgttatactttacttggaagatgcaccgaataaaaaatattgaagaacaactagcaaaagattttaaagagttattttattttaagtctttataacatgagtgaagcgaatttttaaatttcgatagaaatttttacatcaaaaagccccctgtcaaaattgacgaagggggttttttggcgcacgcttttcgttagaaatatacaagattgaaaatcgtgtataagtgcgccctttgttttgaacttagcacgttacatcaattttttaaaatgatgtataagtgcgcccttttaaattttgagtgattatattttttgagttagaaaaagggattgggaaaatttcccaaaataatttaaaaaataagcaaaaattttcgatagagaatgtgctattttttgtcaaaggtgtataccttgactgtgcttgctgttacattaagtttatttttaagttattaaaaaagaaatagcttttaaagtttggctcgctgtcgctttataaagctgattgacttttgattgcaaactacttaaagaaaacaaactcggactattcgttttcttctctttggtttgaacatcagcaattatcccctcttgattgcctattttagcttgtttagaagaaacaaaagctaaaagctcctcttgggttttaaaacgctgtgtggggcttagaacgcccttaaacgacccttggtttacttttatactagcttccacctcgaaaaaaggttcttttttaaaattctctatggcttcctggcgctgaaaaaataaggtataaggtgggcgtttgaacacgtcctagtgaaaatgtaccttgtacgccccttctgttgtaaatttaacgtatacaaagggcttgcgttcatgccgatcaaccaatcggcaatttggcgtgtttgcgcttcttgataaaagggatagtaattcattccaggttgcaaattttgaaaaccgcttcggattacatctttttctaagctattgatccatagtcttttaaatgttttatcttttgaaaaggcatttgctttatggataatcgaccaggcgatattttcaccttctctgtcgctatctgttgcaacaataattgtatttgcctttttgagaagttctgcaacaattttaaactgctttcccttatcttttgcaacttcaaaatcgtatcgatcaggaaaaatcggcaaagattcaagtttccaattttgccacttttcgtcataatgacctggttctgctaattccactaaatgcccaaaaccaaaggtgataaacgtttcatctgtaaatagtgggtctttgatctcaaaataaccgtcttttttggtgctttgttttaaagcacttgcgtaggctaatgcctggcttggtttttcagctaaaataaccgtactcattaactatccctcttttcattgttttttctttgatcgactgtcacgttatatcttgctcgataccttctaaacgttcggcgattgattccagtttgttcttcaacttctttatcggataaaccattcaaaaacaaatcgaaagccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgtgacatgactgaaataggtagttgagatatggtatgcaatgtttgaacatgtttaaaatcgaatgtaaccatttgatagaccgccttcattatcatttctagaatttttctttaggtttgtaaagactacaaaataaaatgatgaaaacaactatcttgtggatacactaaaaagacacgctaattagcaaactctctcttcatcatctctcaccattattatactactatttatatgacaaataaaggttattttgttaaaatataactttgaaaatccacatatatttttaatcttccgtctgaaaaaataaataaaaatagtaaaaataaacacgaatttaaaataagcaaattttttaagaaaatctgtgctaaactttaatagttttgtgcttaataataatcagcacttacaaagaacaaagggaagcggccgcaattataggtgaaatatgaggaaatatctagatttagattattctttattttatgctctttgggtacttattttagtaccaaaccaatggtatcagtttttaattattaccattatagttctattattactttggaagagtgagtttagaatatctataagcaattcttcaatactatttctgctttggttatttatttatttatttgcaatactcattagaggtactcaagaggatataacgtttcagcgatttattgctgagctattaaaactaattagtacaggatatgctttatttttttataattattatagaaaagctgattttaatagttcagttgtaaggaatgtggtaaaggttaactattttgtgttgtttcttataacagttttatatttattttttcctatgctgaagccaactttatttggaagagaattgttttcaatagagtggtttccacatatgagaataagacttgcggcatattttgaatatgctacactaattggtcagtttattttattttcttatcccatactttttttgaaaccccaaaaacatatggaaaatattttaatatccttactgttgactatatgttcatacttttctggcgctagaatactattggtctgtatgttggttttattagcatcgcttcttttagattatatcctttttaaaactaatttgaaattgaccaagaaaaacacttttatacttggtatgactttcttatttatcaccgcttgtttttcttataacatatggtcaataattgaaaaaataattatgtacagaaaccaaagtactatcactaggatgatagtttatcaagaaagtattattgaagttctaaaaggaaatattttatttggacagggtataaggattccatcaagtgaaggaatattcctaggatcgcattctacttatattagtgtcttttacaggacttctttattaggaattgttctttatttttctgcctttatacttttatataaagaagcgatttcaaaaaattataaaatctacagattatttttttatacgttattatgttacacgctctttgaggaaatagatcctaatcattggagtattgtattattattctcaacttttggtatagtgggaagggctaaaaaatgagttaataggaggaattttcgctttaaccctattttcaaagccaatgcaacttttgttacttttagcattaatagttttacttatttgtagtagttataatgaaaaaatgaaatttttaaatatggctgaaatttttttcattgtattttatattatttatttaacttcaatattgctacattctttgtttaaaaatcctgattttgatagaattttagcagcttttaactcgttgattatcggtatagtatcattggctttttatcattattataagaatacaactttggagttagataaaatattaaaagcatttttatttaatgggttaatcctattttttttagggggaacatattattattgtttgcataataatattcaaaatatcagtatttttggtagagatttgattgggtcagactggattaatggtatgcatactcaaagagcaatgggattttttgaatattcaaaccttataattcctattacagtggtaactaacttatatatatatatatatatgaagttaagaaactattcaattatgaccataggtgttgtattattatttacctttattttacctattggatcgggctccagggctggaatagtagctatattggcgcagatgtttattcttcttctaaatacagttgtcgtaaagaagaaaactataaaatttttattgtacatacttccgtttctactagtaatagtaatgatgttatattttgataacttactatctatatattatcgtataattaatttgcgatccgggagtagtgaatccagattttctttatataaagatacagtaaacatcgttataaataattctttattatttggagaaggagttaaagagttatggttaaatagtgatctacctttggggtcgcattcaacgtatataggctatttctacaaaagtggcctgctgggattaatgaatatagttccaggtttgcttttaatttttactaatattggtaggaaagctaaacaatcagctttttattatgagatagtaggaacacttataactttattctcattttttgcacttgaagatcttgacggagctaattggcttattgtttttatttttacagtgttaggaattttagaaaataaggatttttatagtcaacttaaaaggtggaaaagttaagcggccgcatcgatcgatgagatctataaaaggctacttcttaagtaaccatgtttaagaaaagagaaatagccttatttctctttttgtcgtttttaaaataaaggaacatcatgaaacaaacattaaaacttatgttttcttttctgttgatgttagggactatgtttggaattagccaaactgttttagcgcaagaaactcatcagttgacgattgttcatcttgaagcaagggatattgatcgtccaaatccacagttggagattgcccctaaagaagggactccaattgaaggagtactctatcagttgtaccaattaaaatcaactgaagatggcgatgtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttc

SEQ ID NO: 47 - DNA, 2377 bp, Streptococcus agalactiae
gcggccgcaattataggtgaaatatgaggaaatatctagatttagattattctttattttatgctctttgggtacttattttagtaccaaaccaatggtatcagtttttaattattaccattatagttctattattactttggaagagtgagtttagaatatctataagcaattcttcaatactatttctgctttggttatttatttatttatttgcaatactcattagaggtactcaagaggatataacgtttcagcgatttattgctgagctattaaaactaattagtacaggatatgctttatttttttataattattatagaaaagctgattttaatagttcagttgtaaggaatgtggtaaaggttaactattttgtgttgtttcttataacagttttatatttattttttcctatgctgaagccaactttatttggaagagaattgttttcaatagagtggtttccacatatgagaataagacttgcggcatattttgaatatgctacactaattggtcagtttattttattttcttatcccatactttttttgaaaccccaaaaacatatggaaaatattttaatatccttactgttgactatatgttcatacttttctggcgctagaatactattggtctgtatgttggttttattagcatcgcttcttttagattatatcctttttaaaactaatttgaaattgaccaagaaaaacacttttatacttggtatgactttcttatttatcaccgcttgtttttcttataacatatggtcaataattgaaaaaataattatgtacagaaaccaaagtactatcactaggatgatagtttatcaagaaagtattattgaagttctaaaaggaaatattttatttggacagggtataaggattccatcaagtgaaggaatattcctaggatcgcattctacttatattagtgtcttttacaggacttctttattaggaattgttctttatttttctgcctttatacttttatataaagaagcgatttcaaaaaattataaaatctacagattatttttttatacgttattatgttacacgctctttgaggaaatagatcctaatcattggagtattgtattattattctcaacttttggtatagtgggaagggctaaaaaatgagttaataggaggaattttcgctttaaccctattttcaaagccaatgcaacttttgttacttttagcattaatagttttacttatttgtagtagttataatgaaaaaatgaaatttttaaatatggctgaaatttttttcattgtattttatattatttatttaacttcaatattgctacattctttgtttaaaaatcctgattttgatagaattttagcagcttttaactcgttgattatcggtatagtatcattggctttttatcattattataagaatacaactttggagttagataaaatattaaaagcatttttatttaatgggttaatcctattttttttagggggaacatattattattgtttgcataataatattcaaaatatcagtatttttggtagagatttgattgggtcagactggattaatggtatgcatactcaaagagcaatgggattttttgaatattcaaaccttataattcctattacagtggtaactaacttatatatatatatatatatgaagttaagaaactattcaattatgaccataggtgttgtattattatttacctttattttacctattggatcgggctccagggctggaatagtagctatattggcgcagatgtttattcttcttctaaatacagttgtcgtaaagaagaaaactataaaatttttattgtacatacttccgtttctactagtaatagtaatgatgttatattttgataacttactatctatatattatcgtataattaatttgcgatccgggagtagtgaatccagattttctttatataaagatacagtaaacatcgttataaataattctttattatttggagaaggagttaaagagttatggttaaatagtgatctacctttggggtcgcattcaacgtatataggctatttctacaaaagtggcctgctgggattaatgaatatagttccaggtttgcttttaatttttactaatattggtaggaaagctaaacaatcagctttttattatgagatagtaggaacacttataactttattctcattttttgcacttgaagatcttgacggagctaattggcttattgtttttatttttacagtgttaggaattttagaaaataaggatttttatagtcaacttaaaaggtggaaaagttaagcggccgcatcgatcgatgagatct

SEQ ID NO: 48 - DNA, 14542 bp, Streptococcus agalactiae
ttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgactttaaacgtggatcattttctttaaatttatgctgacgacctttgaatttgccttttttcttagcaatttcgattccttgtgcctgacgttccttaattttttttcgttctgattctgcttgatacttgtacaattcaatgacaaggctattaatcaaacgccttaaattttcatcttcaataccattcattgagggtaaatttaagacttccagggttgcccccttaatttgaatttgattcatcaattctgttaattctttattatttcgtcctaatcgatctaattcagtaacaataacaatatccccttcacgaatatagttaagcatagcttgtaattgtgggcgttcgaccgattgaccgcttaatttgtctgaaaagaccttagaaacgccctgtaacgcttgtaattgccgatctaagttctgttctttgctactgacacgtgcataaccaattttagccattttcaaccaacctctaaaattctctcggttgcaataaccaatcagcaatatctactttttcaatttcaaattgcttatcagaaattgtcttttcgtaagcgataaaatcttgcgcatattgttgctcattaaaaatagccaccacttcgtcattttctaaaactcgataaataaattttttcattttactcctcctattatgcccaacttaaatgacctattcaccaagtcaattatactgctaaaatcatattaggacaaataggtatactctattgacctataaatgatagcaacttaaaagatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcactaaacagcaaagaaaaacaaacacgtatcatagatataaatgtaatggcatagtgcgggttttattttcagcctgtatcgtagctaaacaaatcgagttgtgggtccgttttggggcgttctgccaatttgtttagagtttcttgaataaatgtacgttctaaattaaacgaagctgtcagcgcctttatatagctttctcgttcttctttttttaatttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcgctaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacataacgctattgcgccctactaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattgggatcaaaaaattctacattgtccgttcttggtatacgagcaatcccaaaatgattgcacgttagatcaactggcaaagactttccaaaatattctcggatattttgcgagattattttggctgctttgacagatttaaattctgattttgaagtcacatagactggcgtttctaaaacaaaatatgcttgataacctttatcagatttgataattaacgtaggcataaaacctaaatcaatagctgttgttaaaatatcgcttgctgaaatagtttctttttccgtgtgaatatcaaaatcaataaagaaggtattgatttgtcttaaattgttttcagaatgtcctttagtgtatgaacggttttcgtctgcatacgtaccataacgataaacgtttggtgtccaatgcgtaaatgtatcttgattttcgtgaatcgcttcttcggaagtcagaacaacgccacgtccgccaatcatgcttttttttgagcgatacgcaaaaatagcccctttacttttacctggcttggtagtgattgagcgaattttactatttttaaatttgtactttaacaagccgtcatgaagcacagtttctacaacaaaagggatattcattcagctgttctcctttcttacgaaaattaattagttagaagctacgatcaaagttgaatcacaacaaaaaaggcaatcaactaagtttttcttaattgattgcctggtatcttcttaaagacttgaaatcccctcaaaaacccgatataatgggtttacagatatttaagtatctgattaataaagtaattaaatactttaccaaattttgggtctcgacttctttaattgattggtggtaatcaattaaggctcgcagttaaaatttctcaggctttaactggtcgtggctctttttttgtattctttattcagttcgttgtttcgttatatctagtatatcgctttttaaaaaaataagcaatgatttcgtgcattattcacacgaaatcattgcttttttcttcttccatttctaactccaatgttacttgttctgtttctggttctggttctgttggctcatttgggattaaatccactactagcgttgagttagttccgtctctaatagccggttaagtaatagccggttaagtggtcaaactttgggaaaatctcaacccgcattaagttttgatgccatgacaatcgttggaaatttgaacaaaactaatgctaaaaagctatctgactttatgagtgtagagccacaaatacgactttgggatatacttcaaacaaagtttaaagctaaggcacttcaagaaaaagtttatatcgaatatgacaaagtaaaagcagatacttgggatagacgtaatatgcgtgttgaatttaatcccaataaactcacacatgaagaaatgatttggttaaaacaaaatattatcgactacatggaagatgacggttttacaagattagacttagcttttgattttgaagatgatttgagcgattactatgcaatgactgataaagcagttaagaaaactgttttttatggtcgtaatggcaagccagaaacaaaatattttggtgtccgtgatagtgatagatttattagaatttataataaaaaacaagaacgtaaagataacgcagatgttgaagttgtgtttgaacatttatggcgtgtagaagttgaattaaaaagagatatggttgattactggaatgattgttttaatgatttacacatctttgaaacctgcgtgggctactttagaaaaaattaatgagcaagctatggtttatactttgttgcatgaagaaagtatgtggggaaagctaagtaagaatactaagactaaatttaaaaaattgattagagaaatatctccaattgatttaacggaattaatgaaatcgactttaaaagcgaacgaaaaacaattgcaaaagcagattgatttttggcaacgtgaatttaggttttggaagtaaaataagttttatttgataaaaattgctaattcagtataattaatatttacgaggtgacataacgtatgaaaaaatcagaggattattcctcctaaatataaaaatttaaaatttaggaggaagttatatatgacttttaatattattgaattagaaaattgggatagaaaagaatattttgaacactattttaatcagcaaactacttatagcattactaaagaaattgatattactttgtttaaagatatgataaaaaagaaaggatatgaaatttatccctctttaatttatgcaattatggaagttgtaaataaaaataaagtgtttagaacaggaattaatagtgagaataaattaggttattgggataagttaaatcctttgtatacagtttttaataagcaaactgaaaaatttactaacatttggactgaatctgataaaaacttcatttctttttataataattataaaaatgacttgcttgaatataaagataaagaagaaatgtttcctaaaaaaccgatacctgaaaacaccataccgatttcaatgattccttggattgattttagttcatttaatttaaatattggtaacaatagcagctttttattgcctattattacgataggtaaattttatagtgagaataataaaatttatataccagttgctctgcaacttcatcattctgtatgtgatggttaccatgcttcactatttatgaatgaatttcaagatataattcatagggtagatgattggatttagtttttagattttgaaagtgaatttaattttatacacgtaagtgatcataaaatttatgaacgtataacaaccacattttttggttgcttgtggttttgattttgaatttggttttgaacttatggactgatttattcagtccattttttgtgcttgcacaaaaactagcctcgcagagcacacgcattaatgacttatgaaacgtagtaaataagtctagtgtgttatactttacttggaagatgcaccgaataaaaaatattgaagaacaactagcaaaagattttaaagagttattttattttaagtctttataacatgagtgaagcgaatttttaaatttcgatagaaatttttacatcaaaaagccccctgtcaaaattgacgaagggggttttttggcgcacgcttttcgttagaaatatacaagattgaaaatcgtgtataagtgcgccctttgttttgaacttagcacgttacatcaattttttaaaatgatgtataagtgcgcccttttaaattttgagtgattatattttttgagttagaaaaagggattgggaaaatttcccaaaataatttaaaaaataagcaaaaattttcgatagagaatgtgctattttttgtcaaaggtgtataccttgactgtgcttgctgttacattaagtttatttttaagttattaaaaaagaaatagcttttaaagtttggctcgctgtcgctttataaagctgattgacttttgattgcaaactacttaaagaaaacaaactcggactattcgttttcttctctttggtttgaacatcagcaattatcccctcttgattgcctattttagcttgtttagaagaaacaaaagctaaaagctcctcttgggttttaaaacgctgtgtggggcttagaacgcccttaaacgacccttggtttacttttatactagcttccacctcgaaaaaaggttcttttttaaaattctctatggcttcctggcgctgaaaaaataaggtataaggtgggcgtttgaacacgtcctagtgaaaatgtaccttgtacgccccttctgttgtaaatttaacgtatacaaagggcttgcgttcatgccgatcaaccaatcggcaatttggcgtgtttgcgcttcttgataaaagggatagtaattcattccaggttgcaaattttgaaaaccgcttcggattacatctttttctaagctattgatccatagtcttttaaatgttttatcttttgaaaaggcatttgctttatggataatcgaccaggcgatattttcaccttctctgtcgctatctgttgcaacaataattgtatttgcctttttgagaagttctgcaacaattttaaactgctttcccttatcttttgcaacttcaaaatcgtatcgatcaggaaaaatcggcaaagattcaagtttccaattttgccacttttcgtcataatgacctggttctgctaattccactaaatgcccaaaaccaaaggtgataaacgtttcatctgtaaatagtgggtctttgatctcaaaataaccgtcttttttggtgctttgttttaaagcacttgcgtaggctaatgcctggcttggtttttcagctaaaataaccgtactcattaactatccctcttttcattgttttttctttgatcgactgtcacgttatatcttgctcgataccttctaaacgttcggcgattgattccagtttgttcttcaacttctttatcggataaaccattcaaaaacaaatcgaaagccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgtgacatgactgaaataggtagttgagatatggtatgcaatgtttgaacatgtttaaaatcgaatgtaaccatttgatagaccgccttcattatcatttctagaatttttctttaggtttgtaaagactacaaaataaaatgatgaaaacaactatcttgtggatacactaaaaagacacgctaattagcaaactctctcttcatcatctctcaccattattatactactatttatatgacaaataaaggttattttgttaaaatataactttgaaaatccacatatatttttaatcttccgtctgaaaaaataaataaaaatagtaaaaataaacacgaatttaaaataagcaaattttttaagaaaatctgtgctaaactttaatagttttgtgcttaataataatcagcacttacaaagaacaaagggaagcggccgcattataggtgaaatatgaggaaatatctagatttagattattctttattttatgctctttgggtacttattttagtaccaaaccaatggtatcagtttttaattattaccattatagttctattattactttggaagagtgagtttagaatatctataagcaattcttcaatactatttctgctttggttatttatttatttatttgcaatactcattagaggtactcaagaggatataacgtttcagcgatttattgctgagctattaaaactaattagtacaggatatgctttatttttttataattattatagaaaagctgattttaatagttcagttgtaaggaatgtggtaaaggttaactattttgtgttgtttcttataacagttttatatttattttttcctatgctgaagccaactttatttggaagagaattgttttcaatagagtggtttccacatatgagaataagacttgcggcatattttgaatatgctacactaattggtcagtttattttattttcttatcccatactttttttgaaaccccaaaaacatatggaaaatattttaatatccttactgttgactatatgttcatacttttctggcgctagaatactattggtctgtatgttggttttattagcatcgcttcttttagattatatcctttttaaaactaatttgaaattgaccaagaaaaacacttttatacttggtatgactttcttatttatcaccgcttgtttttcttataacatatggtcaataattgaaaaaataattatgtacagaaaccaaagtactatcactaggatgatagtttatcaagaaagtattattgaagttctaaaaggaaatattttatttggacagggtataaggattccatcaagtgaaggaatattcctaggatcgcattctacttatattagtgtcttttacaggacttctttattaggaattgttctttatttttctgcctttatacttttatataaagaagcgatttcaaaaaattataaaatctacagattatttttttatacgttattatgttacacgctctttgaggaaatagatcctaatcattggagtattgtattattattctcaacttttggtatagtgggaagggctaaaaaatgattaactcaattaccttttaaattataggagttgaaaatgaattatagtatcattatgtcggtatataatgagcctttaaattatgtgagagattcagtagaatctatattaaatcaaacgcttactgattttgagttcataattgtcattgataatccaagtagaggtgatttaaagcaattcttaacagaatattcagttgtagataatagaataaaaatcttgcttaatgaagaaaatattggtttagcatcaagtttgaacaaagcggtgaaaatttctaagggagaatatatttttagaatggatgctgatgatatttcatatccaagtagatttgataagcaaattcgttttatggaggaaaattcattggatttctcagcaactctaatagaattgatagaccaaaaaggaaatttagtatataaacaacgagaaagtaataaaatatacttaactaatgatatacggaagatgttattgaatagatctatacttgcccacccaacgtggtgcgtaaaaaagaaagttttcgataagttaatgggatatagagatttagtacctgttgaagattatgattttgcaataagaggagctctggctgatttcaaaatcggcttactcaataaagtacttttacagtatagattaaacgagaatggaatatcacaaaccaataagtttaagcaatatatttactcagctattttacaagatttttataaagaaaaatcttatattgatatcacaaaaattactaattactttcaagagtatgtgataaagaaacgctatactcagcaagagctctctaaatattttgagctaaaatctacccctagtattactattagaaaactatatatttgtttatatttatactttaagtctcccttggttaggaggttattaataaatgatattaatattttagtactgaaattgtttggaggagagaaacaaagtgactaatagaaaaatttatgtatgtcatactctttatcatttattgatttgtttatataaagaagagatatattcaaatttagaaattattctctcttcttctattcctgatgttgataatttagagaaaaaattaaaatcaaaaacaataaatatacatattttagaagaatctagtggtgaaagtgaagaattattatcagtacttaaagatgctggtctaagttatagtaagtttgatagtaattgttttatttttaatgatgcaacgcctattgggaggacactaataaagcatggtatttattataatctaattgaagatggtttaaattgttttacttactctatatttagtcaaaaactttggaagtattatgtaaaaaaatatattcttcacaaaattcagccacatggattttcacgatattgtttagggattgaagttaattcattagttaatttgccaaaggatccgcgttataaaaaatttattgaagtccctaggaaagaactttttgacaatgtaacagaatatcaaaaagaaatggcaataaatctttttggagcagtaagagttagtattaaatcaccttcagtactagtattaacgcagcctctatctatagataaagagtttatgagttataacaataagatagaaacgtccgaagaacaatttaatttttataaatcaatagtcaatgaatatataaataaagggtacaatgtttatttaaaagttcatcctagagatgtagtagattattccaaattgccggtagagctattaccatcaaatgttcctatggaaattatagagttgatgttaacaggtcggttcgaatgtgggataacacattcgtccactgcgctggattttttaacttgtgttgataaaaaaataactttagtagatctgaaggatattaaatgagcggccgcatcgatcgatgagatctataaaaggctacttcttaagtaaccatgtttaagaaaagagaaatagccttatttctctttttgtcgtttttaaaataaaggaacatcatgaaacaaacattaaaacttatgttttcttttctgttgatgttagggactatgtttggaattagccaaactgttttagcgcaagaaactcatcagttgacgattgttcatcttgaagcaagggatattgatcgtccaaatccacagttggagattgcccctaaagaagggactccaattgaaggagtactctatcagttgtaccaattaaaatcaactgaagatggcgatgtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttc

SEQ ID NO: 49 - DNA, 3102 bp, Streptococcus agalactiae
gcggccgcattataggtgaaatatgaggaaatatctagatttagattattctttattttatgctctttgggtacttattttagtaccaaaccaatggtatcagtttttaattattaccattatagttctattattactttggaagagtgagtttagaatatctataagcaattcttcaatactatttctgctttggttatttatttatttatttgcaatactcattagaggtactcaagaggatataacgtttcagcgatttattgctgagctattaaaactaattagtacaggatatgctttatttttttataattattatagaaaagctgattttaatagttcagttgtaaggaatgtggtaaaggttaactattttgtgttgtttcttataacagttttatatttattttttcctatgctgaagccaactttatttggaagagaattgttttcaatagagtggtttccacatatgagaataagacttgcggcatattttgaatatgctacactaattggtcagtttattttattttcttatcccatactttttttgaaaccccaaaaacatatggaaaatattttaatatccttactgttgactatatgttcatacttttctggcgctagaatactattggtctgtatgttggttttattagcatcgcttcttttagattatatcctttttaaaactaatttgaaattgaccaagaaaaacacttttatacttggtatgactttcttatttatcaccgcttgtttttcttataacatatggtcaataattgaaaaaataattatgtacagaaaccaaagtactatcactaggatgatagtttatcaagaaagtattattgaagttctaaaaggaaatattttatttggacagggtataaggattccatcaagtgaaggaatattcctaggatcgcattctacttatattagtgtcttttacaggacttctttattaggaattgttctttatttttctgcctttatacttttatataaagaagcgatttcaaaaaattataaaatctacagattatttttttatacgttattatgttacacgctctttgaggaaatagatcctaatcattggagtattgtattattattctcaacttttggtatagtgggaagggctaaaaaatgattaactcaattaccttttaaattataggagttgaaaatgaattatagtatcattatgtcggtatataatgagcctttaaattatgtgagagattcagtagaatctatattaaatcaaacgcttactgattttgagttcataattgtcattgataatccaagtagaggtgatttaaagcaattcttaacagaatattcagttgtagataatagaataaaaatcttgcttaatgaagaaaatattggtttagcatcaagtttgaacaaagcggtgaaaatttctaagggagaatatatttttagaatggatgctgatgatatttcatatccaagtagatttgataagcaaattcgttttatggaggaaaattcattggatttctcagcaactctaatagaattgatagaccaaaaaggaaatttagtatataaacaacgagaaagtaataaaatatacttaactaatgatatacggaagatgttattgaatagatctatacttgcccacccaacgtggtgcgtaaaaaagaaagttttcgataagttaatgggatatagagatttagtacctgttgaagattatgattttgcaataagaggagctctggctgatttcaaaatcggcttactcaataaagtacttttacagtatagattaaacgagaatggaatatcacaaaccaataagtttaagcaatatatttactcagctattttacaagatttttataaagaaaaatcttatattgatatcacaaaaattactaattactttcaagagtatgtgataaagaaacgctatactcagcaagagctctctaaatattttgagctaaaatctacccctagtattactattagaaaactatatatttgtttatatttatactttaagtctcccttggttaggaggttattaataaatgatattaatattttagtactgaaattgtttggaggagagaaacaaagtgactaatagaaaaatttatgtatgtcatactctttatcatttattgatttgtttatataaagaagagatatattcaaatttagaaattattctctcttcttctattcctgatgttgataatttagagaaaaaattaaaatcaaaaacaataaatatacatattttagaagaatctagtggtgaaagtgaagaattattatcagtacttaaagatgctggtctaagttatagtaagtttgatagtaattgttttatttttaatgatgcaacgcctattgggaggacactaataaagcatggtatttattataatctaattgaagatggtttaaattgttttacttactctatatttagtcaaaaactttggaagtattatgtaaaaaaatatattcttcacaaaattcagccacatggattttcacgatattgtttagggattgaagttaattcattagttaatttgccaaaggatccgcgttataaaaaatttattgaagtccctaggaaagaactttttgacaatgtaacagaatatcaaaaagaaatggcaataaatctttttggagcagtaagagttagtattaaatcaccttcagtactagtattaacgcagcctctatctatagataaagagtttatgagttataacaataagatagaaacgtccgaagaacaatttaatttttataaatcaatagtcaatgaatatataaataaagggtacaatgtttatttaaaagttcatcctagagatgtagtagattattccaaattgccggtagagctattaccatcaaatgttcctatggaaattatagagttgatgttaacaggtcggttcgaatgtgggataacacattcgtccactgcgctggattttttaacttgtgttgataaaaaaataactttagtagatctgaaggatattaaatgagcggccgcatcgatcgatgagatct

SEQ ID NO: 50 - DNA, 13611 bp, Streptococcus agalactiae
ttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgactttaaacgtggatcattttctttaaatttatgctgacgacctttgaatttgccttttttcttagcaatttcgattccttgtgcctgacgttccttaattttttttcgttctgattctgcttgatacttgtacaattcaatgacaaggctattaatcaaacgccttaaattttcatcttcaataccattcattgagggtaaatttaagacttccagggttgcccccttaatttgaatttgattcatcaattctgttaattctttattatttcgtcctaatcgatctaattcagtaacaataacaatatccccttcacgaatatagttaagcatagcttgtaattgtgggcgttcgaccgattgaccgcttaatttgtctgaaaagaccttagaaacgccctgtaacgcttgtaattgccgatctaagttctgttctttgctactgacacgtgcataaccaattttagccattttcaaccaacctctaaaattctctcggttgcaataaccaatcagcaatatctactttttcaatttcaaattgcttatcagaaattgtcttttcgtaagcgataaaatcttgcgcatattgttgctcattaaaaatagccaccacttcgtcattttctaaaactcgataaataaattttttcattttactcctcctattatgcccaacttaaatgacctattcaccaagtcaattatactgctaaaatcatattaggacaaataggtatactctattgacctataaatgatagcaacttaaaagatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcactaaacagcaaagaaaaacaaacacgtatcatagatataaatgtaatggcatagtgcgggttttattttcagcctgtatcgtagctaaacaaatcgagttgtgggtccgttttggggcgttctgccaatttgtttagagtttcttgaataaatgtacgttctaaattaaacgaagctgtcagcgcctttatatagctttctcgttcttctttttttaatttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcgctaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacataacgctattgcgccctactaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattgggatcaaaaaattctacattgtccgttcttggtatacgagcaatcccaaaatgattgcacgttagatcaactggcaaagactttccaaaatattctcggatattttgcgagattattttggctgctttgacagatttaaattctgattttgaagtcacatagactggcgtttctaaaacaaaatatgcttgataacctttatcagatttgataattaacgtaggcataaaacctaaatcaatagctgttgttaaaatatcgcttgctgaaatagtttctttttccgtgtgaatatcaaaatcaataaagaaggtattgatttgtcttaaattgttttcagaatgtcctttagtgtatgaacggttttcgtctgcatacgtaccataacgataaacgtttggtgtccaatgcgtaaatgtatcttgattttcgtgaatcgcttcttcggaagtcagaacaacgccacgtccgccaatcatgcttttttttgagcgatacgcaaaaatagcccctttacttttacctggcttggtagtgattgagcgaattttactatttttaaatttgtactttaacaagccgtcatgaagcacagtttctacaacaaaagggatattcattcagctgttctcctttcttacgaaaattaattagttagaagctacgatcaaagttgaatcacaacaaaaaaggcaatcaactaagtttttcttaattgattgcctggtatcttcttaaagacttgaaatcccctcaaaaacccgatataatgggtttacagatatttaagtatctgattaataaagtaattaaatactttaccaaattttgggtctcgacttctttaattgattggtggtaatcaattaaggctcgcagttaaaatttctcaggctttaactggtcgtggctctttttttgtattctttattcagttcgttgtttcgttatatctagtatatcgctttttaaaaaaataagcaatgatttcgtgcattattcacacgaaatcattgcttttttcttcttccatttctaactccaatgttacttgttctgtttctggttctggttctgttggctcatttgggattaaatccactactagcgttgagttagttccgtctctaatagccggttaagtaatagccggttaagtggtcaaactttgggaaaatctcaacccgcattaagttttgatgccatgacaatcgttggaaatttgaacaaaactaatgctaaaaagctatctgactttatgagtgtagagccacaaatacgactttgggatatacttcaaacaaagtttaaagctaaggcacttcaagaaaaagtttatatcgaatatgacaaagtaaaagcagatacttgggatagacgtaatatgcgtgttgaatttaatcccaataaactcacacatgaagaaatgatttggttaaaacaaaatattatcgactacatggaagatgacggttttacaagattagacttagcttttgattttgaagatgatttgagcgattactatgcaatgactgataaagcagttaagaaaactgttttttatggtcgtaatggcaagccagaaacaaaatattttggtgtccgtgatagtgatagatttattagaatttataataaaaaacaagaacgtaaagataacgcagatgttgaagttgtgtttgaacatttatggcgtgtagaagttgaattaaaaagagatatggttgattactggaatgattgttttaatgatttacacatctttgaaacctgcgtgggctactttagaaaaaattaatgagcaagctatggtttatactttgttgcatgaagaaagtatgtggggaaagctaagtaagaatactaagactaaatttaaaaaattgattagagaaatatctccaattgatttaacggaattaatgaaatcgactttaaaagcgaacgaaaaacaattgcaaaagcagattgatttttggcaacgtgaatttaggttttggaagtaaaataagttttatttgataaaaattgctaattcagtataattaatatttacgaggtgacataacgtatgaaaaaatcagaggattattcctcctaaatataaaaatttaaaatttaggaggaagttatatatgacttttaatattattgaattagaaaattgggatagaaaagaatattttgaacactattttaatcagcaaactacttatagcattactaaagaaattgatattactttgtttaaagatatgataaaaaagaaaggatatgaaatttatccctctttaatttatgcaattatggaagttgtaaataaaaataaagtgtttagaacaggaattaatagtgagaataaattaggttattgggataagttaaatcctttgtatacagtttttaataagcaaactgaaaaatttactaacatttggactgaatctgataaaaacttcatttctttttataataattataaaaatgacttgcttgaatataaagataaagaagaaatgtttcctaaaaaaccgatacctgaaaacaccataccgatttcaatgattccttggattgattttagttcatttaatttaaatattggtaacaatagcagctttttattgcctattattacgataggtaaattttatagtgagaataataaaatttatataccagttgctctgcaacttcatcattctgtatgtgatggttaccatgcttcactatttatgaatgaatttcaagatataattcatagggtagatgattggatttagtttttagattttgaaagtgaatttaattttatacacgtaagtgatcataaaatttatgaacgtataacaaccacattttttggttgcttgtggttttgattttgaatttggttttgaacttatggactgatttattcagtccattttttgtgcttgcacaaaaactagcctcgcagagcacacgcattaatgacttatgaaacgtagtaaataagtctagtgtgttatactttacttggaagatgcaccgaataaaaaatattgaagaacaactagcaaaagattttaaagagttattttattttaagtctttataacatgagtgaagcgaatttttaaatttcgatagaaatttttacatcaaaaagccccctgtcaaaattgacgaagggggttttttggcgcacgcttttcgttagaaatatacaagattgaaaatcgtgtataagtgcgccctttgttttgaacttagcacgttacatcaattttttaaaatgatgtataagtgcgcccttttaaattttgagtgattatattttttgagttagaaaaagggattgggaaaatttcccaaaataatttaaaaaataagcaaaaattttcgatagagaatgtgctattttttgtcaaaggtgtataccttgactgtgcttgctgttacattaagtttatttttaagttattaaaaaagaaatagcttttaaagtttggctcgctgtcgctttataaagctgattgacttttgattgcaaactacttaaagaaaacaaactcggactattcgttttcttctctttggtttgaacatcagcaattatcccctcttgattgcctattttagcttgtttagaagaaacaaaagctaaaagctcctcttgggttttaaaacgctgtgtggggcttagaacgcccttaaacgacccttggtttacttttatactagcttccacctcgaaaaaaggttcttttttaaaattctctatggcttcctggcgctgaaaaaataaggtataaggtgggcgtttgaacacgtcctagtgaaaatgtaccttgtacgccccttctgttgtaaatttaacgtatacaaagggcttgcgttcatgccgatcaaccaatcggcaatttggcgtgtttgcgcttcttgataaaagggatagtaattcattccaggttgcaaattttgaaaaccgcttcggattacatctttttctaagctattgatccatagtcttttaaatgttttatcttttgaaaaggcatttgctttatggataatcgaccaggcgatattttcaccttctctgtcgctatctgttgcaacaataattgtatttgcctttttgagaagttctgcaacaattttaaactgctttcccttatcttttgcaacttcaaaatcgtatcgatcaggaaaaatcggcaaagattcaagtttccaattttgccacttttcgtcataatgacctggttctgctaattccactaaatgcccaaaaccaaaggtgataaacgtttcatctgtaaatagtgggtctttgatctcaaaataaccgtcttttttggtgctttgttttaaagcacttgcgtaggctaatgcctggcttggtttttcagctaaaataaccgtactcattaactatccctcttttcattgttttttctttgatcgactgtcacgttatatcttgctcgataccttctaaacgttcggcgattgattccagtttgttcttcaacttctttatcggataaaccattcaaaaacaaatcgaaagccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgtgacatgactgaaataggtagttgagatatggtatgcaatgtttgaacatgtttaaaatcgaatgtaaccatttgatagaccgccttcattatcatttctagaatttttctttaggtttgtaaagactacaaaataaaatgatgaaaacaactatcttgtggatacactaaaaagacacgctaattagcaaactctctcttcatcatctctcaccattattatactactatttatatgacaaataaaggttattttgttaaaatataactttgaaaatccacatatatttttaatcttccgtctgaaaaaataaataaaaatagtaaaaataaacacgaatttaaaataagcaaattttttaagaaaatctgtgctaaactttaatagttttgtgcttaataataatcagcacttacaaagaacaaagggaagcggccgcattataggtgaaatatgaggaaatatctagatttagattattctttattttatgctctttgggtacttattttagtaccaaaccaatggtatcagtttttaattattaccattatagttctattattactttggaagagtgagtttagaatatctataagcaattcttcaatactatttctgctttggttatttatttatttatttgcaatactcattagaggtactcaagaggatataacgtttcagcgatttattgctgagctattaaaactaattagtacaggatatgctttatttttttataattattatagaaaagctgattttaatagttcagttgtaaggaatgtggtaaaggttaactattttgtgttgtttcttataacagttttatatttattttttcctatgctgaagccaactttatttggaagagaattgttttcaatagagtggtttccacatatgagaataagacttgcggcatattttgaatatgctacactaattggtcagtttattttattttcttatcccatactttttttgaaaccccaaaaacatatggaaaatattttaatatccttactgttgactatatgttcatacttttctggcgctagaatactattggtctgtatgttggttttattagcatcgcttcttttagattatatcctttttaaaactaatttgaaattgaccaagaaaaacacttttatacttggtatgactttcttatttatcaccgcttgtttttcttataacatatggtcaataattgaaaaaataattatgtacagaaaccaaagtactatcactaggatgatagtttatcaagaaagtattattgaagttctaaaaggaaatattttatttggacagggtataaggattccatcaagtgaaggaatattcctaggatcgcattctacttatattagtgtcttttacaggacttctttattaggaattgttctttatttttctgcctttatacttttatataaagaagcgatttcaaaaaattataaaatctacagattatttttttatacgttattatgttacacgctctttgaggaaatagatcctaatcattggagtattgtattattattctcaacttttggtatagtgggaagggctaaaaaatgattaactcaattaccttttaaattataggagttgaaaatgaattatagtatcattatgtcggtatataatgagcctttaaattatgtgagagattcagtagaatctatattaaatcaaacgcttactgattttgagttcataattgtcattgataatccaagtagaggtgatttaaagcaattcttaacagaatattcagttgtagataatagaataaaaatcttgcttaatgaagaaaatattggtttagcatcaagtttgaacaaagcggtgaaaatttctaagggagaatatatttttagaatggatgctgatgatatttcatatccaagtagatttgataagcaaattcgttttatggaggaaaattcattggatttctcagcaactctaatagaattgatagaccaaaaaggaaatttagtatataaacaacgagaaagtaataaaatatacttaactaatgatatacggaagatgttattgaatagatctatacttgcccacccaacgtggtgcgtaaaaaagaaagttttcgataagttaatgggatatagagatttagtacctgttgaagattatgattttgcaataagaggagctctggctgatttcaaaatcggcttactcaataaagtacttttacagtatagattaaacgagaatggaatatcacaaaccaataagtttaagcaatatatttactcagctattttacaagatttttataaagaaaaatcttatattgatatcacaaaaattactaattactttcaagagtatgtgataaagaaacgctatactcagcaagagctctctaaatattttgagctaaaatctacccctagtattactattagaaaactatatatttgtttatatttatactttaagtctcccttggttaggaggttattaataaatgatattaatattttagtactgaaattgtttggaggagagaaacaaagtgactaagcggccgcatcgatcgatgagatctataaaaggctacttcttaagtaaccatgtttaagaaaagagaaatagccttatttctctttttgtcgtttttaaaataaaggaacatcatgaaacaaacattaaaacttatgttttcttttctgttgatgttagggactatgtttggaattagccaaactgttttagcgcaagaaactcatcagttgacgattgttcatcttgaagcaagggatattgatcgtccaaatccacagttggagattgcccctaaagaagggactccaattgaaggagtactctatcagttgtaccaattaaaatcaactgaagatggcgatgtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttc

SEQ ID NO: 51 - DNA, 2171 bp, Streptococcus agalactiae
gcggccgcattataggtgaaatatgaggaaatatctagatttagattattctttattttatgctctttgggtacttattttagtaccaaaccaatggtatcagtttttaattattaccattatagttctattattactttggaagagtgagtttagaatatctataagcaattcttcaatactatttctgctttggttatttatttatttatttgcaatactcattagaggtactcaagaggatataacgtttcagcgatttattgctgagctattaaaactaattagtacaggatatgctttatttttttataattattatagaaaagctgattttaatagttcagttgtaaggaatgtggtaaaggttaactattttgtgttgtttcttataacagttttatatttattttttcctatgctgaagccaactttatttggaagagaattgttttcaatagagtggtttccacatatgagaataagacttgcggcatattttgaatatgctacactaattggtcagtttattttattttcttatcccatactttttttgaaaccccaaaaacatatggaaaatattttaatatccttactgttgactatatgttcatacttttctggcgctagaatactattggtctgtatgttggttttattagcatcgcttcttttagattatatcctttttaaaactaatttgaaattgaccaagaaaaacacttttatacttggtatgactttcttatttatcaccgcttgtttttcttataacatatggtcaataattgaaaaaataattatgtacagaaaccaaagtactatcactaggatgatagtttatcaagaaagtattattgaagttctaaaaggaaatattttatttggacagggtataaggattccatcaagtgaaggaatattcctaggatcgcattctacttatattagtgtcttttacaggacttctttattaggaattgttctttatttttctgcctttatacttttatataaagaagcgatttcaaaaaattataaaatctacagattatttttttatacgttattatgttacacgctctttgaggaaatagatcctaatcattggagtattgtattattattctcaacttttggtatagtgggaagggctaaaaaatgattaactcaattaccttttaaattataggagttgaaaatgaattatagtatcattatgtcggtatataatgagcctttaaattatgtgagagattcagtagaatctatattaaatcaaacgcttactgattttgagttcataattgtcattgataatccaagtagaggtgatttaaagcaattcttaacagaatattcagttgtagataatagaataaaaatcttgcttaatgaagaaaatattggtttagcatcaagtttgaacaaagcggtgaaaatttctaagggagaatatatttttagaatggatgctgatgatatttcatatccaagtagatttgataagcaaattcgttttatggaggaaaattcattggatttctcagcaactctaatagaattgatagaccaaaaaggaaatttagtatataaacaacgagaaagtaataaaatatacttaactaatgatatacggaagatgttattgaatagatctatacttgcccacccaacgtggtgcgtaaaaaagaaagttttcgataagttaatgggatatagagatttagtacctgttgaagattatgattttgcaataagaggagctctggctgatttcaaaatcggcttactcaataaagtacttttacagtatagattaaacgagaatggaatatcacaaaccaataagtttaagcaatatatttactcagctattttacaagatttttataaagaaaaatcttatattgatatcacaaaaattactaattactttcaagagtatgtgataaagaaacgctatactcagcaagagctctctaaatattttgagctaaaatctacccctagtattactattagaaaactatatatttgtttatatttatactttaagtctcccttggttaggaggttattaataaatgatattaatattttagtactgaaattgtttggaggagagaaacaaagtgactaagcggccgcatcgatcgatgagatct

SEQ ID NO: 52 - DNA, 14129 bp, Streptococcus agalactiae
ttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgactttaaacgtggatcattttctttaaatttatgctgacgacctttgaatttgccttttttcttagcaatttcgattccttgtgcctgacgttccttaattttttttcgttctgattctgcttgatacttgtacaattcaatgacaaggctattaatcaaacgccttaaattttcatcttcaataccattcattgagggtaaatttaagacttccagggttgcccccttaatttgaatttgattcatcaattctgttaattctttattatttcgtcctaatcgatctaattcagtaacaataacaatatccccttcacgaatatagttaagcatagcttgtaattgtgggcgttcgaccgattgaccgcttaatttgtctgaaaagaccttagaaacgccctgtaacgcttgtaattgccgatctaagttctgttctttgctactgacacgtgcataaccaattttagccattttcaaccaacctctaaaattctctcggttgcaataaccaatcagcaatatctactttttcaatttcaaattgcttatcagaaattgtcttttcgtaagcgataaaatcttgcgcatattgttgctcattaaaaatagccaccacttcgtcattttctaaaactcgataaataaattttttcattttactcctcctattatgcccaacttaaatgacctattcaccaagtcaattatactgctaaaatcatattaggacaaataggtatactctattgacctataaatgatagcaacttaaaagatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcactaaacagcaaagaaaaacaaacacgtatcatagatataaatgtaatggcatagtgcgggttttattttcagcctgtatcgtagctaaacaaatcgagttgtgggtccgttttggggcgttctgccaatttgtttagagtttcttgaataaatgtacgttctaaattaaacgaagctgtcagcgcctttatatagctttctcgttcttctttttttaatttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcgctaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacataacgctattgcgccctactaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattgggatcaaaaaattctacattgtccgttcttggtatacgagcaatcccaaaatgattgcacgttagatcaactggcaaagactttccaaaatattctcggatattttgcgagattattttggctgctttgacagatttaaattctgattttgaagtcacatagactggcgtttctaaaacaaaatatgcttgataacctttatcagatttgataattaacgtaggcataaaacctaaatcaatagctgttgttaaaatatcgcttgctgaaatagtttctttttccgtgtgaatatcaaaatcaataaagaaggtattgatttgtcttaaattgttttcagaatgtcctttagtgtatgaacggttttcgtctgcatacgtaccataacgataaacgtttggtgtccaatgcgtaaatgtatcttgattttcgtgaatcgcttcttcggaagtcagaacaacgccacgtccgccaatcatgcttttttttgagcgatacgcaaaaatagcccctttacttttacctggcttggtagtgattgagcgaattttactatttttaaatttgtactttaacaagccgtcatgaagcacagtttctacaacaaaagggatattcattcagctgttctcctttcttacgaaaattaattagttagaagctacgatcaaagttgaatcacaacaaaaaaggcaatcaactaagtttttcttaattgattgcctggtatcttcttaaagacttgaaatcccctcaaaaacccgatataatgggtttacagatatttaagtatctgattaataaagtaattaaatactttaccaaattttgggtctcgacttctttaattgattggtggtaatcaattaaggctcgcagttaaaatttctcaggctttaactggtcgtggctctttttttgtattctttattcagttcgttgtttcgttatatctagtatatcgctttttaaaaaaataagcaatgatttcgtgcattattcacacgaaatcattgcttttttcttcttccatttctaactccaatgttacttgttctgtttctggttctggttctgttggctcatttgggattaaatccactactagcgttgagttagttccgtctctaatagccggttaagtaatagccggttaagtggtcaaactttgggaaaatctcaacccgcattaagttttgatgccatgacaatcgttggaaatttgaacaaaactaatgctaaaaagctatctgactttatgagtgtagagccacaaatacgactttgggatatacttcaaacaaagtttaaagctaaggcacttcaagaaaaagtttatatcgaatatgacaaagtaaaagcagatacttgggatagacgtaatatgcgtgttgaatttaatcccaataaactcacacatgaagaaatgatttggttaaaacaaaatattatcgactacatggaagatgacggttttacaagattagacttagcttttgattttgaagatgatttgagcgattactatgcaatgactgataaagcagttaagaaaactgttttttatggtcgtaatggcaagccagaaacaaaatattttggtgtccgtgatagtgatagatttattagaatttataataaaaaacaagaacgtaaagataacgcagatgttgaagttgtgtttgaacatttatggcgtgtagaagttgaattaaaaagagatatggttgattactggaatgattgttttaatgatttacacatctttgaaacctgcgtgggctactttagaaaaaattaatgagcaagctatggtttatactttgttgcatgaagaaagtatgtggggaaagctaagtaagaatactaagactaaatttaaaaaattgattagagaaatatctccaattgatttaacggaattaatgaaatcgactttaaaagcgaacgaaaaacaattgcaaaagcagattgatttttggcaacgtgaatttaggttttggaagtaaaataagttttatttgataaaaattgctaattcagtataattaatatttacgaggtgacataacgtatgaaaaaatcagaggattattcctcctaaatataaaaatttaaaatttaggaggaagttatatatgacttttaatattattgaattagaaaattgggatagaaaagaatattttgaacactattttaatcagcaaactacttatagcattactaaagaaattgatattactttgtttaaagatatgataaaaaagaaaggatatgaaatttatccctctttaatttatgcaattatggaagttgtaaataaaaataaagtgtttagaacaggaattaatagtgagaataaattaggttattgggataagttaaatcctttgtatacagtttttaataagcaaactgaaaaatttactaacatttggactgaatctgataaaaacttcatttctttttataataattataaaaatgacttgcttgaatataaagataaagaagaaatgtttcctaaaaaaccgatacctgaaaacaccataccgatttcaatgattccttggattgattttagttcatttaatttaaatattggtaacaatagcagctttttattgcctattattacgataggtaaattttatagtgagaataataaaatttatataccagttgctctgcaacttcatcattctgtatgtgatggttaccatgcttcactatttatgaatgaatttcaagatataattcatagggtagatgattggatttagtttttagattttgaaagtgaatttaattttatacacgtaagtgatcataaaatttatgaacgtataacaaccacattttttggttgcttgtggttttgattttgaatttggttttgaacttatggactgatttattcagtccattttttgtgcttgcacaaaaactagcctcgcagagcacacgcattaatgacttatgaaacgtagtaaataagtctagtgtgttatactttacttggaagatgcaccgaataaaaaatattgaagaacaactagcaaaagattttaaagagttattttattttaagtctttataacatgagtgaagcgaatttttaaatttcgatagaaatttttacatcaaaaagccccctgtcaaaattgacgaagggggttttttggcgcacgcttttcgttagaaatatacaagattgaaaatcgtgtataagtgcgccctttgttttgaacttagcacgttacatcaattttttaaaatgatgtataagtgcgcccttttaaattttgagtgattatattttttgagttagaaaaagggattgggaaaatttcccaaaataatttaaaaaataagcaaaaattttcgatagagaatgtgctattttttgtcaaaggtgtataccttgactgtgcttgctgttacattaagtttatttttaagttattaaaaaagaaatagcttttaaagtttggctcgctgtcgctttataaagctgattgacttttgattgcaaactacttaaagaaaacaaactcggactattcgttttcttctctttggtttgaacatcagcaattatcccctcttgattgcctattttagcttgtttagaagaaacaaaagctaaaagctcctcttgggttttaaaacgctgtgtggggcttagaacgcccttaaacgacccttggtttacttttatactagcttccacctcgaaaaaaggttcttttttaaaattctctatggcttcctggcgctgaaaaaataaggtataaggtgggcgtttgaacacgtcctagtgaaaatgtaccttgtacgccccttctgttgtaaatttaacgtatacaaagggcttgcgttcatgccgatcaaccaatcggcaatttggcgtgtttgcgcttcttgataaaagggatagtaattcattccaggttgcaaattttgaaaaccgcttcggattacatctttttctaagctattgatccatagtcttttaaatgttttatcttttgaaaaggcatttgctttatggataatcgaccaggcgatattttcaccttctctgtcgctatctgttgcaacaataattgtatttgcctttttgagaagttctgcaacaattttaaactgctttcccttatcttttgcaacttcaaaatcgtatcgatcaggaaaaatcggcaaagattcaagtttccaattttgccacttttcgtcataatgacctggttctgctaattccactaaatgcccaaaaccaaaggtgataaacgtttcatctgtaaatagtgggtctttgatctcaaaataaccgtcttttttggtgctttgttttaaagcacttgcgtaggctaatgcctggcttggtttttcagctaaaataaccgtactcattaactatccctcttttcattgttttttctttgatcgactgtcacgttatatcttgctcgataccttctaaacgttcggcgattgattccagtttgttcttcaacttctttatcggataaaccattcaaaaacaaatcgaaagccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgtgacatgactgaaataggtagttgagatatggtatgcaatgtttgaacatgtttaaaatcgaatgtaaccatttgatagaccgccttcattatcatttctagaatttttctttaggtttgtaaagactacaaaataaaatgatgaaaacaactatcttgtggatacactaaaaagacacgctaattagcaaactctctcttcatcatctctcaccattattatactactatttatatgacaaataaaggttattttgttaaaatataactttgaaaatccacatatatttttaatcttccgtctgaaaaaataaataaaaatagtaaaaataaacacgaatttaaaataagcaaattttttaagaaaatctgtgctaaactttaatagttttgtgcttaataataatcagcacttacaaagaacaaagggaagcggccgcgctctgatatggcaggaggtaaggaaggaaaatgatacctaaagttatacattattgttggtttggaggaaatcccttaccagataatttaaagaaatatataaaaacttggagagaacaatgtccggattatgaaattattgaatggaatgagcataattatgatgttagtaaaaatgtttttatgagagaagcatatactaagaagaattttgcttatgtttctgactatgcaagattggatattatttatacttatggggggttctatctagatactgatgtggagcttttaaaaagtttagatcctttgaggattcatgagtgttttctagcaagggagattagttgtgatgtgaatacaggattaataattggcgctgttaaaggacatcactttttaaaatcaaatatgtctatatatgacaaaagtgatttaacttctcttaataagacatgtgtagaggttacaactaatttattgataaacagagggcttaagaataagaatattattcaaaagattgatgatataacaatatatccgagaaattattttaatccaaagaatttattaacaggtaaggttgattgtctgactagtgttacctattctatacatcattacgaaggaagttggaaaagttcttcatttatttcagattctctaaagattagagtaaggctcataattgattttttatttggatatggtacttatagaatgcttctaaggtttctaaagttaaagaaatagttattattgtaatagaaaggacgaaatgttatatatttttactcctacatttaacagaggctaccgattatcctatctgtatgattctctatgtaatcagacaaataaaaattttatatggttgattgttgatgatggttctgaggacagtactaaggaaattgtaagtaattatataaaagagaataaagttagtattgtatatctgtataaacgtaatggcggtaagcattcagcctataatctagcaatgagatatatgcaaccaagtgattatcatgtatgtgtagatagtgatgattggttattagaagatgcagttgaaattatttttaaagatttagagagcctaacgttgtctaatagatatgtaggccttgtttatcctcgatattcattaaaccaaggtaataattggttgaatcccaaaatattagaagttaatattcctgatttaaaatataaatatcacttaaaaattgaaacttgtattgttattaataatgcttatttagtagatttcgaatttccttgtttcgaaggggagaatttcttatcagaagaaataatgtatatatatttatcaaaaaagggatacttttgtccccaaaataggaaaatctattgttttgactacttagaagatggtttgacttctaatatttttaaattatggagaaaaaatttcaaagggactattttttcattagaaaattcatatatgtatgtaatgtcttttcctaatatatttgatcgctggtggtcagctataaagataaaaatgaacatacaggctttgaaaatgactactttaggggtaatacctacactaaagagtgaagaggctgggtggaaaattttgttgggttttagctatctttggaaggtagcaaggtttaaaaagagcgagtaaagaatggaattaatttcaataatagtgcctgtatataatggagaaatatatattgggagatgtctagatagtattcttgagcaaacttatcagaatttagagattatcataattgatgatggctctagtgatcgcacaggggatatttgcgaaaagtatttattggaagacaggcgaataaaatatttctatcaagaaaacagaggccaatcagttgcacgtaacaacggagtacttaggtgtacaggagattggattgcttttttagacagtgatgatgtttatcttccttattcaattgaggttatgtacaacatacagaaagctaccaatgccgatattgttttaactagtattggtaacttcaacaatacttacaatactagtattaattcacagtatttaaaagagattaaactttatactttagaggtggcgttggaagaaatgtattatgggaaaacttatggagtttccccgttagcaaaattatatccgagaagtaatttattgagtaatccatatccagaaggaaaaattcatgaagatatggatacaacttttaagcttatatcttgcgcttcaaaaatagcagtttgtgatattgtcactgcagtggtgtattttagcgataatagtactactaggacaaaattcaatgagagaatgttgtatttttttgaggcaattcaaaataatattgtatttataaatctaaattttccacataatacatctttaatctctgctgtaatatataatgaagtatttggtggaatagatatatgtgggaagatgattgattttaaattatatgatacagttgattattacagaaaaaaatatagaaaatattttaagactattctctttaataataggattagtgtgaaggagaaggttaaatatatattatttatttcctctataagatatttcactattgtaagaaaaatatataatttgaggttatcaaggaatggataaagttagtatcattatcccagatctataaaaggctacttcttaagtaaccatgtttaagaaaagagaaatagccttatttctctttttgtcgtttttaaaataaaggaacatcatgaaacaaacattaaaacttatgttttcttttctgttgatgttagggactatgtttggaattagccaaactgttttagcgcaagaaactcatcagttgacgattgttcatcttgaagcaagggatattgatcgtccaaatccacagttggagattgcccctaaagaagggactccaattgaaggagtactctatcagttgtaccaattaaaatcaactgaagatggcgatgtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttc

SEQ ID NO: 53 - DNA, 2689 bp, Streptococcus agalactiae
gcggccgcgctctgatatggcaggaggtaaggaaggaaaatgatacctaaagttatacattattgttggtttggaggaaatcccttaccagataatttaaagaaatatataaaaacttggagagaacaatgtccggattatgaaattattgaatggaatgagcataattatgatgttagtaaaaatgtttttatgagagaagcatatactaagaagaattttgcttatgtttctgactatgcaagattggatattatttatacttatggggggttctatctagatactgatgtggagcttttaaaaagtttagatcctttgaggattcatgagtgttttctagcaagggagattagttgtgatgtgaatacaggattaataattggcgctgttaaaggacatcactttttaaaatcaaatatgtctatatatgacaaaagtgatttaacttctcttaataagacatgtgtagaggttacaactaatttattgataaacagagggcttaagaataagaatattattcaaaagattgatgatataacaatatatccgagaaattattttaatccaaagaatttattaacaggtaaggttgattgtctgactagtgttacctattctatacatcattacgaaggaagttggaaaagttcttcatttatttcagattctctaaagattagagtaaggctcataattgattttttatttggatatggtacttatagaatgcttctaaggtttctaaagttaaagaaatagttattattgtaatagaaaggacgaaatgttatatatttttactcctacatttaacagaggctaccgattatcctatctgtatgattctctatgtaatcagacaaataaaaattttatatggttgattgttgatgatggttctgaggacagtactaaggaaattgtaagtaattatataaaagagaataaagttagtattgtatatctgtataaacgtaatggcggtaagcattcagcctataatctagcaatgagatatatgcaaccaagtgattatcatgtatgtgtagatagtgatgattggttattagaagatgcagttgaaattatttttaaagatttagagagcctaacgttgtctaatagatatgtaggccttgtttatcctcgatattcattaaaccaaggtaataattggttgaatcccaaaatattagaagttaatattcctgatttaaaatataaatatcacttaaaaattgaaacttgtattgttattaataatgcttatttagtagatttcgaatttccttgtttcgaaggggagaatttcttatcagaagaaataatgtatatatatttatcaaaaaagggatacttttgtccccaaaataggaaaatctattgttttgactacttagaagatggtttgacttctaatatttttaaattatggagaaaaaatttcaaagggactattttttcattagaaaattcatatatgtatgtaatgtcttttcctaatatatttgatcgctggtggtcagctataaagataaaaatgaacatacaggctttgaaaatgactactttaggggtaatacctacactaaagagtgaagaggctgggtggaaaattttgttgggttttagctatctttggaaggtagcaaggtttaaaaagagcgagtaaagaatggaattaatttcaataatagtgcctgtatataatggagaaatatatattgggagatgtctagatagtattcttgagcaaacttatcagaatttagagattatcataattgatgatggctctagtgatcgcacaggggatatttgcgaaaagtatttattggaagacaggcgaataaaatatttctatcaagaaaacagaggccaatcagttgcacgtaacaacggagtacttaggtgtacaggagattggattgcttttttagacagtgatgatgtttatcttccttattcaattgaggttatgtacaacatacagaaagctaccaatgccgatattgttttaactagtattggtaacttcaacaatacttacaatactagtattaattcacagtatttaaaagagattaaactttatactttagaggtggcgttggaagaaatgtattatgggaaaacttatggagtttccccgttagcaaaattatatccgagaagtaatttattgagtaatccatatccagaaggaaaaattcatgaagatatggatacaacttttaagcttatatcttgcgcttcaaaaatagcagtttgtgatattgtcactgcagtggtgtattttagcgataatagtactactaggacaaaattcaatgagagaatgttgtatttttttgaggcaattcaaaataatattgtatttataaatctaaattttccacataatacatctttaatctctgctgtaatatataatgaagtatttggtggaatagatatatgtgggaagatgattgattttaaattatatgatacagttgattattacagaaaaaaatatagaaaatattttaagactattctctttaataataggattagtgtgaaggagaaggttaaatatatattatttatttcctctataagatatttcactattgtaagaaaaatatataatttgaggttatcaaggaatggataaagttagtatcattatcccagatct

SEQ ID NO: 54 - DNA, 41 bp, Artificial Sequence
  PCR Primer
gcggcagatctgtaaggaaggaaaatgatacctaaagttat

SEQ ID NO: 55 - DNA, 15918 bp, Artificial Sequence
  Plasmid sequence
ctttaaacgtggatcattttctttaaatttatgctgacgacctttgaatttgccttttttcttagcaatttcgattccttgtgcctgacgttccttaattttttttcgttctgattctgcttgatacttgtacaattcaatgacaaggctattaatcaaacgccttaaattttcatcttcaataccattcattgagggtaaatttaagacttccagggttgcccccttaatttgaatttgattcatcaattctgttaattctttattatttcgtcctaatcgatctaattcagtaacaataacaatatccccttcacgaatatagttaagcatagcttgtaattgtgggcgttcgaccgattgaccgcttaatttgtctgaaaagaccttagaaacgccctgtaacgcttgtaattgccgatctaagttctgttctttgctactgacacgtgcataaccaattttagccattttcaaccaacctctaaaattctctcggttgcaataaccaatcagcaatatctactttttcaatttcaaattgcttatcagaaattgtcttttcgtaagcgataaaatcttgcgcatattgttgctcattaaaaatagccaccacttcgtcattttctaaaactcgataaataaattttttcattttactcctcctattatgcccaacttaaatgacctattcaccaagtcaattatactgctaaaatcatattaggacaaataggtatactctattgacctataaatgatagcaacttaaaagatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcactaaacagcaaagaaaaacaaacacgtatcatagatataaatgtaatggcatagtgcgggttttattttcagcctgtatcgtagctaaacaaatcgagttgtgggtccgttttggggcgttctgccaatttgtttagagtttcttgaataaatgtacgttctaaattaaacgaagctgtcagcgcctttatatagctttctcgttcttctttttttaatttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcgctaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacataacgctattgcgccctactaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattgggatcaaaaaattctacattgtccgttcttggtatacgagcaatcccaaaatgattgcacgttagatcaactggcaaagactttccaaaatattctcggatattttgcgagattattttggctgctttgacagatttaaattctgattttgaagtcacatagactggcgtttctaaaacaaaatatgcttgataacctttatcagatttgataattaacgtaggcataaaacctaaatcaatagctgttgttaaaatatcgcttgctgaaatagtttctttttccgtgtgaatatcaaaatcaataaagaaggtattgatttgtcttaaattgttttcagaatgtcctttagtgtatgaacggttttcgtctgcatacgtaccataacgataaacgtttggtgtccaatgcgtaaatgtatcttgattttcgtgaatcgcttcttcggaagtcagaacaacgccacgtccgccaatcatgcttttttttgagcgatacgcaaaaatagcccctttacttttacctggcttggtagtgattgagcgaattttactatttttaaatttgtactttaacaagccgtcatgaagcacagtttctacaacaaaagggatattcattcagctgttctcctttcttacgaaaattaattagttagaagctacgatcaaagttgaatcacaacaaaaaaggcaatcaactaagtttttcttaattgattgcctggtatcttcttaaagacttgaaatcccctcaaaaacccgatataatgggtttacagatatttaagtatctgattaataaagtaattaaatactttaccaaattttgggtctcgacttctttaattgattggtggtaatcaattaaggctcgcagttaaaatttctcaggctttaactggtcgtggctctttttttgtattctttattcagttcgttgtttcgttatatctagtatatcgctttttaaaaaaataagcaatgatttcgtgcattattcacacgaaatcattgcttttttcttcttccatttctaactccaatgttacttgttctgtttctggttctggttctgttggctcatttgggattaaatccactactagcgttgagttagttccgtctctaatagccggttaagtaatagccggttaagtggtcaaactttgggaaaatctcaacccgcattaagttttgatgccatgacaatcgttggaaatttgaacaaaactaatgctaaaaagctatctgactttatgagtgtagagccacaaatacgactttgggatatacttcaaacaaagtttaaagctaaggcacttcaagaaaaagtttatatcgaatatgacaaagtaaaagcagatacttgggatagacgtaatatgcgtgttgaatttaatcccaataaactcacacatgaagaaatgatttggttaaaacaaaatattatcgactacatggaagatgacggttttacaagattagacttagcttttgattttgaagatgatttgagcgattactatgcaatgactgataaagcagttaagaaaactgttttttatggtcgtaatggcaagccagaaacaaaatattttggtgtccgtgatagtgatagatttattagaatttataataaaaaacaagaacgtaaagataacgcagatgttgaagttgtgtttgaacatttatggcgtgtagaagttgaattaaaaagagatatggttgattactggaatgattgttttaatgatttacacatctttgaaacctgcgtgggctactttagaaaaaattaatgagcaagctatggtttatactttgttgcatgaagaaagtatgtggggaaagctaagtaagaatactaagactaaatttaaaaaattgattagagaaatatctccaattgatttaacggaattaatgaaatcgactttaaaagcgaacgaaaaacaattgcaaaagcagattgatttttggcaacgtgaatttaggttttggaagtaaaataagttttatttgataaaaattgctaattcagtataattaatatttacgaggtgacataacgtatgaaaaaatcagaggattattcctcctaaatataaaaatttaaaatttaggaggaagttatatatgacttttaatattattgaattagaaaattgggatagaaaagaatattttgaacactattttaatcagcaaactacttatagcattactaaagaaattgatattactttgtttaaagatatgataaaaaagaaaggatatgaaatttatccctctttaatttatgcaattatggaagttgtaaataaaaataaagtgtttagaacaggaattaatagtgagaataaattaggttattgggataagttaaatcctttgtatacagtttttaataagcaaactgaaaaatttactaacatttggactgaatctgataaaaacttcatttctttttataataattataaaaatgacttgcttgaatataaagataaagaagaaatgtttcctaaaaaaccgatacctgaaaacaccataccgatttcaatgattccttggattgattttagttcatttaatttaaatattggtaacaatagcagctttttattgcctattattacgataggtaaattttatagtgagaataataaaatttatataccagttgctctgcaacttcatcattctgtatgtgatggttaccatgcttcactatttatgaatgaatttcaagatataattcatagggtagatgattggatttagtttttagattttgaaagtgaatttaattttatacacgtaagtgatcataaaatttatgaacgtataacaaccacattttttggttgcttgtggttttgattttgaatttggttttgaacttatggactgatttattcagtccattttttgtgcttgcacaaaaactagcctcgcagagcacacgcattaatgacttatgaaacgtagtaaataagtctagtgtgttatactttacttggaagatgcaccgaataaaaaatattgaagaacaactagcaaaagattttaaagagttattttattttaagtctttataacatgagtgaagcgaatttttaaatttcgatagaaatttttacatcaaaaagccccctgtcaaaattgacgaagggggttttttggcgcacgcttttcgttagaaatatacaagattgaaaatcgtgtataagtgcgccctttgttttgaacttagcacgttacatcaattttttaaaatgatgtataagtgcgcccttttaaattttgagtgattatattttttgagttagaaaaagggattgggaaaatttcccaaaataatttaaaaaataagcaaaaattttcgatagagaatgtgctattttttgtcaaaggtgtataccttgactgtgcttgctgttacattaagtttatttttaagttattaaaaaagaaatagcttttaaagtttggctcgctgtcgctttataaagctgattgacttttgattgcaaactacttaaagaaaacaaactcggactattcgttttcttctctttggtttgaacatcagcaattatcccctcttgattgcctattttagcttgtttagaagaaacaaaagctaaaagctcctcttgggttttaaaacgctgtgtggggcttagaacgcccttaaacgacccttggtttacttttatactagcttccacctcgaaaaaaggttcttttttaaaattctctatggcttcctggcgctgaaaaaataaggtataaggtgggcgtttgaacacgtcctagtgaaaatgtaccttgtacgccccttctgttgtaaatttaacgtatacaaagggcttgcgttcatgccgatcaaccaatcggcaatttggcgtgtttgcgcttcttgataaaagggatagtaattcattccaggttgcaaattttgaaaaccgcttcggattacatctttttctaagctattgatccatagtcttttaaatgttttatcttttgaaaaggcatttgctttatggataatcgaccaggcgatattttcaccttctctgtcgctatctgttgcaacaataattgtatttgcctttttgagaagttctgcaacaattttaaactgctttcccttatcttttgcaacttcaaaatcgtatcgatcaggaaaaatcggcaaagattcaagtttccaattttgccacttttcgtcataatgacctggttctgctaattccactaaatgcccaaaaccaaaggtgataaacgtttcatctgtaaatagtgggtctttgatctcaaaataaccgtcttttttggtgctttgttttaaagcacttgcgtaggctaatgcctggcttggtttttcagctaaaataaccgtactcattaactatccctcttttcattgttttttctttgatcgactgtcacgttatatcttgctcgataccttctaaacgttcggcgattgattccagtttgttcttcaacttctttatcggataaaccattcaaaaacaaatcgaaagccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgtgacatgactgaaataggtagttgagatatggtatgcaatgtttgaacatgtttaaaatcgaatgtaaccatttgatagaccgccttcattatcatttctagaatttttctttaggtttgtaaagactacaaaataaaatgatgaaaacaactatcttgtggatacactaaaaagacacgctaattagcaaactctctcttcatcatctctcaccattattatactactatttatatgacaaataaaggttattttgttaaaatataactttgaaaatccacatatatttttaatcttccgtctgaaaaaataaataaaaatagtaaaaataaacacgaatttaaaataagcaaattttttaagaaaatctgtgctaaactttaatagttttgtgcttaataataatcagcacttacaaagaacaaagggaagcggccgcgacatatttgctctgatatggcaggaggtaaggaaggaaaatgatacctaaagttatacattattgttggtttggaggaaatcccttaccagataatttaaagaaatatataaaaacttggagagaacaatgtccggattatgaaattattgaatggaatgagcataattatgatgttagtaaaaatgtttttatgagagaagcatatactaagaagaattttgcttatgtttctgactatgcaagattggatattatttatacttatggggggttctatctagatactgatgtggagcttttaaaaagtttagatcctttgaggattcatgagtgttttctagcaagggagattagttgtgatgttgccactggattaattattggtgcagttagaggacaaaaatttataaaagataatatgatggtatatgaaaaatataaatttttaaattttgataaaacttgtgtggaaattacgactgacttacttttaaaaaaaggattgaagaataaagatgtcatacaagttatagatggtgttactatttatccaagacaatattttaatcctaaaagtatgttgacggggaggttagattttctaacagatgatacatactctatacaccattatgaaggaagctggaaaaaaagatcatttgttcctgattccctaaagattagattacgtcttataatcgattttgtgttcggatatggaacatatagaaggatattgcgttttttaaaattaaaatagtagcagttattaatttaaaatattctctttaaaaaatggaggcacaatgaaagtatcattagttataccagtttataatggagcaaatcatatcaaagaaacattagaaagtattatagcgcagacctaccccaatttggaaatattaatcattgatgatgggtcaactgataatacatcaactatatgtcagaagtatgtttctaaagataagcgcatgaaatatatatataaaaaaaatggaggtcaatcttctgctagaaatttaggcgttaaactatctacaagtaattggatagctttcttagattctgatgatctttgttcgccattttatatagaatgtctagtgagaattcaacatgaaactggtgctgaccttgtaataactggaattgaggatcacccttgttacagaacggcaaaactttcatttgataaactatcttactcagtactcagtgtggaagagacactaatagaaatgtattatgataactatggtatttctccattagggaagttatattccaaagggttattattaaaatatccataccctattggtaaaattcatgaagatacagacacaacttataaactgattgcaagtagtacacgaattgcattgagcaaagagaaaacagtatatgtcaaagggcaacctaatagtactaccagacagtcatacaaagagaatatgttatattattttgaagcaatgcaacataatagagattttatccttgaacattattcagataaagaactaattgatgctttaaattataaaactgtaattggaggaatgtggatcgttaataagcttttagaaggagataagtacgattatattaattctttgagaagtgaatatagaaaatattataagagtatattatttaataagcgagttacaattaaagaaaaaataaaatatttttcatttatttcgtcaatgaatttatttttatttttaaaaaaattttataactataatgggagataaattatgaagatgattagtatcattatcccagatctgtaaggaaggaaaatgatacctaaagttatacattattgttggtttggaggaaatcccttaccagataatttaaagaaatatataaaaacttggagagaacaatgtccggattatgaaattattgaatggaatgagcataattatgatgttagtaaaaatgtttttatgagagaagcatatactaagaagaattttgcttatgtttctgactatgcaagattggatattatttatacttatggggggttctatctagatactgatgtggagcttttaaaaagtttagatcctttgaggattcatgagtgttttctagcaagggagattagttgtgatgtgaatacaggattaataattggcgctgttaaaggacatcactttttaaaatcaaatatgtctatatatgacaaaagtgatttaacttctcttaataagacatgtgtagaggttacaactaatttattgataaacagagggcttaagaataagaatattattcaaaagattgatgatataacaatatatccgagaaattattttaatccaaagaatttattaacaggtaaggttgattgtctgactagtgttacctattctatacatcattacgaaggaagttggaaaagttcttcatttatttcagattctctaaagattagagtaaggctcataattgattttttatttggatatggtacttatagaatgcttctaaggtttctaaagttaaagaaatagttattattgtaatagaaaggacgaaatgttatatatttttactcctacatttaacagaggctaccgattatcctatctgtatgattctctatgtaatcagacaaataaaaattttatatggttgattgttgatgatggttctgaggacagtactaaggaaattgtaagtaattatataaaagagaataaagttagtattgtatatctgtataaacgtaatggcggtaagcattcagcctataatctagcaatgagatatatgcaaccaagtgattatcatgtatgtgtagatagtgatgattggttattagaagatgcagttgaaattatttttaaagatttagagagcctaacgttgtctaatagatatgtaggccttgtttatcctcgatattcattaaaccaaggtaataattggttgaatcccaaaatattagaagttaatattcctgatttaaaatataaatatcacttaaaaattgaaacttgtattgttattaataatgcttatttagtagatttcgaatttccttgtttcgaaggggagaatttcttatcagaagaaataatgtatatatatttatcaaaaaagggatacttttgtccccaaaataggaaaatctattgttttgactacttagaagatggtttgacttctaatatttttaaattatggagaaaaaatttcaaagggactattttttcattagaaaattcatatatgtatgtaatgtcttttcctaatatatttgatcgctggtggtcagctataaagataaaaatgaacatacaggctttgaaaatgactactttaggggtaatacctacactaaagagtgaagaggctgggtggaaaattttgttgggttttagctatctttggaaggtagcaaggtttaaaaagagcgagtaaagaatggaattaatttcaataatagtgcctgtatataatggagaaatatatattgggagatgtctagatagtattcttgagcaaacttatcagaatttagagattatcataattgatgatggctctagtgatcgcacaggggatatttgcgaaaagtatttattggaagacaggcgaataaaatatttctatcaagaaaacagaggccaatcagttgcacgtaacaacggagtacttaggtgtacaggagattggattgcttttttagacagtgatgatgtttatcttccttattcaattgaggttatgtacaacatacagaaagctaccaatgccgatattgttttaactagtattggtaacttcaacaatacttacaatactagtattaattcacagtatttaaaagagattaaactttatactttagaggtggcgttggaagaaatgtattatgggaaaacttatggagtttccccgttagcaaaattatatccgagaagtaatttattgagtaatccatatccagaaggaaaaattcatgaagatatggatacaacttttaagcttatatcttgcgcttcaaaaatagcagtttgtgatattgtcactgcagtggtgtattttagcgataatagtactactaggacaaaattcaatgagagaatgttgtatttttttgaggcaattcaaaataatattgtatttataaatctaaattttccacataatacatctttaatctctgctgtaatatataatgaagtatttggtggaatagatatatgtgggaagatgattgattttaaattatatgatacagttgattattacagaaaaaaatatagaaaatattttaagactattctctttaataataggattagtgtgaaggagaaggttaaatatatattatttatttcctctataagatatttcactattgtaagaaaaatatataatttgaggttatcaaggaatggataaagttagtatcattatcccagatctataaaaggctacttcttaagtaaccatgtttaagaaaagagaaatagccttatttctctttttgtcgtttttaaaataaaggaacatcatgaaacaaacattaaaacttatgttttcttttctgttgatgttagggactatgtttggaattagccaaactgttttagcgcaagaaactcatcagttgacgattgttcatcttgaagcaagggatattgatcgtccaaatccacagttggagattgcccctaaagaagggactccaattgaaggagtactctatcagttgtaccaattaaaatcaactgaagatggcgatgtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccga

SEQ ID NO: 56 - DNA, 4478 bp, Artificial Sequence
  cps9M- cps9I- cps5M- cps5O- cps5I insert
gcggccgcgacatatttgctctgatatggcaggaggtaaggaaggaaaatgatacctaaagttatacattattgttggtttggaggaaatcccttaccagataatttaaagaaatatataaaaacttggagagaacaatgtccggattatgaaattattgaatggaatgagcataattatgatgttagtaaaaatgtttttatgagagaagcatatactaagaagaattttgcttatgtttctgactatgcaagattggatattatttatacttatggggggttctatctagatactgatgtggagcttttaaaaagtttagatcctttgaggattcatgagtgttttctagcaagggagattagttgtgatgttgccactggattaattattggtgcagttagaggacaaaaatttataaaagataatatgatggtatatgaaaaatataaatttttaaattttgataaaacttgtgtggaaattacgactgacttacttttaaaaaaaggattgaagaataaagatgtcatacaagttatagatggtgttactatttatccaagacaatattttaatcctaaaagtatgttgacggggaggttagattttctaacagatgatacatactctatacaccattatgaaggaagctggaaaaaaagatcatttgttcctgattccctaaagattagattacgtcttataatcgattttgtgttcggatatggaacatatagaaggatattgcgttttttaaaattaaaatagtagcagttattaatttaaaatattctctttaaaaaatggaggcacaatgaaagtatcattagttataccagtttataatggagcaaatcatatcaaagaaacattagaaagtattatagcgcagacctaccccaatttggaaatattaatcattgatgatgggtcaactgataatacatcaactatatgtcagaagtatgtttctaaagataagcgcatgaaatatatatataaaaaaaatggaggtcaatcttctgctagaaatttaggcgttaaactatctacaagtaattggatagctttcttagattctgatgatctttgttcgccattttatatagaatgtctagtgagaattcaacatgaaactggtgctgaccttgtaataactggaattgaggatcacccttgttacagaacggcaaaactttcatttgataaactatcttactcagtactcagtgtggaagagacactaatagaaatgtattatgataactatggtatttctccattagggaagttatattccaaagggttattattaaaatatccataccctattggtaaaattcatgaagatacagacacaacttataaactgattgcaagtagtacacgaattgcattgagcaaagagaaaacagtatatgtcaaagggcaacctaatagtactaccagacagtcatacaaagagaatatgttatattattttgaagcaatgcaacataatagagattttatccttgaacattattcagataaagaactaattgatgctttaaattataaaactgtaattggaggaatgtggatcgttaataagcttttagaaggagataagtacgattatattaattctttgagaagtgaatatagaaaatattataagagtatattatttaataagcgagttacaattaaagaaaaaataaaatatttttcatttatttcgtcaatgaatttatttttatttttaaaaaaattttataactataatgggagataaattatgaagatgattagtatcattatcccagatctgtaaggaaggaaaatgatacctaaagttatacattattgttggtttggaggaaatcccttaccagataatttaaagaaatatataaaaacttggagagaacaatgtccggattatgaaattattgaatggaatgagcataattatgatgttagtaaaaatgtttttatgagagaagcatatactaagaagaattttgcttatgtttctgactatgcaagattggatattatttatacttatggggggttctatctagatactgatgtggagcttttaaaaagtttagatcctttgaggattcatgagtgttttctagcaagggagattagttgtgatgtgaatacaggattaataattggcgctgttaaaggacatcactttttaaaatcaaatatgtctatatatgacaaaagtgatttaacttctcttaataagacatgtgtagaggttacaactaatttattgataaacagagggcttaagaataagaatattattcaaaagattgatgatataacaatatatccgagaaattattttaatccaaagaatttattaacaggtaaggttgattgtctgactagtgttacctattctatacatcattacgaaggaagttggaaaagttcttcatttatttcagattctctaaagattagagtaaggctcataattgattttttatttggatatggtacttatagaatgcttctaaggtttctaaagttaaagaaatagttattattgtaatagaaaggacgaaatgttatatatttttactcctacatttaacagaggctaccgattatcctatctgtatgattctctatgtaatcagacaaataaaaattttatatggttgattgttgatgatggttctgaggacagtactaaggaaattgtaagtaattatataaaagagaataaagttagtattgtatatctgtataaacgtaatggcggtaagcattcagcctataatctagcaatgagatatatgcaaccaagtgattatcatgtatgtgtagatagtgatgattggttattagaagatgcagttgaaattatttttaaagatttagagagcctaacgttgtctaatagatatgtaggccttgtttatcctcgatattcattaaaccaaggtaataattggttgaatcccaaaatattagaagttaatattcctgatttaaaatataaatatcacttaaaaattgaaacttgtattgttattaataatgcttatttagtagatttcgaatttccttgtttcgaaggggagaatttcttatcagaagaaataatgtatatatatttatcaaaaaagggatacttttgtccccaaaataggaaaatctattgttttgactacttagaagatggtttgacttctaatatttttaaattatggagaaaaaatttcaaagggactattttttcattagaaaattcatatatgtatgtaatgtcttttcctaatatatttgatcgctggtggtcagctataaagataaaaatgaacatacaggctttgaaaatgactactttaggggtaatacctacactaaagagtgaagaggctgggtggaaaattttgttgggttttagctatctttggaaggtagcaaggtttaaaaagagcgagtaaagaatggaattaatttcaataatagtgcctgtatataatggagaaatatatattgggagatgtctagatagtattcttgagcaaacttatcagaatttagagattatcataattgatgatggctctagtgatcgcacaggggatatttgcgaaaagtatttattggaagacaggcgaataaaatatttctatcaagaaaacagaggccaatcagttgcacgtaacaacggagtacttaggtgtacaggagattggattgcttttttagacagtgatgatgtttatcttccttattcaattgaggttatgtacaacatacagaaagctaccaatgccgatattgttttaactagtattggtaacttcaacaatacttacaatactagtattaattcacagtatttaaaagagattaaactttatactttagaggtggcgttggaagaaatgtattatgggaaaacttatggagtttccccgttagcaaaattatatccgagaagtaatttattgagtaatccatatccagaaggaaaaattcatgaagatatggatacaacttttaagcttatatcttgcgcttcaaaaatagcagtttgtgatattgtcactgcagtggtgtattttagcgataatagtactactaggacaaaattcaatgagagaatgttgtatttttttgaggcaattcaaaataatattgtatttataaatctaaattttccacataatacatctttaatctctgctgtaatatataatgaagtatttggtggaatagatatatgtgggaagatgattgattttaaattatatgatacagttgattattacagaaaaaaatatagaaaatattttaagactattctctttaataataggattagtgtgaaggagaaggttaaatatatattatttatttcctctataagatatttcactattgtaagaaaaatatataatttgaggttatcaaggaatggataaagttagtatcattatcccagatct